(19) United States
(12) Patent Application Publication
(10) Pub. No.: US 2024/0002544 A1
(43) Pub. Date: Jan. 4, 2024

(54) CD28 BISPECIFIC ANTIBODIES FOR TARGETED T CELL ACTIVATION

(71) Applicant: Novimmune SA, Geneva (CH)

(72) Inventors: Sara MAJOCCHI, Grand Lancy (CH); Krzysztof MASTERNAK, Mollens (CH); Limin SHANG, Bellevue (CH); Nicolas FISCHER, Lancy (CH); Xavier CHAUCHET, Thusy (FR)

(21) Appl. No.: 18/180,116

(22) Filed: Mar. 7, 2023

Related U.S. Application Data
(60) Provisional application No. 63/323,893, filed on Mar. 25, 2022.
(60) Provisional application No. 63/317,491, filed on Mar. 7, 2022.

(51) Int. Cl. C07K 16/46; A61P 35/00

(52) U.S. Cl. CPC C07K 16/468; C07K 2317/31; C07K 2317/565; A61P 35/00

(57) ABSTRACT

The present invention relates to antibody arms which bind agonistically to human CD28. The present invention relates to bispecific antibodies which bind to human carcinoembryonic antigen (CEA) and human CD28 using the agonist anti-CD28 arms described herein. The present invention relates to bispecific antibodies which bind to human mesothelin (MSLN) and human CD28 using the agonist anti-CD28 arms described herein.

### RELATED APPLICATIONS

The application claims priority to, and the benefit of, U.S. Provisional Application No. 63/317,491, filed on Mar. 7, 2022, and U.S. Provisional Application No. 63/323,893, filed on Mar. 25, 2022, the contents of each of which are incorporated by reference herein in their entireties.

### INCORPORATION BY REFERENCE OF SEQUENCE LISTING

The contents of the electronic sequence listing (NOVI_050_001US_SeqList_ST26.xml; Size: 129,730 bytes; and Date of Creation: Mar. 6, 2023) are herein incorporated by reference in its entirety.

### FIELD

The present invention relates generally agonist fully human and κλ-body compatible CD28 binding domains of defined affinity usefully in the generation of bispecific antibodies (bsAb). Specifically, the CD28 binding domains are paired with anti-tumor-associated antigen (TAA) binding domains for the generation of TAA×CD28 bsAb, in particular CEA×CD28 and MSLN×CD28 bispecific antibodies.

### BACKGROUND OF THE INVENTION

In the past years, novel approaches to stimulate the body's own immune cells to better attack and kill cancer cells have been developed. Examples of successful cancer immunotherapies are monoclonal antibodies capable of blocking so-called immune checkpoints. Currently approved immune checkpoint inhibitors (ICI) block CTLA-4 (e.g., Ipilimumab, sold under the brand name Yervoy), PD-1 (e.g., Pembrolizumab, sold under the brand name Keytruda and Cemiplimab, sold under the brand name Libtayo) and PD-L1 (e.g., Atezolizumab, sold under the brand name Tecentriq). Durable anti-tumor responses can be obtained in a range of cancer types using ICIs. Unfortunately, responses are limited to a patient subset, and many cancer types are known to be intrinsically resistant to ICI monotherapies.

Other approved cancer immunotherapies include T cell bispecific antibodies—bridging T cells to target cells expressing a tumor associated antigen (TAA), via the CD3 receptor on T cells and on Chimeric Antigen Receptor (CAR) T cells. Despite the very good anti-tumor responses observed with treatments using T cell bispecific antibodies or CAR T cells in hematological malignancies, there have been no significant breakthroughs of these approaches in the context of solid cancers to date, leaving many cancer patients with no therapeutic options.

Therefore, and in spite of the success of these immunotherapies in some cancer types, a large fraction of cancer patients lacks valid therapeutic options, highlighting the need for new treatments. The use of molecules capable of activating the immune system by targeting costimulatory signals on T cells has not been fully explored and may open the way to novel therapeutic options for solid cancer patients.

A need exists for compositions and methods for targeting T-cell activation useful for treating solid cancers. Provided herein are methods and compositions addressing this need.

### SUMMARY OF THE INVENTION

This disclosure provides a bispecific antibody comprising: a. a first antigen binding domain that binds to CD28; wherein the first antigen binding domain comprises: i. a first heavy chain variable region having a complementarity determining region 1 (CDRH1) comprising the amino acid sequence of (SEQ ID NO: 1); a complementarity determining region 2 (CDRH2) comprising the amino acid sequence of (SEQ ID NO: 2); and a complementarity determining region 3 (CDRH3) comprising the amino acid sequence of (SEQ ID NO: 3); and ii. a first light chain variable region having: 1. a CDRL1 comprising the amino acid sequence of SEQ ID NO: 23; a CDRL2 comprising the amino acid sequence of SEQ ID NO: 24; and a CDRL3 comprising the amino acid sequence of SEQ ID NO: 25; 2. a CDRL1 comprising the amino acid sequence of SEQ ID NO: 26; a CDRL2 comprising the amino acid sequence of SEQ ID NO: 27; and a CDRL3 comprising the amino acid sequence of SEQ ID NO: 28; 3. a CDRL1 comprising the amino acid sequence of SEQ ID NO: 29; a CDRL2 comprising the amino acid sequence of SEQ ID NO: 30; and a CDRL3 comprising the amino acid sequence of SEQ ID NO: 31; 4. a CDRL1 comprising the amino acid sequence of SEQ ID NO: 32; a CDRL2 comprising the amino acid sequence of SEQ ID NO: 33; and a CDRL3 comprising the amino acid sequence of SEQ ID NO: 34; 5. a CDRL1 comprising the amino acid sequence of SEQ ID NO: 35;
a CDRL2 comprising the amino acid sequence of SEQ ID NO: 36; and a CDRL3 comprising the amino acid sequence of SEQ ID NO: 37; 6. a CDRL1 comprising the amino acid sequence of SEQ ID NO: 38; a CDRL2 comprising the amino acid sequence of SEQ ID NO: 39; and a CDRL3 comprising the amino acid sequence of SEQ ID NO: 40; 7. a CDRL1 comprising the amino acid sequence of SEQ ID NO: 41; a CDRL2 comprising the amino acid sequence of SEQ ID NO: 42; and a CDRL3 comprising the amino acid sequence of SEQ ID NO: 43; 8. a CDRL1 comprising the amino acid sequence of SEQ ID NO: 44; a CDRL2 comprising the amino acid sequence of SEQ ID NO: 45; and a CDRL3 comprising the amino acid sequence of SEQ ID NO: 46; 9. a CDRL1 comprising the amino acid sequence of SEQ ID NO: 47; a CDRL2 comprising the amino acid sequence of SEQ ID NO: 48; and a CDRL3 comprising the amino acid sequence of SEQ ID NO: 49; 10. a CDRL1 comprising the amino acid sequence of SEQ ID NO: 50; a CDRL2 comprising the amino acid sequence of SEQ ID NO: 51; and a CDRL3 comprising the amino acid sequence of SEQ ID NO: 52; 11. a CDRL1 comprising the amino acid sequence of SEQ ID NO: 53; a CDRL2 comprising the amino acid sequence of SEQ ID NO: 54; and a CDRL3 comprising the amino acid sequence of SEQ ID NO: 55; 12. a CDRL1 comprising the amino acid sequence of SEQ ID NO: 56; a CDRL2 comprising the amino acid sequence of SEQ ID NO: 57; and a CDRL3 comprising the amino acid sequence of SEQ ID NO: 58; 13. a CDRL1 comprising the amino acid sequence of SEQ ID NO: 59; a CDRL2 comprising the amino acid sequence of SEQ ID NO: 60; and a CDRL3 comprising the amino acid sequence of SEQ ID NO: 61; 14. a CDRL1 comprising the amino acid sequence of SEQ ID NO: 62; a CDRL2 comprising the amino acid sequence of SEQ ID NO: 63; and a CDRL3 comprising the amino acid sequence of SEQ ID NO: 64; 15. a CDRL1 comprising the amino acid sequence of SEQ ID NO: 65; a CDRL2 comprising the amino acid sequence of SEQ ID NO: 66; and a CDRL3 comprising the amino acid sequence of SEQ ID NO: 67; or 16. a CDRL1 comprising the amino acid sequence of SEQ ID NO: 68; a CDRL2 comprising the amino acid sequence of SEQ ID NO: 69; and a CDRL3 comprising the amino acid sequence of SEQ ID NO: 70; 17. a CDRL1 comprising the amino acid sequence of SEQ ID NO: 71; a CDRL2 comprising the amino acid sequence of SEQ ID NO: 72; and a CDRL3 comprising the amino acid sequence of SEQ ID NO: 73; 18. a CDRL1 comprising the amino acid sequence of SEQ ID NO: 74; a CDRL2 comprising the amino acid sequence of SEQ ID NO: 75; and a CDRL3 comprising the amino acid sequence of SEQ ID NO: 76; or 19. a CDRL1 comprising the amino acid sequence of SEQ ID NO: 77; a CDRL2 comprising the amino acid sequence of SEQ ID NO: 78; and a CDRL3 comprising the amino acid sequence of SEQ ID NO: 79; and b. a second antigen binding domain that bind a tumor associated antigen (TAA), wherein the second antigen binding domain comprises: i. a second heavy chain variable region having a CDRH1 comprising the amino acid sequence of SEQ ID NO: 1; a CDRH2 comprising the amino acid sequence of SEQ ID NO: 2; and a CDRH3 comprising the amino acid sequence of SEQ ID NO: 3.

In some embodiments, the TAA is CEA. In some aspects, the TAA is mesothelin (MSLN).

In some embodiments, the first light chain variable region of: a. part a. ii. 1. comprises the amino acid sequence of SEQ ID NO: 80; b. part a. ii. 2. comprises the amino acid sequence of SEQ ID NO: 81; c. part a. ii. 3. comprises the amino acid sequence of SEQ ID NO: 82; d. part a. ii. 4. comprises the amino acid sequence of SEQ ID NO: 83; e. part a. ii. 5. comprises the amino acid sequence of SEQ ID NO: 84; f. part a. ii. 6. comprises the amino acid sequence of SEQ ID NO: 85; g. part a. ii. 7. comprises the amino acid sequence of SEQ ID NO: 86; h. part a. ii. 8. comprises the amino acid sequence of SEQ ID NO: 87; i. part a. ii. 9. comprises the amino acid sequence of SEQ ID NO: 88; j. part a. ii. 10. comprises the amino acid sequence of SEQ ID NO: 89; k. part a. ii. 11. comprises the amino acid sequence of SEQ ID NO: 90; l. part a. ii. 12. comprises the amino acid sequence of SEQ ID NO: 91; m. part a. ii. 13. comprises the amino acid sequence of SEQ ID NO: 92; n. part a. ii. 14. comprises the amino acid sequence of SEQ ID NO: 93; o. part a. ii. 15. comprises the amino acid sequence of SEQ ID NO: 94; p. part a. ii. 16. comprises the amino acid sequence of SEQ ID NO: 95; q. part a. ii. 17. comprises the amino acid sequence of SEQ ID NO: 96; r. part a. ii. 18. comprises the amino acid sequence of SEQ ID NO: 97; or s. part a. ii. 19. comprises the amino acid sequence of SEQ ID NO: 98.

In some embodiments, the first light chain of: a. part a. ii. 1. comprises the amino acid sequence of SEQ ID NO: 99; b. part a. ii. 2. comprises the amino acid sequence of SEQ ID NO: 100; c. part a. ii. 3. comprises the amino acid sequence of SEQ ID NO: 101; d. part a. ii. 4. comprises the amino acid sequence of SEQ ID NO: 102; e. part a. ii. 5. comprises the amino acid sequence of SEQ ID NO: 103; f. part a. ii. 6. comprises the amino acid sequence of SEQ ID NO: 104; g. part a. ii. 7. comprises the amino acid sequence of SEQ ID NO: 105; h. part a. ii. 8. comprises the amino acid sequence of SEQ ID NO: 106; i. part a. ii. 9. comprises the amino acid sequence of SEQ ID NO: 107; j. part a. ii. 10. comprises the amino acid sequence of SEQ ID NO: 108; k. part a. ii. 11. comprises the amino acid sequence of SEQ ID NO: 109; l. part a. ii. 12. comprises the amino acid sequence of SEQ ID NO: 110; m. part a. ii. 13. comprises the amino acid sequence of SEQ ID NO: 111; n. part a. ii. 14. comprises the amino acid sequence of SEQ ID NO: 112; o. part a. ii. 15. comprises the amino acid sequence of SEQ ID NO: 113; p. part a. ii. 16. comprises the amino acid sequence of SEQ ID NO: 114; q. part a. ii. 17. comprises the amino acid sequence of SEQ ID NO: 115; r. part a. ii. 18. comprises the amino acid sequence of SEQ ID NO: 116; or s. part a. ii. 19. comprises the amino acid sequence of SEQ ID NO: 117.

In some embodiments, the second antigen binding domain comprises: ii. a second light chain variable region having: 1. a CDR1 comprising the amino acid sequence of SEQ ID NO: 8; a CDR2 comprising the amino acid sequence of SEQ ID NO: 9; and a CDR3 comprising the amino acid sequence of SEQ ID NO: 10; 2. a CDR1 comprising the amino acid sequence of SEQ ID NO: 11; a CDR2 comprising the amino acid sequence of SEQ ID NO: 12; and a CDR3 comprising the amino acid sequence of SEQ ID NO: 13; or 3. a CDR1 comprising the amino acid sequence of SEQ ID NO: 14; a CDR2 comprising the amino acid sequence of SEQ ID NO: 15 and a CDR3 comprising the amino acid sequence of SEQ ID NO: 16.

In some embodiments, the second light chain variable region of: a. part b. ii. 1. comprises the amino acid sequence of SEQ ID NO: 17; b. part b. ii. 2. comprises the amino acid sequence of SEQ ID NO: 18; or c. part b. ii. 3. comprises the amino acid sequence of SEQ ID NO: 19.

In some embodiments, the second light chain of: a. part b. ii. 1. comprises the amino acid sequence of SEQ ID NO: 20; b. part b. ii. 2. comprises the amino acid sequence of SEQ ID NO: 21; or c. part b. ii. 3. comprises the amino acid sequence of SEQ ID NO: 22.

In some embodiments, the second antigen binding domain comprises: ii. a second light chain variable region having: 1. a CDR1 comprising the amino acid sequence of SEQ ID NO: 128; a CDR2 comprising the amino acid sequence of SEQ ID NO: 129; and a CDR3 comprising the amino acid sequence of SEQ ID NO: 130; 2. a CDR1 comprising the amino acid sequence of SEQ ID NO: 131; a CDR2 comprising the amino acid sequence of SEQ ID NO: 132; and a CDR3 comprising the amino acid sequence of SEQ ID NO: 133; 3. a CDR1 comprising the amino acid sequence of SEQ ID NO: 134; a CDR2 comprising the amino acid sequence of SEQ ID NO: 135; and a CDR3 comprising the amino acid sequence of SEQ ID NO: 136; 4. a CDR1 comprising the amino acid sequence of SEQ ID NO: 137; a CDR2 comprising the amino acid sequence of SEQ ID NO: 138; and a CDR3 comprising the amino acid sequence of SEQ ID NO: 139; or 5. a CDR1 comprising the amino acid sequence of SEQ ID NO: 140; a CDR2 comprising the amino acid sequence of SEQ ID NO: 141; and a CDR3 comprising the amino acid sequence of SEQ ID NO: 142.

In some embodiments, the second light chain variable region of: a. part b. ii. 1. comprises the amino acid sequence of SEQ ID NO: 143; b. part b. ii. 2. comprises the amino acid sequence of SEQ ID NO: 144; c. part b. ii. 3. comprises the amino acid sequence of SEQ ID NO: 145. d. part b. ii. 4. comprises the amino acid sequence of SEQ ID NO: 146; or e. part b. ii. 5. comprises the amino acid sequence of SEQ ID NO: 147.

In some embodiments, the second light chain of: a. part b. ii. 1. comprises the amino acid sequence of SEQ ID NO: 148; b. part b. ii. 2. comprises the amino acid sequence of SEQ ID NO: 149; c. part b. ii. 3. comprises the amino acid sequence of SEQ ID NO: 150; d. part b. ii. 4. comprises the amino acid sequence of SEQ ID NO: 151; or e. part b. ii. 5. comprises the amino acid sequence of SEQ ID NO: 152.

In some embodiments, the first heavy chain variable region and the second heavy chain variable region comprises the amino acid sequence of SEQ ID NO: 4. In some embodiments, the first heavy chain and the second heavy chain comprises the amino acid sequence of SEQ ID NO: 6 or SEQ ID NO: 7.

In some embodiments, the first light chain is a kappa and the second light chain is a lambda. In some embodiments, the first light chain is a lambda and the second light chain is a kappa.

In some embodiments, the bispecific antibody comprises an Fc domain comprising one or more amino acid substitutions that reduce binding to an activating Fc receptor and/or reduce effector function. In some embodiments, the amino acid substitution comprises a L234A and L235A substitution. In some embodiments, the amino acid substitution comprises a P329A, P329G or P329R substitution. In some embodiments, the antibody has an IgG isotype. In some embodiments, the antibodies are human.

The disclosure provides a composition comprising any one of the bispecific antibodies of the disclosure. In some embodiments, the composition comprising the bispecific antibody enables tumor-specific T cell activation.

The disclosure provides a method of reducing the proliferation of and/or killing a tumor cell comprising contacting the cell with any one of the compositions of the disclosure.

The disclosure provides a method of treating a cancer in a subject comprising administering to the subject any one of the compositions of the disclosure.

The disclosure provides an antibody comprising an antigen binding domain that binds to CD28; wherein the antigen binding domain comprises: 1. a CDRL1 comprising the amino acid sequence of SEQ ID NO: 23; a CDRL2 comprising the amino acid sequence of SEQ ID NO: 24; and a CDRL3 comprising the amino acid sequence of SEQ ID NO: 25; 2. a CDRL1 comprising the amino acid sequence of SEQ ID NO: 26; a CDRL2 comprising the amino acid sequence of SEQ ID NO: 27; and a CDRL3 comprising the amino acid sequence of SEQ ID NO: 28; 3. a CDRL1 comprising the amino acid sequence of SEQ ID NO: 29; a CDRL2 comprising the amino acid sequence of SEQ ID NO: 30; and a CDRL3 comprising the amino acid sequence of SEQ ID NO: 31; 4. a CDRL1 comprising the amino acid sequence of SEQ ID NO: 32; a CDRL2 comprising the amino acid sequence of SEQ ID NO: 33; and a CDRL3 comprising the amino acid sequence of SEQ ID NO: 34; 5. a CDRL1 comprising the amino acid sequence of SEQ ID NO: 35;
a CDRL2 comprising the amino acid sequence of SEQ ID NO: 36; and a CDRL3 comprising the amino acid sequence of SEQ ID NO: 37; 6. a CDRL1 comprising the amino acid sequence of SEQ ID NO: 38; a CDRL2 comprising the amino acid sequence of SEQ ID NO: 39; and a CDRL3 comprising the amino acid sequence of SEQ ID NO: 40; 7. a CDRL1 comprising the amino acid sequence of SEQ ID NO: 41; a CDRL2 comprising the amino acid sequence of SEQ ID NO: 42; and a CDRL3 comprising the amino acid sequence of SEQ ID NO: 43; 8. a CDRL1 comprising the amino acid sequence of SEQ ID NO: 44; a CDRL2 comprising the amino acid sequence of SEQ ID NO: 45; and a CDRL3 comprising the amino acid sequence of SEQ ID NO: 46; 9. a CDRL1 comprising the amino acid sequence of SEQ ID NO: 47; a CDRL2 comprising the amino acid sequence of SEQ ID NO: 48; and a CDRL3 comprising the amino acid sequence of SEQ ID NO: 49; 10. a CDRL1 comprising the amino acid sequence of SEQ ID NO: 50;
a CDRL2 comprising the amino acid sequence of SEQ ID NO: 51; and a CDRL3 comprising the amino acid sequence of SEQ ID NO: 52; 11. a CDRL1 comprising the amino acid sequence of SEQ ID NO: 53; a CDRL2 comprising the amino acid sequence of SEQ ID NO: 54; and a CDRL3 comprising the amino acid sequence of SEQ ID NO: 55; 12. a CDRL1 comprising the amino acid sequence of SEQ ID NO: 56; a CDRL2 comprising the amino acid sequence of SEQ ID NO: 57; and a CDRL3 comprising the amino acid sequence of SEQ ID NO: 58; 13. a CDRL1 comprising the amino acid sequence of SEQ ID NO: 59; a CDRL2 comprising the amino acid sequence of SEQ ID NO: 60; and a CDRL3 comprising the amino acid sequence of SEQ ID NO: 61; 14. a CDRL1 comprising the amino acid sequence of SEQ ID NO: 62; a CDRL2 comprising the amino acid sequence of SEQ ID NO: 63; and a CDRL3 comprising the amino acid sequence of SEQ ID NO: 64; 15. a CDRL1 comprising the amino acid sequence of SEQ ID NO: 65; a CDRL2 comprising the amino acid sequence of SEQ ID NO: 66; and a CDRL3 comprising the amino acid sequence of SEQ ID NO: 67; or 16. a CDRL1 comprising the amino acid sequence of SEQ ID NO: 68; a CDRL2 comprising the amino acid sequence of SEQ ID NO: 69; and
a CDRL3 comprising the amino acid sequence of SEQ ID NO: 70; 17. a CDRL1 comprising the amino acid sequence of SEQ ID NO: 71; a CDRL2 comprising the amino acid sequence of SEQ ID NO: 72; and a CDRL3 comprising the amino acid sequence of SEQ ID NO: 73; 18. a CDRL1 comprising the amino acid sequence of SEQ ID NO: 74; a CDRL2 comprising the amino acid sequence of SEQ ID NO: 75; and a CDRL3 comprising the amino acid sequence of SEQ ID NO: 76; or 19. a CDRL1 comprising the amino acid sequence of SEQ ID NO: 77; a CDRL2 comprising the amino acid sequence of SEQ ID NO: 78; and a CDRL3 comprising the amino acid sequence of SEQ ID NO: 79.

In some embodiments, the antibody is a F(ab) fragment, a F(ab′)2 fragment, and Fv fragment or a single chain Fv fragment. In some embodiments, the antibody is monospecific. In some embodiments, the antibody is monovalent.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

Other features and advantages of the invention will be apparent from and encompassed by the following detailed description and claims.

### BRIEF DESCRIPTION OF THE DRAWINGS

FIG. 1A, FIG. 1B, FIG. 1C are a series of graphs showing concentration dependent binding of the anti-CD28 arms of the invention to CD28 expressing Jurkat cells. The anti-CD28 arms were tested as bivalent, monoclonal antibodies (hIgG1). hIgG1: isotype control; TGN1412 (hIgG4): positive control. FIGS. 1A, 1B and 1C show candidates identified during consecutive phage-display selection campaigns.

FIG. 2A, FIG. 2B, FIG. 2C, FIG. 2D are a series of graphs showing concentration dependent binding of the CEA×CD28 bispecific antibodies of the invention to CD28 expressing Jurkat cells (FIG. 2A and FIG. 2B), CEA expressing LS174T cells (FIG. 2C) and CD28 and CEA double negative TIB153 cells (FIG. 2D). A panel of CEA×CD28 bsAbs was generated using the high-affinity anti-CEA arm AC84, an IgG1 Fc with the three mutations LALAPA, and various anti-CD28 arms of different affinities. hIgG1: isotype control; TGN1412: positive control; 1a28/AC84/N, CEA_CD28_V8 and CEA_CD28_V15: CEA×CD28 reference bispecific antibodies.

FIG. 3A, FIG. 3B, FIG. 3C, FIG. 3D are a series of graphs showing proliferation of human CD4+ and CD8+ T cells (FIGS. 3A and 3C, and 3B and 3D, respectively) from healthy donor PBMCs mediated by the CEA×CD28 bispecific antibodies of the invention, following the so-called wet-coating (FIGS. 3A and 3C) or dry-coating (FIGS. 3B and 3D) of the antibodies on test plates. A panel of anti-CD28 arms was tested for superagonist activity in the bispecific format. Except for “AI19”, none of the anti-CD28 arms of the invention displayed superagonistic activity in this kind of assay. PBS: vehicle control; TGN1412: positive control; 1a28/AC84/N, CEA_CD28_V8 and CEA_CD28_V15: CEA×C28 reference bispecific antibodies; mAb 14226P2, mAb V8 and mAb V15: reference monoclonal antibodies.

FIG. 4 is a series of graphs showing concentration dependent IL-2 reporter cell activation of the CEA×CD28 bispecific antibodies of the invention as measured by luminescence readout in presence of CEA expressing MKN-45 cells and of a fixed concentration (1 nM) of CEA×CD3. Different levels of CD28 co-stimulation induced by the CEA×CD28 bsAbs of the invention are observed, reflecting the distinct binding capacity of these anti-CD28 arms. DR=Dose Response; w/o target: CD3 and CD28 bsAbs are incubated in presence of effector/reporter cells, but in the absence of the TAA-positive target cells.

FIG. 5A, FIG. 5B, FIG. 5C, FIG. 5D are a series of graphs showing T-cell retargeted killing/lysis of LS174T cells by the CEA×CD28 bispecific antibodies of the invention when combined with a CEA×CD3 bsAb. Effector cells are PBMCs derived from two different healthy donors (FIGS. 5A and 5B). CEA×CD28 bsAbs synergize with CEA×CD3 to kill CEA-positive target cells. No killing is induced in the absence of signal 1 (FIGS. 5C and 5D, data obtained with the same PBMC donors used in FIGS. 5A and 5B, respectively). Y4L3-1/N: untargeted CD3 monovalent antibody; 1a28/AC84/N: CEA×CD28 reference bispecific antibody. CD28 bsAb concentration used: 1 μg/mL.

FIG. 6A, FIG. 6B are a series of graphs showing T-cell retargeted killing/lysis of KATO-III (FIG. 6A) and HT-29 (FIG. 6B) cells by the CEA×CD28 bispecific antibodies of the invention when combined with a CEA×CD3 bsAb. Synergy between CEA×CD3 and CEA×CD28 bsAb in killing CEA-positive target cells is observed also with these lower expressing cell lines. 1a28/AC84/N: CEA×CD28 reference bispecific antibody. CD28 bsAb concentration used: 2.5 g/mL.

FIG. 7A, FIG. 7B are a series of graphs showing upregulation of the T cell activation marker CD25 on human CD4+ and CD8+ T cells (FIGS. 7A and 7B, respectively). PBMCs were co-cultured with CEA-positive LS174T cells in presence of either CEA×CD3 alone (to trigger T cell activation signal 1) or in combination to the CEA×CD28 bispecific antibodies of the invention (providing T cell activation signal 2). The combination of CEA×CD3 and CEA×CD28 bsAbs induces a much stronger activation of both CD4+ and CD8+ T cells than the CEA×CD3 monotherapy. 1a28/AC84/N: CEA×CD28 reference bispecific antibody.

FIG. 8A, FIG. 8B are a series of graphs showing proliferation of human CD4+ and CD8+ T cells (FIGS. 8A and 8B, respectively). PBMCs were co-cultured with CEA-positive LS174T cells in presence of either CEA×CD3 alone (to trigger T cell activation signal 1) or in combination to the CEA×CD28 bispecific antibodies of the invention (providing T cell activation signal 2). The combination of CEA×CD3 and CEA×CD28 bsAbs strongly increases the percentage of proliferative CD4+ and CD8+ T cells compared to CEA×CD3 monotherapy. 1a28/AC84/N: CEA×CD28 reference bispecific antibody.

FIG. 9A, FIG. 9B, FIG. 9C, FIG. 9D, FIG. 9E, FIG. 9F are a series of graphs showing secretion of Granzyme B (FIG. 9A); IFN-7 (FIG. 9B); TNF-α (FIG. 9C); IL-2 (FIG. 9D); IL-6 (FIG. 9E); IL-10 (FIG. 9F) mediated by the CEA×CD28 bispecific antibodies of the invention when combined with a CEA×CD3 bsAb following the co-culture of PBMC with CEA-positive LS174T tumor cells. Induction of cytokines by CEA×CD3 is increased when T cells are simultaneously co-stimulated by CEA×CD28 bsAbs. 1a28/AC84/N: CEA×CD28 reference bispecific antibody.

FIG. 10 is a schematic diagram of the experimental design of the in vivo efficacy study shown in FIG. 11, where CEA×CD28 bispecific antibodies AI3AC84/N and AI5AC84/N were tested in combination with CEA×CD3 in a LS174T:PBMCs co-grafting model in NOG mice.

FIG. 11A, FIG. 11B are a series of graphs showing in vivo anti-tumor efficacy of the CEA×CD28 bispecific antibodies AI3AC84/N and AI5AC84/N in the LS174T:PBMCs co-grafting model described on FIG. 10. The average tumor volume (FIG. 11A) and the growth of tumors in individual mice (FIG. 11B) are shown for all treatment groups. In vivo anti-tumoral activity of CEA×CD3 is enhanced by both CEA×CD28 bsAbs.

FIG. 12 is a schematic diagram of the experimental design of the in vivo efficacy study shown on FIG. 13, where CEA×CD28 bispecific antibodies AI3AC84/N (in a dose range) and AI10AC84/N were tested in combination with CEA×CD3 in a LS174T:PBMCs co-grafting model in NOG mice.

FIG. 13A, FIG. 13B are a series of graphs showing in vivo anti-tumor efficacy of the CEA×CD28 bispecific antibodies AI3AC84/N (in a dose range) and AI10AC84/N in the LS174T:PBMCs co-grafting model described on FIG. 12. The average tumor volume (FIG. 13A) and the growth of tumors in individual mice (FIG. 13B) are shown for all treatment groups. In vivo anti-tumoral activity of CEA×CD3 is enhanced by both CEA×CD28 bsAbs. However, at equivalent treatment dosing, antibody AI3AC84/N led to better tumor control than AI10AC84/N.

FIG. 14A, FIG. 14B, FIG. 14C, FIG. 14D are a series of graphs showing concentration dependent binding of the MSLN×CD28 bispecific antibodies of the invention to MSLN expressing H226 (FIG. 14A) and OVCAR-3 (FIG. 14B) cells, to CD28 expressing Jurkat cells (FIG. 14C), and to CD28 and MSLN double negative TIB153 cells (FIG. 14D). A panel of huIgG1-LALAPA MSLN×CD28 bsAbs was generated using the high-affinity anti-MSLN arms O30, O35 and O41 coupled to either “AI5” or “AI10” anti-CD28 arms. hIgG1: isotype control.

FIG. 15A, FIG. 15B, FIG. 15C, FIG. 15D, FIG. 15E, FIG. 15F are a series of graphs showing effects of MSLN×CD3 and MSLN×CD28 bispecific antibody combinations on target cell killing (FIGS. 15A and 15D), T cell activation (FIGS. 15B and 15E) and T cell proliferation (FIGS. 15C and 15F). MSLN×CD28 bsAbs synergize with a HPN536 analog to kill MSLN-positive target cells and to induce T cell activation and proliferation. No killing, T cell activation or T cell proliferation is induced when HPN536 analog is replaced by Y4L3-1/N, an untargeted CD3 monovalent antibody unable to provide T cell activation signal 1.

FIG. 16A, FIG. 16B, FIG. 16C, FIG. 16D, FIG. 16E, FIG. 16F are a series of graphs showing secretion of Granzyme B (FIG. 16A); IFN-7 (FIG. 16B); TNF-α (FIG. 16C); IL-2 (FIG. 16D); IL-6 (FIG. 16E); IL-10 (FIG. 16F) mediated by the MSLN×CD28 bispecific antibodies of the invention when combined with a HPN536 analog following the co-culture of PBMC with MSLN-positive H226 tumor cells. Induction of cytokines by HPN536 analog is importantly increased when T cells are simultaneously co-stimulated by MSLN×CD28 bsAbs.

FIG. 17A, FIG. 17B, FIG. 17C, FIG. 17D are a series of graphs showing concentration dependent binding of exemplary MSLN×CD28 bispecific antibodies of the invention to MSLN expressing H226 (FIG. 17A) and OVCAR-3 (FIG. 17B) cells, to CD28 expressing Jurkat cells (FIG. 17C), and to CD28 and MSLN double negative TIB153 cells (FIG. 17D). Exemplary MSLN×CD28 bsAbs generated using the high-affinity anti-MSLN arms O35 and O41 coupled to AI3 anti-CD28 arms are shown. hIgG1: isotype control.

FIG. 18A, FIG. 18B, FIG. 18C, FIG. 18D are a series of graphs showing T cell retargeted killing/lysis of OVCAR3 cells by exemplary MSLN×CD28 bispecific antibodies of the invention when combined with HPN536 analog, a MSLN×CD3 bsAb, in presence of different E:T ratios (10:1 (FIG. 18A), 3:1 (FIG. 18B), 1:1 (FIG. 18C) and 1:3 (FIG. 18D)). MSLN×CD28 bsAbs synergize with MSLN×CD3 to kill MSLN-positive target cells. Synergy is particularly visible at unfavorable E:T ratios, where the combination compensates for the loss of efficacy of the MSLN×CD3 bsAb alone. No killing is induced in the absence of signal 1 (CD28 bsAbs alone). CD28 bsAb concentration used: 2.5 μg/mL.

FIG. 19A, FIG. 19B, FIG. 19C, FIG. 19D are a series of graphs showing T cell retargeted killing/lysis of CEA/MSLN double positive HPAC cells by exemplary CD28 bispecific antibodies of the invention when combined with CEA×CD3, in presence of different E:T ratios (10:1 (FIG. 19A), 3:1 (FIG. 19B), 1:1 (FIG. 19C) and 1:3 (FIG. 19D)). Both CEA×CD28 and MSLN×CD28 bsAbs synergize with CEA×CD3 to kill target cells. Synergy is particularly visible at unfavorable E:T ratios, where the combination compensates for the loss of efficacy of the CEA×CD3 bsAb alone. No killing is induced in the absence of signal 1 (CD28 bsAbs alone). CD28 bsAb concentration used: 2.5 μg/mL.

FIG. 20A, FIG. 20B, FIG. 20C, FIG. 20D are a series of graphs showing upregulation of the T cell activation marker CD25 on human CD4+ and CD8+ T cells (FIGS. 20A and 20B, respectively) and proliferation of human CD4+ and CD8+ T cells (FIGS. 20C and 20D, respectively). PBMCs were co-cultured with CEA/MSLN double positive HPAC cells in presence of either CEA×CD3 alone (to trigger T cell activation signal 1) or in combination to either a CEA×CD28 or a MSLN×CD28 bispecific antibody of the invention (providing T cell activation signal 2). The combination of CEA×CD3 and CD28 bsAbs induced a much stronger activation of both CD4+ and CD8+ T cells than the CEA×CD3 monotherapy at the different E:T ratio tested. Similarly, the combination of CEA×CD3 and CD28 bsAbs also resulted in higher percentage of proliferative CD4+ and CD8+ T cells compared to CEA×CD3 monotherapy, at all E:T ratio tested.

FIG. 21 is a schematic diagram of the experimental design of the in vivo efficacy study shown on FIG. 22, where CEA×CD28 bispecific antibody AI3AC84/N was tested in combination with CEA×CD3 in 67 mice subcutaneously engrafted with HPAF-II cells.

FIG. 22A, FIG. 22B are a series of graphs showing in vivo anti-tumor efficacy of the CEA×CD28 bispecific antibody AI3AC84/N in the mouse model described on FIG. 21. The survival proportion at day 56 post engraftment (FIG. 22A) and the growth of tumors in individual mice (FIG. 22B) are shown for all treatment groups. In vivo anti-tumoral activity of CEA×CD3 is enhanced by AI3AC84/N.

### DETAILED DESCRIPTION OF THE INVENTION

The invention is based in part on a bispecific antibody (bsAb) capable of tumor-dependent T cell activation and tumor cell killing. Specifically, the invention is based upon bsAb co-engagement of a tumor associated antigen (TAA) expressed at the surface of the target cells to mediate CD28 clustering and therefore, tumor-specific T cell activation. The invention also provides CD28 antigen binding domains, antigen binding fragments and antibodies.

The bsAbs of the invention are characterized by a single agonist CD28 antigen binding domain for the monovalent co-stimulation of CD28, and a second antigen binding domain capable of binding specifically and monovalently binding to a tumor associated antigen for the targeted delivery of the bsAb to the tumor microenvironment. The bsAbs of the invention are referred to herein as CD28×TAA bsAbs or TAA×CD28 bsAbs.

CD28 is a key co-stimulatory receptor expressed at the surface of T lymphocyte. It belongs to a subfamily of costimulatory molecules characterized by an extracellular variable immunoglobulin-like domain. Other members in the family of molecule include CTLA-4, ICOS, PD-1 and BTLA.

In humans, CD28 is expressed at the cell surface of T lymphocyte as a disulfide-linked homodimer, and is found on approximately 80% of human CD4+ T cells and 50% of CD8+ T cells.

Despite lacking intrinsic enzymatic activity, CD28 engagement by its ligands leads to specific phosphorylation and transcriptional signaling which ultimately results in metabolic changes and in the production of key cytokines, chemokines, and survival signals that are essential for long-term expansion and differentiation of T cells.

The primary ligands for CD28 are CD80 (B7.1) and CD86 (B7.2), which are mainly expressed at the surface of professional antigen presenting cells (APC). CD80 and CD86 diverge in their expression patterns, multimeric states, and functionality. Because CD28 and CTLA-4 are highly homologous, they compete for the same ligands. However, since CTLA-4 binds these ligands with a higher affinity than CD28, CTLA-4 competes with CD28 for ligands and ultimately suppresses T cells responses.

Several anti-CD28 monoclonal antibodies have been proposed for the therapeutic targeting of CD28. A fraction of the identified anti-CD28 antibodies, termed superagonist (SA) antibodies, were found to induce the full activation of primary resting T cells even in the absence of TCR ligation (signal 1), via the clustering of CD28 at the surface of the T cells. The first-in-human study of one of such SA anti-CD28 antibodies, TGN1412, resulted however in severe inflammatory reactions as well as chronic organ failure in all healthy volunteers subjected to the treatment. A cytokine storm predicted by neither in vivo nor in vitro preclinical safety studies was the cause of these adverse events.

To avoid the safety issues linked to SA antibodies or to systemic CD28 co-stimulation, tumor-targeted CD28 bispecific antibodies can be designed to co-stimulate T cells specifically within the tumor microenvironment. By pairing an agonist anti-CD28 binding domain to an anti-TAA binding domain, molecules capable of bridging T cells to malignant cells expressing the selected TAA are generated. Since CD28 bsAbs can only bind to CD28 monovalently, CD28 cannot be successfully clustered in the absence of TAA-positive target cells, thus preventing systemic T cell activation.

Furthermore, even in presence of TAA-positive cancer cells which allow for CD28 clustering at the surface of the T cells, the full cytotoxic potential of T cells can only be unleashed by TAA×CD28 bispecific antibodies in presence of primary T cell stimulation via the TCR. This contrasts with the bivalent superagonist CD28 monoclonal antibodies that were described above. Preclinical studies have shown the benefit of adding costimulatory TAA×CD28 bsAbs for the treatment of solid tumors, boosting the efficacy of bispecific T cell engagers or PD-(L)1 checkpoint inhibitors Examples of agonist TAA×CD28 bsAbs are described in WO2019246514, WO2020198009, WO2020132066, WO2020132024, WO2020127618, WO2021259890, WO2021155071 and WO2022040482, with some of these molecules currently being tested in clinical trials (ClinicalTrials.gov Identifiers: NCT04590326, NCT03972657, NCT04626635).

TAAs are well known in the art and include, for example, a glioma-associated antigen, carcinoembryonic antigen (CEA), EGFRvIII, glypican 3 (GPC3), cMet, IL-IIRa, IL-13Ra, EGFR, FAP, FcRH5, B7H3, Kit, CA LX, CS-1, MUC1, BCMA, bcr-abl, HER2, HER3, β-human chorionic gonadotropin, alphafetoprotein (AFP), ALK, CD19, CD123, lectin-reactive AFP, Fos-related antigen 1, ADRB3, thyroglobulin, EphA2, RAGE-1, RUI, RU2, SSX2, AKAP-4, LCK, OY-TESI, PAXS, SART3, CLL-1, fucosyl GM1, GloboH, MN-CA IX, EPCAM, EVT6-AML, TGSS, polysialic acid, PLAC1, RUI, RU2 (AS), intestinal carboxyl esterase, lewisY, sLe, LY6K, mut hsp70-2, MYCN, RhoC, TRP-2, CYPIBI, BORIS, prostase, prostate-specific antigen (PSA), PAX3, PAP, NY-ESO-1, LAGE-Ia, LMP2, NCAM, Ras mutant, gpIOO, prostein, OR51E2, PANX3, PSMA, PSCA, Her2/neu, hTERT, HMWMAA, HAVCR1, VEGFR2, PDGFR-β, survivin and telomerase, legumain, HPV E6,E7, sperm protein 17, SSEA-4, tyrosinase, TARP, WT1, prostate-carcinoma tumor antigen-1 (PCTA-1), ML-IAP, MAGE, MAGE-A1, MAD-CT-1, MAD-CT-2, MelanA/MART 1, XAGE1, ELF2M, ERG (TMPRSS2 ETS fusion gene), NA17, neutrophil elastase, sarcoma translocation breakpoints, NY-BR-1, ephnnB2, CD20, CD22, CD24, CD30, CD33, CD38, CD44v6, CD97, CD171, CD179a, FAP, IGF-I receptor, GD2, o-acetyl-GD2, GD3, GM3, GPRC5D, GPR20, CXORF61, folate receptor (FRa), folate receptor beta, ROR1, Flt3, TAG72, TN Ag, Tie 2, TEM1, TEM7R, CLDN6, CLDN18.2, TSHR, UPK2, and mesothelin (MSLN).

In specific embodiments the TAA is to CEA, PSMA, EpCAM, GPC3, MSLN, HER2, HER3, or BCMA. In some embodiments, the TAA is CEA. In some embodiments, the TAA is MSLN.

Carcinoembryonic Antigen

Carcinoembryonic antigen (CEA, CEACAM5, CD66e) is a member of the carcinoembryonic antigen-related cell adhesion molecules (CEACAM) family. CEACAMs are involved in a variety of biological processes, including but not limited to cell adhesion, intracellular and intercellular signaling, cancer progression, angiogenesis, and metastasis

CEA was initially discovered as a specific carcinoembryonic antigen of the human gastrointestinal (GI) system. It was found to be highly expressed by GI-tissue during fetal development, but its expression is drastically reduced before birth, and in healthy persons CEA is normally found to be expressed at low level only on the apical side of epithelial cells of the gastrointestinal epithelium and on other mucosal epithelia such as the nasopharynx, the lung, and the urogenital tract. Such polarized and restricted expression is lost during tumorigenesis, and CEA is overexpressed in variety of solid tumors of epithelial origin including colorectal, pancreatic, gastric, non-small cell lung and breast cancers

CEA represents a promising TAA for the development of antibody-based therapies because of the differential expression pattern and expression level observed between cancer cells and normal epithelial cells. The latter only express CEA at the apical surface of the epithelia, a region from which therapeutic antibodies are excluded due to tight junctions.

Multiple monoclonal antibodies targeting CEA have been developed for research purposes, as diagnostic tools, and for therapeutic purposes (see e.g. WO2012117002). In the past years, several bispecific antibodies carrying an anti-CEA arm as targeting arm have been described, including CEA×CD3 bispecific antibodies Cibisatamab ((Bacac et al., 2016) and US20140242079), MEDI-565 ((Oberst et al., 2014) and WO2016036678A1) or those described in WO2021053587, and, more recently, CEA×CD28 bsAbs (e.g. WO2020127628A1 and WO2020127618) and CEA×CD47 bsAbs (e.g. WO2021110647).

Anti-CEA Antibodies

Exemplary anti-CEA antibodies (e.g. “AC22”, “AC61”, “AC84”) of the disclosure are described below. Table 1 shows the amino acid sequences of the antibodies described herein.

In some embodiments, the AC22 antibody has a heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, and a light chain comprising a CDRL1 comprising the amino acid sequence of SEQ ID NO: 8, a CDRL2 comprising the amino acid sequence of SEQ ID NO: 9, and a CDRL3 comprising the amino acid sequence of SEQ ID NO: 10.

In some embodiments, the AC22 antibody has a heavy chain variable region comprising the amino acid sequence of SEQ ID NO: 4, and a light chain variable region comprising the amino acid sequence of SEQ ID NO: 17.

In some embodiments, the AC22 bispecific antibody has a heavy chain comprising the amino acid sequence of SEQ ID NO: 7, and a light chain comprising the amino acid sequence of SEQ ID NO: 20.

In some embodiments, the AC61 antibody has a heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, and a light chain comprising a CDRL1 comprising the amino acid sequence of SEQ ID NO: 11, a CDRL2 comprising the amino acid sequence of SEQ ID NO: 12, and a CDRL3 comprising the amino acid sequence of SEQ ID NO: 13.

In some embodiments, the AC61 antibody has a heavy chain variable region comprising the amino acid sequence of SEQ ID NO: 4, and a light chain variable region comprising the amino acid sequence of SEQ ID NO: 18.

In some embodiments, the AC61 bispecific antibody has a heavy chain comprising the amino acid sequence of SEQ ID NO: 7, and a light chain comprising the amino acid sequence of SEQ ID NO: 21.

In some embodiments, the AC84 antibody has a heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, and a light chain comprising a CDRL1 comprising the amino acid sequence of SEQ ID NO: 14, a CDRL2 comprising the amino acid sequence of SEQ ID NO: 15, and a CDRL3 comprising the amino acid sequence of SEQ ID NO: 16.

In some embodiments, the AC84 antibody has a heavy chain variable region comprising the amino acid sequence of SEQ ID NO: 4, and a light chain variable region comprising the amino acid sequence of SEQ ID NO: 19.

In some embodiments, the AC84 bispecific antibody has a heavy chain comprising the amino acid sequence of SEQ ID NO: 7, and a light chain comprising the amino acid sequence of SEQ ID NO: 22.

Mesothelin

Mesothelin (MSLN) is a glycosylphosphatidylinositol (GPI)-anchored membrane protein expressed in normal tissues at relatively low levels. The mesothelin gene encodes a precursor protein of 628-amino acids that is addressed to the cell membrane, where it is processed into a membrane-bound mature form of 40 kDa, releasing a 31-kDa fragment named megakaryocyte potentiating factor (MPF).

Mesothelin was found to be overexpressed in many solid tumors, including mesothelioma, pancreatic cancer, gastric cancer, ovarian cancer, non-small cell lung cancer, triple-negative breast cancer and prostate cancers. Mesothelin was suggested to play a role in cancer cell proliferation, local invasion, and tumor metastasis, and its expression on tumor cells has been correlated with increased tumor aggressiveness and poor clinical outcome.

Because of the differential expression observed between cancer cells and healthy tissues, mesothelin represent a favorable TAA for the development of therapeutic agents targeting mesothelin, some of which are currently undergoing clinical trials.

Anti-MSLN Antibodies

Exemplary anti-MSLN antibodies (e.g. “O30”, “O25”, “O35”, “O38”, “O41”) of the disclosure are described below. Table 1 shows the amino acid sequences of the antibodies described herein.

In some embodiments, the O30 antibody has a heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, and a light chain comprising a CDRL1 comprising the amino acid sequence of SEQ ID NO: 128, a CDRL2 comprising the amino acid sequence of SEQ ID NO: 129, and a CDRL3 comprising the amino acid sequence of SEQ ID NO: 130.

In some embodiments, the O30 antibody has a heavy chain variable region comprising the amino acid sequence of SEQ ID NO: 4, and a light chain variable region comprising the amino acid sequence of SEQ ID NO: 143.

In some embodiments, the O30 bispecific antibody has a heavy chain comprising the amino acid sequence of SEQ ID NO: 7, and a light chain comprising the amino acid sequence of SEQ ID NO: 148.

In some embodiments, the O25 antibody has a heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, and a light chain comprising a CDRL1 comprising the amino acid sequence of SEQ ID NO: 131, a CDRL2 comprising the amino acid sequence of SEQ ID NO: 132, and a CDRL3 comprising the amino acid sequence of SEQ ID NO: 133.

In some embodiments, the O25 antibody has a heavy chain variable region comprising the amino acid sequence of SEQ ID NO: 4, and a light chain variable region comprising the amino acid sequence of SEQ ID NO: 144.

In some embodiments, the O25 bispecific antibody has a heavy chain comprising the amino acid sequence of SEQ ID NO: 7, and a light chain comprising the amino acid sequence of SEQ ID NO: 149.

In some embodiments, the O35 antibody has a heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, and a light chain comprising a CDRL1 comprising the amino acid sequence of SEQ ID NO: 134, a CDRL2 comprising the amino acid sequence of SEQ ID NO: 135, and a CDRL3 comprising the amino acid sequence of SEQ ID NO: 136.

In some embodiments, the O35 antibody has a heavy chain variable region comprising the amino acid sequence of SEQ ID NO: 4, and a light chain variable region comprising the amino acid sequence of SEQ ID NO: 145.

In some embodiments, the O35 bispecific antibody has a heavy chain comprising the amino acid sequence of SEQ ID NO: 7, and a light chain comprising the amino acid sequence of SEQ ID NO: 150.

In some embodiments, the O38 antibody has a heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, and a light chain comprising a CDRL1 comprising the amino acid sequence of SEQ ID NO: 137, a CDRL2 comprising the amino acid sequence of SEQ ID NO: 138, and a CDRL3 comprising the amino acid sequence of SEQ ID NO: 139.

In some embodiments, the O38 antibody has a heavy chain variable region comprising the amino acid sequence of SEQ ID NO: 4, and a light chain variable region comprising the amino acid sequence of SEQ ID NO: 146.

In some embodiments, the O38 bispecific antibody has a heavy chain comprising the amino acid sequence of SEQ ID NO: 7, and a light chain comprising the amino acid sequence of SEQ ID NO: 151.

In some embodiments, the O41 antibody has a heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, and a light chain comprising a CDRL1 comprising the amino acid sequence of SEQ ID NO: 140, a CDRL2 comprising the amino acid sequence of SEQ ID NO: 141, and a CDRL3 comprising the amino acid sequence of SEQ ID NO: 142.

In some embodiments, the O41 antibody has a heavy chain variable region comprising the amino acid sequence of SEQ ID NO: 4, and a light chain variable region comprising the amino acid sequence of SEQ ID NO: 147.

In some embodiments, the O41 bispecific antibody has a heavy chain comprising the amino acid sequence of SEQ ID NO: 7, and a light chain comprising the amino acid sequence of SEQ ID NO: 152.

Bispecific Antibodies

The bsAbs antibodies according to the invention may be generated de novo or may be engineered from existing monospecific CD28 antibodies and TAA antibodies. The bispecific antibodies of the invention have one antigen binding region that is specific for CD28 and a second antigen binding region that is specific for a TAA. In some embodiments, the TAA is CEA. In some embodiments, the TAA is MSLN. In some aspects, the bispecific antibodies are monovalent for CD28, CEA and MSLN.

The bsAbs of the invention can be based on any of the different antibody formats that have been previously described. In general, IgG-like formats are preferred as they provide favorable properties such as long half-life and potentially reduced immunogenicity, but any other molecular bispecific format can also be used for the invention. In some embodiments, the bispecific antibodies share a common heavy chain. In some embodiments, the heavy chains are native heavy chains (i.e, does not contain any mutations (“wildtype”)). In some embodiments, the heavy chains comprise at least one mutation (i.e, with “LALA” mutation or “LALAPA” mutation). Optionally, the bispecific antibodies have light chains of different types. For example, one light chain is a kappa light and the other light chain is a lambda light chain (i.e., kl-body) Differing light chains allows the bispecific to be purified easily using kappa and lambda select resins.

The heavy and light chain amino acid sequences of the antibodies identified by their United States Adopted Names (USAN are available for example via the American Medical Association at https://www.ama-assn.org/ or via the CAS registry).

Monospecific CD28 and TAA binding variable domains may be selected de novo from for example a phage display library, where the phage is engineered to express human immunoglobulins or portions thereof such as Fabs, single chain variable fragments (scFv), or unpaired or paired antibody variable regions and subsequently engineered into a bispecific format. The CD28 and TAA variable domains can be isolated for example from phage display libraries expressing antibody heavy and light chain variable regions as fusion proteins with bacteriophage g3 protein (scFv-g3 fusion proteins).

The antibody libraries are screened for binding CD28 antibodies and TAA and the obtained positive clones are further characterized. Such phage display methods for isolating human antibodies are established in the art. See for example: U.S. Pat. Nos. 5,223,409; 5,403,484; and 5,571,698, 5,427,908, 5,580,717, 5,969,108, 6,172,197, 5,885,793; 6,521,404; 6,544,731; 6,555,313; 6,582,915 and 6,593,081. The obtained de novo variable regions binding are engineered to bispecific formats using the methods know in the art and described herein.

Additionally, bispecific antibodies of the invention can be made using the techniques, including those disclosed in WO 2012/023053, filed Aug. 16, 2011, the contents of which are hereby incorporated by reference in their entirety. The methods described in WO 2012/023053 generate bispecific antibodies that are identical in structure to a human immunoglobulin. This type of molecule is composed of two copies of a unique heavy chain polypeptide, a first light chain variable region fused to a constant Kappa domain and second light chain variable region fused to a constant Lambda domain. Each combining site displays a different antigen specificity to which both the heavy and light chain contribute. The light chain variable regions can be of the Lambda or Kappa family and are preferably fused to a Lambda and Kappa constant domains, respectively. This is preferred in order to avoid the generation of non-natural polypeptide junctions.

However, it is also possible to obtain bispecific antibodies of the invention by fusing a Kappa light chain variable domain to a constant Lambda domain for a first specificity and fusing a Lambda light chain variable domain to a constant Kappa domain for the second specificity. The bispecific antibodies described in WO 2012/023053 are referred to as IgGκλ antibodies or “κλ bodies,” a new fully human bispecific IgG format. This κλ-body format allows the affinity purification of a bispecific antibody that is undistinguishable from a standard IgG molecule with characteristics that are undistinguishable from a standard monoclonal antibody and, therefore, favorable as compared to previous formats.

In addition to methods described above, bispecific antibodies of the invention can be generated in vitro in a cell-free environment by introducing asymmetrical mutations in the CH3 regions of two monospecific homodimeric antibodies and forming the bispecific heterodimeric antibody from two parent monospecific homodimeric antibodies in reducing conditions to allow disulfide bond isomerization according to methods described in Intl. Pat. Publ. No. WO2011/131746. In the methods, the first monospecific bivalent antibody and the second monospecific bivalent antibody are engineered to have certain substitutions at the CH3 domain that promoter heterodimer stability; the antibodies are incubated together under reducing conditions sufficient to allow the cysteines in the hinge region to undergo disulfide bond isomerization; thereby generating the bispecific antibody by Fab arm exchange.

Antibodies of the present invention have two or more antigen binding domains and are bispecific. Bispecific antibodies of the invention include antibodies having a full length antibody structure or partial length antibody structure such as Fab

“Full length antibody” as used herein refers to an antibody having two full length antibody heavy chains and two full length antibody light chains. A full length antibody heavy chain (HC) consists of well known heavy chain variable and constant domains VH, CH1, CH2, and CH3. A full length antibody light chain (LC) consists of well known light chain variable and constant domains VL and CL. The full length antibody may be lacking the C-terminal lysine (K) in either one or both heavy chains.

The term “Fab-arm” or “half molecule” refers to one heavy chain-light chain pair that specifically binds an antigen.

Full length bispecific antibodies of the invention may be generated for example using Fab arm exchange (or half molecule exchange) between two monospecific bivalent antibodies by introducing substitutions at the heavy chain CH3 interface in each half molecule to favor heterodimer formation of two antibody half molecules having distinct specificity either in vitro in cell-free environment or using co-expression. The Fab arm exchange reaction is the result of a disulfide-bond isomerization reaction and dissociation-association of CH3 domains. The heavy-chain disulfide bonds in the hinge regions of the parent monospecific antibodies are reduced. The resulting free cysteines of one of the parent monospecific antibodies form an inter heavy-chain disulfide bond with cysteine residues of a second parent monospecific antibody molecule and simultaneously CH3 domains of the parent antibodies release and reform by dissociation-association. The CH3 domains of the Fab arms may be engineered to favor heterodimerization over homodimerization. The resulting product is a bispecific antibody having two Fab arms or half molecules which each bind a distinct epitope.

“Homodimerization” as used herein refers to an interaction of two heavy chains having identical CH3 amino acid sequences. “Homodimer” as used herein refers to an antibody having two heavy chains with identical CH3 amino acid sequences.

“Heterodimerization” as used herein refers to an interaction of two heavy chains having non-identical CH3 amino acid sequences. “Heterodimer” as used herein refers to an antibody having two heavy chains with non-identical CH3 amino acid sequences.

The “knob-in-hole” strategy (see, e.g., PCT Intl. Publ. No. WO 2006/028936) may be used to generate full length bispecific antibodies. Briefly, selected amino acids forming the interface of the CH3 domains in human IgG can be mutated at positions affecting CH3 domain interactions to promote heterodimer formation. An amino acid with a small side chain (hole) is introduced into a heavy chain of an antibody specifically binding a first antigen and an amino acid with a large side chain (knob) is introduced into a heavy chain of an antibody specifically binding a second antigen. After co-expression of the two antibodies, a heterodimer is formed as a result of the preferential interaction of the heavy chain with a “hole” with the heavy chain with a “knob”. Exemplary CH3 substitution pairs forming a knob and a hole are (expressed as modified position in the first CH3 domain of the first heavy chain/modified position in the second CH3 domain of the second heavy chain): T366Y/F405A, T366W/F405W, F405W/Y407A, T394W/Y407T, T394S/Y407A, T366W/T394S, F405W/T394S and T366W/T366S_L368A_Y407V.

Other strategies such as promoting heavy chain heterodimerization using electrostatic interactions by substituting positively charged residues at one CH3 surface and negatively charged residues at a second CH3 surface may be used, as described in US Pat. Publ. No. US2010/0015133; US Pat. Publ. No. US2009/0182127; US Pat. Publ. No. US2010/028637 or US Pat. Publ. No. US2011/0123532. In other strategies, heterodimerization may be promoted by following substitutions (expressed as modified position in the first CH3 domain of the first heavy chain/modified position in the second CH3 domain of the second heavy chain): L351Y_F405A_Y407V/T394W, T366I_K392M_T394W/F405A_Y407V, T366L_K392M_T394W/F405A_Y407V, L351Y_Y407A/T366A_K409F, L351Y_Y407A/T366V_K409F, Y407A/T366A_K409F, or T350V_L351Y_F405A_Y407V/T350V_T366L_K392L_T394W as described in U.S. Pat. Publ. No. US2012/0149876 or U.S. Pat. Publ. No. US2013/0195849

Exemplary anti-cell surface antibodies that may be used to engineer bispecific molecules include for example anti-tumor associate antigen antibodies know in the art, such as Pertuzumab and Trastuzumab (HER-2); Cetuximab, Necitumumab, Panitumumab and Amivantamab (EGFR); Labetuzumab and Cibisatamab (CEA); Amatuximab (mesothelin); Cordrituzumab (glypican 3); Atezolizumab, Avelumab and Durvalumab (PD-L1); Blinatumomab (CD19); Brentuximab (CD30); Daratumumab (CD38); Gemtuzumab (CD33); Tositumomab 9CD22) or Obinutuzumab, Ocrelizumab, Ofatumumab, Rituximab, and Ibritumomab (CD2H).

Exemplary CD28, CEA and MSLN antibodies that may be used to engineer bispecific molecules include the antibodies disclosed herein. Exemplary anti-CD28 antibodies from which the CD28 antigen binding region can be derived from include the “AI3”, “AI5”, “AI7”, “AI8”, “AI9”, “AI10”, “AI11”, “AI12”, “AI13”, “AI14”, “AI15”, “AI16”, “AI17”, “AI18”, “AI19”, “AI20”, “AI21”, “AI22” or “AI23” antibody. Exemplary anti-CEA antibodies from which the CEA antigen binding region can be derived from include the “AC22”, “AC61” or “AC84” antibody. Exemplary anti-MSLN antibodies from which the MSLN antigen binding region can be derived from include the “O30”, “O25”, “O35”, “O38” or “O41” antibody. Table 1 shows the amino acid sequences of the regions of the antibodies of the disclosure.

TABLE 1
Exemplary Amino Acid Sequences of the Disclosure

| SEQ ID | | |
|---|---|---|
| NO: | Name | Amino Acid Sequence |
| SEQ ID | Common heavy chain | GFTFSSYA |
| NO: 1 | CDR-H1 | |
| SEQ ID | Common heavy chain | ISGSGGST |
| NO: 2 | CDR-H2 | |
| SEQ ID | Common heavy chain | AKSYGAFDY |
| NO: 3 | CDR-H3 | |
| SEQ ID | Common heavy chain | EVQLLESGGGLVQPGGSLRLSCAASGFTFSSYAMSWVR |
| NO: 4 | VH | QAPGKGLEWVSAISGSGGSTYYADSVKGRFTISRDNSK |
| | | NTLYLQMNSLRAEDTAVYYCAKSYGAFDYWGQGTLVTV |
| | | SS |
| SEQ ID | Common heavy chain | EVQLLESGGGLVQPGGSLRLSCAASGFTFSSYAMSWVR |
| NO: 5 | HC (wild-type) | QAPGKGLEWVSAISGSGGSTYYADSVKGRFTISRDNSK |
| | | NTLYLQMNSLRAEDTAVYYCAKSYGAFDYWGQGTLVTV |
| | | SSASTKGPSVFPLAPSSKSTSGGTAALGCLVKDYFPEP |
| | | VTVSWNSGALTSGVHTFPAVLQSSGLYSLSSVVTVPSS |
| | | SLGTQTYICNVNHKPSNTKVDKRVEPKSCDKTHTCPPC |
| | | PAPELLGGPSVFLFPPKPKDTLMISRTPEVTCVVVDVS |
| | | HEDPEVKFNWYVDGVEVHNAKTKPREEQYNSTYRVVSV |
| | | LTVLHQDWLNGKEYKCKVSNKALPAPIEKTISKAKGQP |
| | | REPQVYTLPPSREEMTKNQVSLTCLVKGFYPSDIAVEW |
| | | ESNGQPENNYKTTPPVLDSDGSFFLYSKLTVDKSRWQQ |
| | | GNVFSCSVMHEALHNHYTQKSLSLSPG |
| SEQ ID | Common heavy chain | EVQLLESGGGLVQPGGSLRLSCAASGFTFSSYAMSWVR |
| NO: 6 | HC (with LALA | QAPGKGLEWVSAISGSGGSTYYADSVKGRFTISRDNSK |
| | mutation) | NTLYLQMNSLRAEDTAVYYCAKSYGAFDYWGQGTLVTV |
| | | SSASTKGPSVFPLAPSSKSTSGGTAALGCLVKDYFPEP |
| | | VTVSWNSGALTSGVHTFPAVLQSSGLYSLSSVVTVPSS |
| | | SLGTQTYICNVNHKPSNTKVDKRVEPKSCDKTHTCPPC |
| | | PAPEAAGGPSVFLFPPKPKDTLMISRTPEVTCVVVDVS |
| | | HEDPEVKFNWYVDGVEVHNAKTKPREEQYNSTYRVVSV |
| | | LTVLHQDWLNGKEYKCKVSNKALPAPIEKTISKAKGQP |
| | | REPQVYTLPPSREEMTKNQVSLTCLVKGFYPSDIAVEW |
| | | ESNGQPENNYKTTPPVLDSDGSFFLYSKLTVDKSRWQQ |
| | | GNVFSCSVMHEALHNHYTQKSLSLSPG |
| SEQ ID | Common heavy chain | EVQLLESGGGLVQPGGSLRLSCAASGFTFSSYAMSWVR |
| NO: 7 | HC (with LALAPA | QAPGKGLEWVSAISGSGGSTYYADSVKGRFTISRDNSK |
| | mutation) | NTLYLQMNSLRAEDTAVYYCAKSYGAFDYWGQGTLVTV |
| | | SSASTKGPSVFPLAPSSKSTSGGTAALGCLVKDYFPEP |
| | | VTVSWNSGALTSGVHTFPAVLQSSGLYSLSSVVTVPSS |
| | | SLGTQTYICNVNHKPSNTKVDKRVEPKSCDKTHTCPPC |
| | | PAPEAAGGPSVFLFPPKPKDTLMISRTPEVTCVVVDVS |
| | | HEDPEVKFNWYVDGVEVHNAKTKPREEQYNSTYRVVSV |
| | | LTVLHQDWLNGKEYKCKVSNKALAAPIEKTISKAKGQP |
| | | REPQVYTLPPSREEMTKNQVSLTCLVKGFYPSDIAVEW |
| | | ESNGQPENNYKTTPPVLDSDGSFFLYSKLTVDKSRWQQ |
| | | GNVFSCSVMHEALHNHYTQKSLSLSPG |
| SEQ ID | CEA binding part | SSNIANGI |
| NO: 8 | AC22 CDR-L1 | |
| SEQ ID | CEA binding part | FDN |
| NO: 9 | AC22 CDR-L2 | |
| SEQ ID | CEA binding part | GTWDFSYGIV |
| NO: 10 | AC22 CDR-L3 | |
| SEQ ID | CEA binding part | QNIPRT |
| NO: 11 | AC61 CDR-L1 | |
| SEQ ID | CEA binding part | DIS |
| NO: 12 | AC61 CDR-L2 | |
| SEQ ID | CEA binding part | QQVTLDPEEMT |
| NO: 13 | AC61 CDR-L3 | |
| SEQ ID | CEA binding part | SSNIGIGL |
| NO: 14 | AC84 CDR-L1 | |
| SEQ ID | CEA binding part | NVN |
| NO: 15 | AC84 CDR-L2 | |
| SEQ ID | CEA binding part | GTWDFSYRVD |
| NO: 16 | AC84 CDR-L3 | |
| SEQ ID | CEA binding part | QSVLTQPPSVSAAPGQKVTISCSGSSSNIANGIVSWYQ |
| NO: 17 | AC22 VL (light chain | QLPGTAPKLLIYFDNLRPSGIPDRFSGSKSGTSATLGI |
| | variable region) | TGLQTGDEADYYCGTWDFSYGIVFGGGTKLTVL |
| SEQ ID | CEA binding part | DIQMTQSPSSLSASVGDRVTITCQASQNIPRTINWYQQ |
| NO: 18 | AC61 VL (light chain | KPGKAPKLLIYDISNLETGVPSRFSGSGSGTDFTFTIS |
| | variable region) | SLQPEDIATYYCQQVTLDPEEMTFGQGTKVEIK |
| SEQ ID | CEA binding part | QSVLTQPPSVSAAPGQKVTISCSGSSSNIGIGLVSWYQ |
| NO: 19 | AC84 VL (light chain | QLPGTAPKLLIYNVNTRPSGIPDRFSGSKSGTSATLGI |
| | variable region) | TGLQTGDEADYYCGTWDFSYRVDFGGGTKLTVL |
| SEQ ID | CEA binding part | QSVLTQPPSVSAAPGQKVTISCSGSSSNIANGIVSWYQ |
| NO: 20 | AC22 LC (light | QLPGTAPKLLIYFDNLRPSGIPDRFSGSKSGTSATLGI |
| | chain) | TGLQTGDEADYYCGTWDFSYGIVFGGGTKLTVLGQPKA |
| | | APSVTLFPPSSEELQANKATLVCLISDFYPGAVTVAWK |
| | | ADSSPVKAGVETTTPSKQSNNKYAASSYLSLTPEQWKS |
| | | HRSYSCQVTHEGSTVEKTVAPTECS |
| SEQ ID | CEA binding part | DIQMTQSPSSLSASVGDRVTITCQASQNIPRTINWYQQ |
| NO: 21 | AC61 LC (light | KPGKAPKLLIYDISNLETGVPSRFSGSGSGTDFTFTIS |
| | chain) | SLQPEDIATYYCQQVTLDPEEMTFGQGTKVEIKRTVAA |
| | | PSVFIFPPSDEQLKSGTASVVCLLNNFYPREAKVQWKV |
| | | DNALQSGNSQESVTEQDSKDSTYSLSSTLTLSKADYEK |
| | | HKVYACEVTHQGLSSPVTKSFNRGEC |
| SEQ ID | CEA binding part | QSVLTQPPSVSAAPGQKVTISCSGSSSNIGIGLVSWYQ |
| NO: 22 | AC84 LC (light | QLPGTAPKLLIYNVNTRPSGIPDRFSGSKSGTSATLGI |
| | chain) | TGLQTGDEADYYCGTWDFSYRVDFGGGTKLTVLGQPKA |
| | | APSVTLFPPSSEELQANKATLVCLISDFYPGAVTVAWK |
| | | ADSSPVKAGVETTTPSKQSNNKYAASSYLSLTPEQWKS |
| | | HRSYSCQVTHEGSTVEKTVAPTECS |
| SEQ ID | CD28 binding part | QSVLYSSNNKNY |
| NO: 23 | AI3 CDR-L1 | |
| SEQ ID | CD28 binding part | WAS |
| NO: 24 | AI3 CDR-L2 | |
| SEQ ID | CD28 binding part | QQNLRPPET |
| NO: 25 | AI3 CDR-L3 | |
| SEQ ID | CD28 binding part | GDLLTFEGKTY |
| NO: 26 | AI5 CDR-L1 | |
| SEQ ID | CD28 binding part | EVS |
| NO: 27 | AI5 CDR-L2 | |
| SEQ ID | CD28 binding part | MQAHSTRFAFT |
| NO: 28 | AI5 CDR-L3 | |
| SEQ ID | CD28 binding part | SSNIGSNT |
| NO: 29 | AI7 CDR-L1 | |
| SEQ ID | CD28 binding part | SEN |
| NO: 30 | AI7 CDR-L2 | |
| SEQ ID | CD28 binding part | AAWDLNPAIGYV |
| NO: 31 | AI7 CDR-L3 | |
| SEQ ID | CD28 binding part | DDLLRFSGKTY |
| NO: 32 | AI8 CDR-L1 | |
| SEQ ID | CD28 binding part | EVS |
| NO: 33 | AI8 CDR-L2 | |
| SEQ ID | CD28 binding part | MQASRSKVAFT |
| NO: 34 | AI8 CDR-L3 | |
| SEQ ID | CD28 binding part | GDLLSFDGKTY |
| NO: 35 | AI9 CDR-L1 | |
| SEQ ID | CD28 binding part | EVS |
| NO: 36 | AI9 CDR-L2 | |
| SEQ ID | CD28 binding part | MQASKHRFGFT |
| NO: 37 | AI9 CDR-L3 | |
| SEQ ID | CD28 binding part | GDLLEFAGKTY |
| NO: 38 | AI10 CDR-L1 | |
| SEQ ID | CD28 binding part | EVS |
| NO: 39 | AI10 CDR-L2 | |
| SEQ ID | CD28 binding part | MQAHGSKIGFT |
| NO: 40 | AI10 CDR-L3 | |
| SEQ ID | CD28 binding part | QEIHSH |
| NO: 41 | AI11 CDR-L1 | |
| SEQ ID | CD28 binding part | STS |
| NO: 42 | AI11 CDR-L2 | |
| SEQ ID | CD28 binding part | QQGFRFAFST |
| NO: 43 | AI11 CDR-L3 | |
| SEQ ID | CD28 binding part | QSILYSSSNRNY |
| NO: 44 | AI12 CDR-L1 | |
| SEQ ID | CD28 binding part | WAS |
| NO: 45 | AI12 CDR-L2 | |
| SEQ ID | CD28 binding part | QQNFRPPET |
| NO: 46 | AI12 CDR-L3 | |
| SEQ ID | CD28 binding part | QSVLYSSNNKNY |
| NO: 47 | AI13 CDR-L1 | |
| SEQ ID | CD28 binding part | WAS |
| NO: 48 | AI13 CDR-L2 | |
| SEQ ID | CD28 binding part | QQNFRPPET |
| NO: 49 | AI13 CDR-L3 | |
| SEQ ID | CD28 binding part | QAVFSLD |
| NO: 50 | AI14 CDR-L1 | |
| SEQ ID | CD28 binding part | STS |
| NO: 51 | AI14 CDR-L2 | |
| SEQ ID | CD28 binding part | QQGPAFKPDVT |
| NO: 52 | AI14 CDR-L3 | |
| SEQ ID | CD28 binding part | QSVSSY |
| NO: 53 | AI15 CDR-L1 | |
| SEQ ID | CD28 binding part | DAS |
| NO: 54 | AI15 CDR-L2 | |
| SEQ ID | CD28 binding part | HQRYRPPET |
| NO: 55 | AI15 CDR-L3 | |
| SEQ ID | CD28 binding part | QSIGAY |
| NO: 56 | AI16 CDR-L1 | |
| SEQ ID | CD28 binding part | HAS |
| NO: 57 | AI16 CDR-L2 | |
| SEQ ID | CD28 binding part | QQYKDRPLT |
| NO: 58 | AI16 CDR-L3 | |
| SEQ ID | CD28 binding part | GNLLTHDGKTY |
| NO: 59 | AI17 CDR-L1 | |
| SEQ ID | CD28 binding part | EVS |
| NO: 60 | AI17 CDR-L2 | |
| SEQ ID | CD28 binding part | MQANALKFGFT |
| NO: 61 | AI17 CDR-L3 | |
| SEQ ID | CD28 binding part | SDLLRFEGKTY |
| NO: 62 | AI18 CDR-L1 | |
| SEQ ID | CD28 binding part | EVS |
| NO: 63 | AI18 CDR-L2 | |
| SEQ ID | CD28 binding part | MQAHASALKFT |
| NO: 64 | AI18 CDR-L3 | |
| SEQ ID | CD28 binding part | GNLLTHGGKTY |
| NO: 65 | AI19 CDR-L1 | |
| SEQ ID | CD28 binding part | EVS |
| NO: 66 | AI19 CDR-L2 | |
| SEQ ID | CD28 binding part | MQAHADGFGFT |
| NO: 67 | AI19 CDR-L3 | |
| SEQ ID | CD28 binding part | GDLLRFDGKTY |
| NO: 68 | AI20 CDR-L1 | |
| SEQ ID | CD28 binding part | EVS |
| NO: 69 | AI20 CDR-L2 | |
| SEQ ID | CD28 binding part | MQASETAVRFT |
| NO: 70 | AI20 CDR-L3 | |
| SEQ ID | CD28 binding part | TDLLTFEGKTY |
| NO: 71 | AI21 CDR-L1 | |
| SEQ ID | CD28 binding part | EVS |
| NO: 72 | AI21 CDR-L2 | |
| SEQ ID | CD28 binding part | MQAHSSKFKFT |
| NO: 73 | AI21 CDR-L3 | |
| SEQ ID | CD28 binding part | GDLLSTDGKTY |
| NO: 74 | AI22 CDR-L1 | |
| SEQ ID | CD28 binding part | EVS |
| NO: 75 | AI22 CDR-L2 | |
| SEQ ID | CD28 binding part | MQASGSRFYFT |
| NO: 76 | AI22 CDR-L3 | |
| SEQ ID | CD28 binding part | QTLLYKYDNKNY |
| NO: 77 | AI23 CDR-L1 | |
| SEQ ID | CD28 binding part | WAS |
| NO: 78 | AI23 CDR-L2 | |
| SEQ ID | CD28 binding part | HQYLYPAET |
| NO: 79 | AI23 CDR-L3 | |
| SEQ ID | CD28 binding part | DIVMTQSPDSLAVSLGERATINCKSSQSVLYSSNNKNY |
| NO: 80 | AI3 VL (light chain | LAWYQQKPGQPPKLLIYWASTRESGVPDRFSGSGSGTD |
| | variable region) | FTLTISSLQAEDVAVYYCQQNLRPPETFGQGTKVEIK |
| SEQ ID | CD28 binding part | DIVMTQTPLSLSVTPGQPASISCKSSGDLLTFEGKTYL |
| NO: 81 | AI5 VL (light chain | YWYLQKPGQPPQLLIYEVSNRFSGVPDRFSGSGSGTDF |
| | variable region) | TLKISRVEAEDVGVYYCMQAHSTRFAFTFGQGTKVEIK |
| SEQ ID | CD28 binding part | QSVLTQPPSASGTPGQRVTISCSGSSSNIGSNTVNWYQ |
| NO: 82 | AI7 VL (light chain | QLPGTAPKLLIYSENTRPSGVPDRFSGSKSGTSASLAI |
| | variable region) | SGLQSEDEADYYCAAWDLNPAIGYVFGGGTKLTVL |
| SEQ ID | CD28 binding part | DIVMTQTPLSLSVTPGQPASISCKSSDDLLRFSGKTYL |
| NO: 83 | AI8 VL (light chain | YWYLQKPGQPPQLLIYEVSNRFSGVPDRFSGSGSGTDF |
| | variable region) | TLKISRVEAEDVGVYYCMQASRSKVAFTFGQGTKVEIK |
| SEQ ID | CD28 binding part | DIVMTQTPLSLSVTPGQPASISCKSSGDLLSFDGKTYL |
| NO: 84 | AI9 VL (light chain | YWYLQKPGQPPQLLIYEVSNRFSGVPDRFSGSGSGTDF |
| | variable region) | TLKISRVEAEDVGVYYCMQASKHRFGFTFGQGTKVEIK |
| SEQ ID | CD28 binding part | DIVMTQTPLSLSVTPGQPASISCKSSGDLLEFAGKTYL |
| NO: 85 | AI10 VL (light chain | YWYLQKPGQPPQLLIYEVSNRFSGVPDRFSGSGSGTDF |
| | variable region) | TLKISRVEAEDVGVYYCMQAHGSKIGFTFGQGTKVEIK |
| SEQ ID | CD28 binding part | DIQMTQSPSTLSASVGDRVTITCRASQEIHSHLSWYQQ |
| NO: 86 | AI11 VL (light chain | KPGKAPKLLIYSTSTLESGVPSRFSGSGSGTEFTLTIS |
| | variable region) | SLQPDDFATYYCQQGFRFAFSTFGQGTKVEIK |
| SEQ ID | CD28 binding part | DIVMTQSPDSLGVSLGERATINCKSSQSILYSSSNRNY |
| NO: 87 | AI12 VL (light chain | LAWYQQKPGQPPKLLIYWASTRESGVPDRFSGSGSGTD |
| | variable region) | FTLTISSLQAEDVGVYYCQQNFRPPETFGGGTKVEIK |
| SEQ ID | CD28 binding part | DIVMTQSPDSLAVSLGERATINCKSSQSVLYSSNNKNY |
| NO: 88 | AI13 VL (light chain | LAWYQQKPGQPPKLLIYWASTRESGVPDRFSGSGSGTD |
| | variable region) | FTLTISSLQAEDVGVYYCQQNFRPPETFGGGTKVEIK |
| SEQ ID | CD28 binding part | EIVLTQSPGTLSLSPGERATLSCRASQAVFSLDLHWYQ |
| NO: 89 | AI14 VL (light chain | QKPGQAPRLLIYSTSKRATGIPDRFSGSGSGTDFTLTI |
| | variable region) | SRLEPEDFAVYYCQQGPAFKPDVTFGQGTKVEIK |
| SEQ ID | CD28 binding part | EIVLTQSPATLSLSPGERATLSCRASQSVSSYLAWYQQ |
| NO: 90 | AI15 VL (light chain | KPGQAPRLLIYDASNRATGIPARFSGSGSGTDFTLTIS |
| | variable region) | SLEPEDFAVYYCHQRYRPPETFGQGTKVEIK |
| SEQ ID | CD28 binding part | DIQMTQSPSSLSASVGDRVTITCRASQSIGAYLNWYQQ |
| NO: 91 | AI16 VL (light chain | KPGKAPKLLIYHASDLQSGVPSRFSGSGSGTDFTLTIS |
| | variable region) | SLQPEDFATYYCQQYKDRPLTFGQGTKVEIK |
| SEQ ID | CD28 binding part | DIVMTQTPLSLSVTPGQPASISCKSSGNLLTHDGKTYL |
| NO: 92 | AI17 VL (light chain | YWYLQKPGQPPQLLIYEVSNRFSGVPDRFSGSGSGTDF |
| | variable region) | TLKISRVEAEDVGVYYCMQANALKFGFTFGQGTKVEIK |
| SEQ ID | CD28 binding part | DIVMTQTPLSLSVTPGQPASISCKSSSDLLRFEGKTYL |
| NO: 93 | AI18 VL (light chain | YWYLQKPGQPPQLLIYEVSNRFSGVPDRFSGSGSGTDF |
| | variable region) | TLKISRVEAEDVGVYYCMQAHASALKFTFGQGTKVEIK |
| SEQ ID | CD28 binding part | DIVMTQTPLSLSVTPGQPASISCKSSGNLLTHGGKTYL |
| NO: 94 | AI19 VL (light chain | YWYLQKPGQPPQLLIYEVSNRFSGVPDRFSGSGSGTDF |
| | variable region) | TLKISRVEAEDVGVYYCMQAHADGFGFTFGQGTKVEIK |
| SEQ ID | CD28 binding part | DIVMTQTPLSLSVTPGQPASISCKSSGDLLRFDGKTYL |
| NO: 95 | AI20 VL (light chain | YWYLQKPGQPPQLLIYEVSNRFSGVPDRFSGSGSGTDF |
| | variable region) | TLKISRVEAEDVGVYYCMQASETAVRFTFGQGTKVEIK |
| SEQ ID | CD28 binding part | DIVMTQTPLSLSVTPGQPASISCKSSTDLLTFEGKTYL |
| NO: 96 | AI21 VL (light chain | YWYLQKPGQPPQLLIYEVSNRFSGVPDRFSGSGSGTDF |
| | variable region) | TLKISRVEAEDVGVYYCMQAHSSKFKFTFGQGTKVEIK |
| SEQ ID | CD28 binding part | DIVMTQTPLSLSVTPGQPASISCKSSGDLLSTDGKTYL |
| NO: 97 | AI22 VL (light chain | YWYLQKPGQPPQLLIYEVSNRFSGVPDRFSGSGSGTDF |
| | variable region) | TLKISRVEAEDVGVYYCMQASGSRFYFTFGQGTKVEIK |
| SEQ ID | CD28 binding part | DIVMTQSPDSLAVSLGERAIINCKSSQTLLYKYDNKNY |
| NO: 98 | AI23 VL (light chain | LAWYQHKPGQPPKLLFYWASTRESGVPDRFSGSGSGTD |
| | variable region) | FTLTINSLQAEDVAVYYCHQYLYPAETFGQGTKLEIK |
| SEQ ID | CD28 binding part | DIVMTQSPDSLAVSLGERATINCKSSQSVLYSSNNKNY |
| NO: 99 | AI3 LC (light chain) | LAWYQQKPGQPPKLLIYWASTRESGVPDRFSGSGSGTD |
| | | FTLTISSLQAEDVAVYYCQQNLRPPETFGQGTKVEIKR |
| | | TVAAPSVFIFPPSDEQLKSGTASVVCLLNNFYPREAKV |
| | | QWKVDNALQSGNSQESVTEQDSKDSTYSLSSTLTLSKA |
| | | DYEKHKVYACEVTHQGLSSPVTKSFNRGEC |
| SEQ ID | CD28 binding part | DIVMTQTPLSLSVTPGQPASISCKSSGDLLTFEGKTYL |
| NO: 100 | AI5 LC (light chain) | YWYLQKPGQPPQLLIYEVSNRFSGVPDRFSGSGSGTDF |
| | | TLKISRVEAEDVGVYYCMQAHSTRFAFTFGQGTKVEIK |
| | | RTVAAPSVFIFPPSDEQLKSGTASVVCLLNNFYPREAK |
| | | VQWKVDNALQSGNSQESVTEQDSKDSTYSLSSTLTLSK |
| | | ADYEKHKVYACEVTHQGLSSPVTKSFNRGEC |
| SEQ ID | CD28 binding part | QSVLTQPPSASGTPGQRVTISCSGSSSNIGSNTVNWYQ |
| NO: 101 | AI7 LC (light chain) | QLPGTAPKLLIYSENTRPSGVPDRFSGSKSGTSASLAI |
| | | SGLQSEDEADYYCAAWDLNPAIGYVFGGGTKLTVLGQP |
| | | KAAPSVTLFPPSSEELQANKATLVCLISDFYPGAVTVA |
| | | WKADSSPVKAGVETTTPSKQSNNKYAASSYLSLTPEQW |
| | | KSHRSYSCQVTHEGSTVEKTVAPTECS |
| SEQ ID | CD28 binding part | DIVMTQTPLSLSVTPGQPASISCKSSDDLLRFSGKTYL |
| NO: 102 | AI8 LC (light chain) | YWYLQKPGQPPQLLIYEVSNRFSGVPDRFSGSGSGTDF |
| | | TLKISRVEAEDVGVYYCMQASRSKVAFTFGQGTKVEIK |
| | | RTVAAPSVFIFPPSDEQLKSGTASVVCLLNNFYPREAK |
| | | VQWKVDNALQSGNSQESVTEQDSKDSTYSLSSTLTLSK |
| | | ADYEKHKVYACEVTHQGLSSPVTKSFNRGEC |
| SEQ ID | CD28 binding part | DIVMTQTPLSLSVTPGQPASISCKSSGDLLSFDGKTYL |
| NO: 103 | AI9 LC (light chain) | YWYLQKPGQPPQLLIYEVSNRFSGVPDRFSGSGSGTDF |
| | | TLKISRVEAEDVGVYYCMQASKHRFGFTFGQGTKVEIK |
| | | RTVAAPSVFIFPPSDEQLKSGTASVVCLLNNFYPREAK |
| | | VQWKVDNALQSGNSQESVTEQDSKDSTYSLSSTLTLSK |
| | | ADYEKHKVYACEVTHQGLSSPVTKSFNRGEC |
| SEQ ID | CD28 binding part | DIVMTQTPLSLSVTPGQPASISCKSSGDLLEFAGKTYL |
| NO: 104 | AI10 LC (light chain) | YWYLQKPGQPPQLLIYEVSNRFSGVPDRFSGSGSGTDF |
| | | TLKISRVEAEDVGVYYCMQAHGSKIGFTFGQGTKVEIK |
| | | RTVAAPSVFIFPPSDEQLKSGTASVVCLLNNFYPREAK |
| | | VQWKVDNALQSGNSQESVTEQDSKDSTYSLSSTLTLSK |
| | | ADYEKHKVYACEVTHQGLSSPVTKSFNRGEC |
| SEQ ID | CD28 binding part | DIQMTQSPSTLSASVGDRVTITCRASQEIHSHLSWYQQ |
| NO: 105 | AI11 LC (light chain) | KPGKAPKLLIYSTSTLESGVPSRFSGSGSGTEFTLTIS |
| | | SLQPDDFATYYCQQGFRFAFSTFGQGTKVEIKRTVAAP |
| | | SVFIFPPSDEQLKSGTASVVCLLNNFYPREAKVQWKVD |
| | | NALQSGNSQESVTEQDSKDSTYSLSSTLTLSKADYEKH |
| | | KVYACEVTHQGLSSPVTKSFNRGEC |
| SEQ ID | CD28 binding part | DIVMTQSPDSLGVSLGERATINCKSSQSILYSSSNRNY |
| NO: 106 | AI12 LC (light chain) | LAWYQQKPGQPPKLLIYWASTRESGVPDRFSGSGSGTD |
| | | FTLTISSLQAEDVGVYYCQQNFRPPETFGGGTKVEIKR |
| | | TVAAPSVFIFPPSDEQLKSGTASVVCLLNNFYPREAKV |
| | | QWKVDNALQSGNSQESVTEQDSKDSTYSLSSTLTLSKA |
| | | DYEKHKVYACEVTHQGLSSPVTKSFNRGEC |
| SEQ ID | CD28 binding part | DIVMTQSPDSLAVSLGERATINCKSSQSVLYSSNNKNY |
| NO: 107 | AI13 LC (light chain) | LAWYQQKPGQPPKLLIYWASTRESGVPDRFSGSGSGTD |
| | | FTLTISSLQAEDVGVYYCQQNFRPPETFGGGTKVEIKR |
| | | TVAAPSVFIFPPSDEQLKSGTASVVCLLNNFYPREAKV |
| | | QWKVDNALQSGNSQESVTEQDSKDSTYSLSSTLTLSKA |
| | | DYEKHKVYACEVTHQGLSSPVTKSFNRGEC |
| SEQ ID | CD28 binding part | EIVLTQSPGTLSLSPGERATLSCRASQAVFSLDLHWYQ |
| NO: 108 | AI14 LC (light chain) | QKPGQAPRLLIYSTSKRATGIPDRFSGSGSGTDFTLTI |
| | | SRLEPEDFAVYYCQQGPAFKPDVTFGQGTKVEIKRTVA |
| | | APSVFIFPPSDEQLKSGTASVVCLLNNFYPREAKVQWK |
| | | VDNALQSGNSQESVTEQDSKDSTYSLSSTLTLSKADYE |
| | | KHKVYACEVTHQGLSSPVTKSENRGEC |
| SEQ ID | CD28 binding part | EIVLTQSPATLSLSPGERATLSCRASQSVSSYLAWYQQ |
| NO: 109 | AI15 LC (light chain) | KPGQAPRLLIYDASNRATGIPARFSGSGSGTDFTLTIS |
| | | SLEPEDFAVYYCHQRYRPPETFGQGTKVEIKRTVAAPS |
| | | VFIFPPSDEQLKSGTASVVCLLNNFYPREAKVQWKVDN |
| | | ALQSGNSQESVTEQDSKDSTYSLSSTLTLSKADYEKHK |
| | | VYACEVTHQGLSSPVTKSFNRGEC |
| SEQ ID | CD28 binding part | DIQMTQSPSSLSASVGDRVTITCRASQSIGAYLNWYQQ |
| NO: 110 | AI16 LC (light chain) | KPGKAPKLLIYHASDLQSGVPSRFSGSGSGTDFTLTIS |
| | | SLQPEDFATYYCQQYKDRPLTFGQGTKVEIKRTVAAPS |
| | | VFIFPPSDEQLKSGTASVVCLLNNFYPREAKVQWKVDN |
| | | ALQSGNSQESVTEQDSKDSTYSLSSTLTLSKADYEKHK |
| | | VYACEVTHQGLSSPVTKSFNRGEC |
| SEQ ID | CD28 binding part | DIVMTQTPLSLSVTPGQPASISCKSSGNLLTHDGKTYL |
| NO: 111 | AI17 LC (light chain) | YWYLQKPGQPPQLLIYEVSNRFSGVPDRFSGSGSGTDF |
| | | TLKISRVEAEDVGVYYCMQANALKFGFTFGQGTKVEIK |
| | | RTVAAPSVFIFPPSDEQLKSGTASVVCLLNNFYPREAK |
| | | VQWKVDNALQSGNSQESVTEQDSKDSTYSLSSTLTLSK |
| | | ADYEKHKVYACEVTHQGLSSPVTKSFNRGEC |
| SEQ ID | CD28 binding part | DIVMTQTPLSLSVTPGQPASISCKSSSDLLRFEGKTYL |
| NO: 112 | AI18 LC (light chain) | YWYLQKPGQPPQLLIYEVSNRFSGVPDRFSGSGSGTDF |
| | | TLKISRVEAEDVGVYYCMQAHASALKFTFGQGTKVEIK |
| | | RTVAAPSVFIFPPSDEQLKSGTASVVCLLNNFYPREAK |
| | | VQWKVDNALQSGNSQESVTEQDSKDSTYSLSSTLTLSK |
| | | ADYEKHKVYACEVTHQGLSSPVTKSFNRGEC |
| SEQ ID | CD28 binding part | DIVMTQTPLSLSVTPGQPASISCKSSGNLLTHGGKTYL |
| NO: 113 | AI19 LC (light chain) | YWYLQKPGQPPQLLIYEVSNRFSGVPDRFSGSGSGTDF |
| | | TLKISRVEAEDVGVYYCMQAHADGFGFTFGQGTKVEIK |
| | | RTVAAPSVFIFPPSDEQLKSGTASVVCLLNNFYPREAK |
| | | VQWKVDNALQSGNSQESVTEQDSKDSTYSLSSTLTLSK |
| | | ADYEKHKVYACEVTHQGLSSPVTKSFNRGEC |
| SEQ ID | CD28 binding part | DIVMTQTPLSLSVTPGQPASISCKSSGDLLRFDGKTYL |
| NO: 114 | AI20 LC (light chain) | YWYLQKPGQPPQLLIYEVSNRFSGVPDRFSGSGSGTDF |
| | | TLKISRVEAEDVGVYYCMQASETAVRFTFGQGTKVEIK |
| | | RTVAAPSVFIFPPSDEQLKSGTASVVCLLNNFYPREAK |
| | | VQWKVDNALQSGNSQESVTEQDSKDSTYSLSSTLTLSK |
| | | ADYEKHKVYACEVTHQGLSSPVTKSFNRGEC |
| SEQ ID | CD28 binding part | DIVMTQTPLSLSVTPGQPASISCKSSTDLLTFEGKTYL |
| NO: 115 | AI21 LC (light chain) | YWYLQKPGQPPQLLIYEVSNRFSGVPDRFSGSGSGTDF |
| | | TLKISRVEAEDVGVYYCMQAHSSKFKFTFGQGTKVEIK |
| | | RTVAAPSVFIFPPSDEQLKSGTASVVCLLNNFYPREAK |
| | | VQWKVDNALQSGNSQESVTEQDSKDSTYSLSSTLTLSK |
| | | ADYEKHKVYACEVTHQGLSSPVTKSFNRGEC |
| SEQ ID | CD28 binding part | DIVMTQTPLSLSVTPGQPASISCKSSGDLLSTDGKTYL |
| NO: 116 | AI22 LC (light chain) | YWYLQKPGQPPQLLIYEVSNRFSGVPDRFSGSGSGTDF |
| | | TLKISRVEAEDVGVYYCMQASGSRFYFTFGQGTKVEIK |
| | | RTVAAPSVFIFPPSDEQLKSGTASVVCLLNNFYPREAK |
| | | VQWKVDNALQSGNSQESVTEQDSKDSTYSLSSTLTLSK |
| | | ADYEKHKVYACEVTHQGLSSPVTKSFNRGEC |
| SEQ ID | CD28 binding part | DIVMTQSPDSLAVSLGERAIINCKSSQTLLYKYDNKNY |
| NO: 117 | AI23 LC (light chain) | LAWYQHKPGQPPKLLFYWASTRESGVPDRFSGSGSGTD |
| | | FTLTINSLQAEDVAVYYCHQYLYPAETFGQGTKLEIKR |
| | | TVAAPSVFIFPPSDEQLKSGTASVVCLLNNFYPREAKV |
| | | QWKVDNALQSGNSQESVTEQDSKDSTYSLSSTLTLSKA |
| | | DYEKHKVYACEVTHQGLSSPVTKSFNRGEC |
| SEQ ID | CEAxCD3 common | EVQLVESGGGLVQPGGSLKLSCAASGFTFNTYAMNWVR |
| NO: 118 | heavy chain | QAPGKGLEWVGRIRSKYNNYATYYADSVKDRFTISRDD |
| | | SKNTAYLQMNSLKTEDTAVYYCVRHGNFGNSYVSWFAY |
| | | WGQGTLVTVSSASTKGPSVFPLAPSSKSTSGGTAALGC |
| | | LVKDYFPEPVTVSWNSGALTSGVHTFPAVLQSSGLYSL |
| | | SSVVTVPSSSLGTQTYICNVNHKPSNTKVDKRVEPKSC |
| | | DKTHTCPPCPAPEAAGGPSVFLFPPKPKDTLMISRTPE |
| | | VTCVVVDVSHEDPEVKFNWYVDGVEVHNAKTKPREEQY |
| | | NSTYRVVSVLTVLHQDWLNGKEYKCKVSNKALAAPIEK |
| | | TISKAKGQPREPQVYTLPPSREEMTKNQVSLTCLVKGF |
| | | YPSDIAVEWESNGQPENNYKTTPPVLDSDGSFFLYSKL |
| | | TVDKSRWQQGNVFSCSVMHEALHNHYTQKSLSLSPG |
| SEQ ID | CEAxCD3 kappa | EIVLTQSPATLSLSPGERATLSCRASQSVNSNLNWYQQ |
| NO: 119 | light chain | KPGQAPRLLIYHSNNRPHGIPARFSGSGSGTDFTLTIS |
| | | SLEPEDFAVYYCQQFDYFREYNTFGQGTKVEIKRTVAA |
| | | PSVFIFPPSDEQLKSGTASVVCLLNNFYPREAKVQWKV |
| | | DNALQSGNSQESVTEQDSKDSTYSLSSTLTLSKADYEK |
| | | HKVYACEVTHQGLSSPVTKSFNRGEC |
| SEQ ID | CEAxCD3 lambda | QTVVTQEPSLTVSPGGTVTLTCRSSTGAVTTSNYANWF |
| NO: 120 | light chain | QQKPGQAPRGLIGGTNKRAPGTPARFSGSLLGGKAALT |
| | | LSGAQPEDEAEYYCALWYKQRWVFGGGTKLTVLGQPKA |
| | | APSVTLFPPSSEELQANKATLVCLISDFYPGAVTVAWK |
| | | ADSSPVKAGVETTTPSKQSNNKYAASSYLSLTPEQWKS |
| | | HRSYSCQVTHEGSTVEKTVAPTECS |
| SEQ ID | Y4 kappa light chain | DIQMTQSPSSLSASVGDRVTITCRASQSISSYLNWYQQ |
| NO: 121 | | KPGKAPKLLIYAASSLQSGVPSRFSGSGSGTDFTLTIS |
| | | SLQPEDFATYYCQQSYSTPNTFGQGTKVEIKRTVAAPS |
| | | VFIFPPSDEQLKSGTASVVCLLNNFYPREAKVQWKVDN |
| | | ALQSGNSQESVTEQDSKDSTYSLSSTLTLSKADYEKHK |
| | | VYACEVTHQGLSSPVTKSFNRGEC |
| SEQ ID | 1a28/AC84/N heavy | QSVLTQPPSVSAAPGQKVTISCSGSSSNIGIGLVSWYQ |
| NO: 122 | chain 1 (knob) VL | QLPGTAPKLLIYNVNTRPSGIPDRFSGSKSGTSATLGI |
| | CrossMab | TGLQTGDEADYYCGTWDFSYRVDFGGGTKLTVLSSAST |
| | | KGPSVFPLAPSSKSTSGGTAALGCLVKDYFPEPVTVSW |
| | | NSGALTSGVHTFPAVLQSSGLYSLSSVVTVPSSSLGTQ |
| | | TYICNVNHKPSNTKVDKKVEPKSCDKTHTCPPCPAPEA |
| | | AGGPSVFLFPPKPKDTLMISRTPEVTCVVVDVSHEDPE |
| | | VKFNWYVDGVEVHNAKTKPREEQYNSTYRVVSVLTVLH |
| | | QDWLNGKEYKCKVSNKALAAPIEKTISKAKGQPREPQV |
| | | YTLPPCRDELTKNQVSLWCLVKGFYPSDIAVEWQSNGQ |
| | | TENNYKTTPPVLDSDGSFFLYSKLTVDKSRWQQGNVFS |
| | | CSVMHEALHNHYTQKSLSLSPG |
| SEQ ID | 1a28/AC84/N heavy | QVQLQESGPGLVKPSETLSLTCTVSGGSISSYYWSWIR |
| NO: 123 | chain 2 (hole) | QPPGKGLEWIGYIYYSGITHYNPSLKSRVTISVDTSKI |
| | | QFSLKLSSVTAADTAVYYCARWGVRRDYYYYGMDVWGQ |
| | | GTTVTVSSASTKGPSVFPLAPSSKSTSGGTAALGCLVE |
| | | DYFPEPVTVTWNSGALSSGVHTFPAVLQSSGLYSLSSV |
| | | VTVPSSSLGTQTYICNVNHKPSNTKVDEKVEPKSCDKT |
| | | HTCPPCPAPEAAGGPSVFLFPPKPKDTLMISRTPEVTC |
| | | VVVDVSHEDPEVKFNWYVDGVEVHNAKTKPREEQYNST |
| | | YRVVSVLTVLHQDWLNGKEYKCKVSNKALAAPIEKTIS |
| | | KAKGQPREPQVCTLPPSRDELTKNQVSLSCAVKGFYPS |
| | | DIAVEWESNGQPENNYKTTPPVLDSDGSFFLVSKLTVD |
| | | KSRWQQGNVFSCSVMHEALHNHYTQKSLSLSPG |
| SEQ ID | 1a28/AC84/N light | EIVLTQSPGTLSLSPGERATLSCRASQSVSSSYLAWYQ |
| NO: 124 | chain 1 | QKPGQAPRLLIYGASSRATGIPDRFSGSGSGTDFTLTI |
| | | SRLEPEDFAVYYCQQYGSSPWTFGQGTKVEIKRTVAAP |
| | | SVFIFPPSDRKLKSGTASVVCLLNNFYPREAKVQWKVD |
| | | NALQSGNSQESVTEQDSKDSTYSLSSTLTLSKADYEKH |
| | | KVYACEVTHQGLSSPVTKSFNRGEC |
| SEQ ID | la28/AC84/N light | EVQLLESGGGLVQPGGSLRLSCAASGFTFSSYAMSWVR |
| NO: 125 | chain 2 VHd | QAPGKGLEWVSAISGSGGSTYYADSVKGRFTISRDNSK |
| | CrossMab | NTLYLQMNSLRAEDTAVYYCAKSYGAFDYWGQGTLVTV |
| | | SSASVAAPSVFIFPPSDEQLKSGTASVVCLLNNFYPRE |
| | | AKVQWKVDNALQSGNSQESVTEQDSKDSTYSLSSTLTL |
| | | SKADYEKHKVYACEVTHQGLSSPVTKSFNRGEC |
| SEQ ID | TGN1412 heavy | QVQLVQSGAEVKKPGASVKVSCKASGYTFTSYYIHWVR |
| NO: 126 | chain | QAPGQGLEWIGCIYPGNVNTNYNEKFKDRATLTVDTSI |
| | | STAYMELSRLRSDDTAVYFCTRSHYGLDWNFDVWGQGT |
| | | TVTVSSASTKGPSVFPLAPCSRSTSESTAALGCLVKDY |
| | | FPEPVTVSWNSGALTSGVHTFPAVLQSSGLYSLSSVVT |
| | | VPSSSLGTKTYTCNVDHKPSNTKVDKRVESKYGPPCPS |
| | | CPAPEFLGGPSVFLFPPKPKDTLMISRTPEVTCVVVDV |
| | | SQEDPEVQFNWYVDGVEVHNAKTKPREEQFNSTYRVVS |
| | | VLTVLHQDWLNGKEYKCKVSNKGLPSSIEKTISKAKGQ |
| | | PREPQVYTLPPSQEEMTKNQVSLTCLVKGFYPSDIAVE |
| | | WESNGQPENNYKTTPPVLDSDGSFFLYSRLTVDKSRWQ |
| | | EGNVFSCSVMHEALHNHYTQKSLSLSLGK |
| SEQ ID | TGN1412 light chain | DIQMTQSPSSLSASVGDRVTITCHASQNIYVWLNWYQQ |
| NO: 127 | | KPGKAPKLLIYKASNLHTGVPSRFSGSGSGTDFTLTIS |
| | | SLQPEDFATYYCQQGQTYPYTFGGGTKVEIKRTVAAPS |
| | | VFIFPPSDEQLKSGTASVVCLLNNFYPREAKVQWKVDN |
| | | ALQSGNSQESVTEQDSKDSTYSLSSTLTLSKADYEKHK |
| | | VYACEVTHQGLSSPVTKSFNRGEC |
| SEQ ID | MSLN binding part | SSNIAHGP |
| NO: 128 | O30 CDR-L1 | |
| SEQ ID | MSLN binding part | ATN |
| NO: 129 | O30 CDR-L2 | |
| SEQ ID | MSLN binding part | AAYDLTGWFAYAV |
| NO: 130 | O30 CDR-L3 | |
| SEQ ID | MSLN binding part | SGISVKDYR |
| NO: 131 | O25 CDR-L1 | |
| SEQ ID | MSLN binding part | YKSNSDM |
| NO: 132 | O25 CDR-L2 | |
| SEQ ID | MSLN binding part | MIWHHGHGTSLV |
| NO: 133 | O25 CDR-L3 | |
| SEQ ID | MSLN binding part | SDIRVRDYR |
| NO: 134 | O35 CDR-L1 | |
| SEQ ID | MSLN binding part | YKTDSDK |
| NO: 135 | O35 CDR-L2 | |
| SEQ ID | MSLN binding part | MIWHRTTGTSLV |
| NO: 136 | O35 CDR-L3 | |
| SEQ ID | MSLN binding part | SGINVRDYR |
| NO: 137 | O38 CDR-L1 | |
| SEQ ID | MSLN binding part | YKSASDK |
| NO: 138 | O38 CDR-L2 | |
| SEQ ID | MSLN binding part | MIWHHDSEGHAFV |
| NO: 139 | O38 CDR-L3 | |
| SEQ ID | MSLN binding part | KIGHRA |
| NO: 140 | O41 CDR-L1 | |
| SEQ ID | MSLN binding part | YTY |
| NO: 141 | O41 CDR-L2 | |
| SEQ ID | MSLN binding part | QVWDWYSEGGVV |
| NO: 142 | O41 CDR-L3 | |
| SEQ ID | MSLN binding part | QSVLTQPPSASGTPGQRVTISCSGSSSNIAHGPVNWYQ |
| NO: 143 | O30 VL (light chain | QLPGTAPKLLIYATNHRPSGVPDRFSGSKSGTTASLTI |
| | variable region) | SGLQSEDEADYYCAAYDLTGWFAYAVFGGGTKLTVLGQ |
| | | PKAAPSVTL |
| SEQ ID | MSLN binding part | QPVLTQPASLSASPGASASLTCTLHSGISVKDYRIYWY |
| NO: 144 | O25 VL (light chain | QQKPGRPPQYLLRYKSNSDMQQGSGVPSRFSGSKDASA |
| | variable region) | NAGILLISGLQSEDEADYYCMIWHHGHGTSLVFGGGTK |
| | | LTVL |
| SEQ ID | MSLN binding part | QPVLTQPVSLSASPGASVSLTCTLRSDIRVRDYRIFWY |
| NO: 145 | O35 VL (light chain | QQKPGSPPQYLLRYKTDSDKQQGSGVPSRFSGSKDASA |
| | variable region) | NAGILLISGLQSEDEADYYCMIWHRTTGTSLVFGGGTK |
| | | LTVL |
| SEQ ID | MSLN binding part | QPVLTQPASLSASPGASASLTCTLRSGINVRDYRIFWY |
| NO: 146 | O38 VL (light chain | QQKPGSPPQYLLRYKSASDKQQGSGVPSRFSGSKDASA |
| | variable region) | NAGILLISGLQSEDEADYYCMIWHHDSEGHAFVFGGGT |
| | | KLTVL |
| SEQ ID | MSLN binding part | SYVLTQPPSVSVAPGKTARITCGGNKIGHRAVHWYQQK |
| NO: 147 | O41 VL (light chain | PGQAPVLVIYYTYERPSGIPERFSGSNSGNTATLTISR |
| | variable region) | VEAGDEADYYCQVWDWYSEGGVVFGGGTKLTVLGQPKA |
| | | APSVTL |
| SEQ ID | MSLN binding part | QSVLTQPPSASGTPGQRVTISCSGSSSNIAHGPVNWYQ |
| NO: 148 | O30 LC (light chain) | QLPGTAPKLLIYATNHRPSGVPDRFSGSKSGTTASLTI |
| | | SGLQSEDEADYYCAAYDLTGWFAYAVFGGGTKLTVLGQ |
| | | PKAAPSVTLFPPSSEELQANKATLVCLISDFYPGAVTV |
| | | AWKADSSPVKAGVETTTPSKQSNNKYAASSYLSLTPEQ |
| | | WKSHRSYSCQVTHEGSTVEKTVAPTECS |
| SEQ ID | MSLN binding part | QPVLTQPASLSASPGASASLTCTLHSGISVKDYRIYWY |
| NO: 149 | O25 LC (light chain) | QQKPGRPPQYLLRYKSNSDMQQGSGVPSRFSGSKDASA |
| | | NAGILLISGLQSEDEADYYCMIWHHGHGTSLVFGGGTK |
| | | LTVLGQPKAAPSVTLFPPSSEELQANKATLVCLISDFY |
| | | PGAVTVAWKADSSPVKAGVETTTPSKQSNNKYAASSYL |
| | | SLTPEQWKSHRSYSCQVTHEGSTVEKTVAPTECS |
| SEQ ID | MSLN binding part | QPVLTQPVSLSASPGASVSLTCTLRSDIRVRDYRIFWY |
| NO: 150 | O35 LC (light chain) | QQKPGSPPQYLLRYKTDSDKQQGSGVPSRFSGSKDASA |
| | | NAGILLISGLQSEDEADYYCMIWHRTTGTSLVFGGGTK |
| | | LTVLGQPKAAPSVTLFPPSSEELQANKATLVCLISDFY |
| | | PGAVTVAWKADSSPVKAGVETTTPSKQSNNKYAASSYL |
| | | SLTPEQWKSHRSYSCQVTHEGSTVEKTVAPTECS |
| SEQ ID | MSLN binding part | QPVLTQPASLSASPGASASLTCTLRSGINVRDYRIFWY |
| NO: 151 | O38 LC (light chain) | QQKPGSPPQYLLRYKSASDKQQGSGVPSRFSGSKDASA |
| | | NAGILLISGLQSEDEADYYCMIWHHDSEGHAFVFGGGT |
| | | KLTVLGQPKAAPSVTLFPPSSEELQANKATLVCLISDF |
| | | YPGAVTVAWKADSSPVKAGVETTTPSKQSNNKYAASSY |
| | | LSLTPEQWKSHRSYSCQVTHEGSTVEKTVAPTECS |
| SEQ ID | MSLN binding part | SYVLTQPPSVSVAPGKTARITCGGNKIGHRAVHWYQQK |
| NO: 152 | O41 LC (light chain) | PGQAPVLVIYYTYERPSGIPERFSGSNSGNTATLTISR |
| | | VEAGDEADYYCQVWDWYSEGGVVFGGGTKLTVLGQPKA |
| | | APSVTLFPPSSEELQANKATLVCLISDFYPGAVTVAWK |
| | | ADSSPVKAGVETTTPSKQSNNKYAASSYLSLTPEQWKS |
| | | HRSYSCQVTHEGSTVEKTVAPTECS |

Table 2 shows exemplary bispecific antibodies of the disclosure. Nomenclature for each antibodies are shown and the individual kappa light chain, lambda light chain and heavy chain regions of the κλ-Bodies of the disclosure are described. “AI” denotes anti-Human CD28 antigen binding regions, “AC” denotes Anti-human CEA antigen binding regions, “0” denotes anti-Human MSLN antigen binding regions. /N denotes a heavy chain with LALAPA mutation. Alternate nomenclature is shown within the brackets.

TABLE 2
Exemplary Bispecific Antibodies of the Disclosure

| | | Lambda light | |
|---|---|---|---|
| BsAb name | Kappa light chain | chain | Heavy chain |
| AI3AC84/N | AI3 (AI_1H5) | AC84 | Common VH |
| AI5AC84/N | AI5 (AI_1A9) | (AC1g6_1F1) | with LALAPA |
| AI8AC84/N | AI8 (AI_2A12) | | mutation |
| AI9AC84/N | AI9 (AI_1A7) | | |
| AI10AC84/N | AI10 (AI_1B6) | | |
| AI11AC84/N | AI11 (AI_1E11) | | |
| AI12AC84/N | AI12 (AI_2E2) | | |
| AI13AC84/N | AI13 (AI_2H10) | | |
| AI14AC84/N | AI14 (AI_1B8) | | |
| AI15AC84/N | AI15 (AI_2E3) | | |
| AI16AC84/N | AI16 (AI_1G3) | | |
| AI17AC84/N | AI17 (AI_2C10) | | |
| AI18AC84/N | AI18 (AI_2G3) | | |
| AI19AC84/N | AI19 (AI_1G4) | | |
| AI20AC84/N | AI20 (AI_1A1) | | |
| AI21AC84/N | AI21 (AI_1F2) | | |
| AI22AC84/N | AI22 (AI_1F8) | | |
| AI23AC84/N | AI23 (AI_1D4) | | |
| AC61AI7/N | AC61 (AC1b7_1B9) | AI7 (AI_1A12) | |
| AI3O30/N | AI3 (AI_1H5) | O30 (O1b6_1D1) | |
| AI3O25/N | AI3 (AI_1H5) | O25 (Ob8_1E1) | |
| AI3O35/N | AI3 (AI_1H5) | O35 (Ob8_A10) | |
| AI3O38/N | AI3 (AI_1H5) | O38 (Oh1_A4) | |
| AI3O41/N | AI3 (AI_1H5) | O41 (Ob9_1A11) | |
| AI5O30/N | AI5 (AI_1A9) | O30 (O1b6_1D1) | |
| AI5O25/N | AI5 (AI_1A9) | O25 (Ob8_1E1) | |
| AI5O35/N | AI5 (AI_1A9) | O35 (Ob8_A10) | |
| AI5O38/N | AI5 (AI_1A9) | O38 (Oh1_A4) | |
| AI5O41/N | AI5 (AI_1A9) | O41 (Ob9_1A11) | |
| AI10O30/N | AI10 (AI_1B6) | O30 (O1b6_1D1) | |
| AI10O25/N | AI10 (AI_1B6) | O25 (Ob8_1E1) | |
| AI10O35/N | AI10 (AI_1B6) | O35 (Ob8_A10) | |
| AI10O38/N | AI10 (AI_1B6) | O38 (Oh1_A4) | |
| AI10O41/N | AI10 (AI_1B6) | O41 (Ob9_1A11) | |
| AI13O30/N | AI13 (AI_2H10) | O30 (O1b6_1D1) | |
| AI13O25/N | AI13 (AI_2H10) | O25 (Ob8_1E1) | |
| AI13O35/N | AI13 (AI_2H10) | O35 (Ob8_A10) | |
| AI13O38/N | AI13 (AI_2H10) | O38 (Oh1_A4) | |
| AI13O41/N | AI13 (AI_2H10) | O41 (Ob9_1A11) | |

In some embodiments, the AI3AC84/N bispecific antibody has a heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ TD NO: 3, a kappa light chain comprising a CDRL1 comprising the amino acid sequence of SEQ ID NO: 23, a CDRL2 comprising the amino acid sequence of SEQ TD NO: 24, and a CDRL3 comprising the amino acid sequence of SEQ ID NO: 25, and a lambda light chain comprising a CDRL1 comprising the amino acid sequence of SEQ ID NO: 14, a CDRL2 comprising the amino acid sequence of SEQ ID NO: 15, and a CDRL3 comprising the amino acid sequence of SEQ ID NO: 16.

In some embodiments, the AI3AC84/N bispecific antibody has a heavy chain variable region comprising the amino acid sequence of SEQ ID NO: 4, a kappa light chain variable region comprising the amino acid sequence of SEQ ID NO: 80, and a lambda light chain variable region comprising the amino acid sequence of SEQ ID NO: 19.

In some embodiments, the AI3AC84/N bispecific antibody has a heavy chain variable and constant region comprising the amino acid sequence of SEQ ID NO: 7, a kappa light chain comprising the amino acid sequence of SEQ ID NO: 99, and a lambda light chain comprising the amino acid sequence of SEQ ID NO: 22.

In some embodiments, the AI5AC84/N bispecific antibody has a heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 26, a CDRL2 comprising the amino acid sequence of SEQ ID NO: 27, and a CDRL3 comprising the amino acid sequence of SEQ ID NO: 28, and a lambda light chain comprising a CDRL1 comprising the amino acid sequence of SEQ ID NO: 14, a CDRL2 comprising the amino acid sequence of SEQ ID NO: 15, and a CDRL3 comprising the amino acid sequence of SEQ ID NO: 16.

In some embodiments, the AI5AC84/N bispecific antibody has a heavy chain variable region comprising the amino acid sequence of SEQ ID NO: 4, a kappa light chain variable region comprising the amino acid sequence of SEQ ID NO: 81, and a lambda light chain variable region comprising the amino acid sequence of SEQ ID NO: 19.

In some embodiments, the AI5AC84/N bispecific antibody has a heavy chain variable and constant region comprising the amino acid sequence of SEQ ID NO: 7, a kappa light chain comprising the amino acid sequence of SEQ ID NO: 100, and a lambda light chain comprising the amino acid sequence of SEQ ID NO: 22.

In some embodiments, the AI8AC84/N bispecific antibody has a heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 32, a CDRL2 comprising the amino acid sequence of SEQ ID NO: 33, and a CDRL3 comprising the amino acid sequence of SEQ ID NO: 34, and a lambda light chain comprising a CDRL1 comprising the amino acid sequence of SEQ ID NO: 14, a CDRL2 comprising the amino acid sequence of SEQ ID NO: 15, and a CDRL3 comprising the amino acid sequence of SEQ ID NO: 16.

In some embodiments, the AI8AC84/N bispecific antibody has a heavy chain variable region comprising the amino acid sequence of SEQ ID NO: 4, a kappa light chain variable region comprising the amino acid sequence of SEQ ID NO: 83, and a lambda light chain variable region comprising the amino acid sequence of SEQ ID NO: 19.

In some embodiments, the AI8AC84/N bispecific antibody has a heavy chain variable and constant region comprising the amino acid sequence of SEQ ID NO: 7, a kappa light chain comprising the amino acid sequence of SEQ ID NO: 102, and a lambda light chain comprising the amino acid sequence of SEQ ID NO: 22.

In some embodiments, the AI9AC84/N bispecific antibody has a heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 35, a CDRL2 comprising the amino acid sequence of SEQ ID NO: 36, and a CDRL3 comprising the amino acid sequence of SEQ ID NO: 37, and a lambda light chain comprising a CDRL1 comprising the amino acid sequence of SEQ ID NO: 14, a CDRL2 comprising the amino acid sequence of SEQ ID NO: 15, and a CDRL3 comprising the amino acid sequence of SEQ ID NO: 16.

In some embodiments, the AI9AC84/N bispecific antibody has a heavy chain variable region comprising the amino acid sequence of SEQ ID NO: 4, a kappa light chain variable region comprising the amino acid sequence of SEQ ID NO: 84, and a lambda light chain variable region comprising the amino acid sequence of SEQ ID NO: 19.

In some embodiments, the AI9AC84/N bispecific antibody has a heavy chain variable and constant region comprising the amino acid sequence of SEQ ID NO: 7, a kappa light chain comprising the amino acid sequence of SEQ ID NO: 103, and a lambda light chain comprising the amino acid sequence of SEQ ID NO: 22.

In some embodiments, the AI10AC84/N bispecific antibody has a heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 38, a CDRL2 comprising the amino acid sequence of SEQ ID NO: 39, and a CDRL3 comprising the amino acid sequence of SEQ ID NO: 40, and a lambda light chain comprising a CDRL1 comprising the amino acid sequence of SEQ ID NO: 14, a CDRL2 comprising the amino acid sequence of SEQ ID NO: 15, and a CDRL3 comprising the amino acid sequence of SEQ ID NO: 16.

In some embodiments, the AI10AC84/N bispecific antibody has a heavy chain variable region comprising the amino acid sequence of SEQ ID NO: 4, a kappa light chain variable region comprising the amino acid sequence of SEQ ID NO: 85, and a lambda light chain variable region comprising the amino acid sequence of SEQ ID NO: 19.

In some embodiments, the AI10AC84/N bispecific antibody has a heavy chain variable and constant region comprising the amino acid sequence of SEQ ID NO: 7, a kappa light chain comprising the amino acid sequence of SEQ ID NO: 104, and a lambda light chain comprising the amino acid sequence of SEQ ID NO: 22.

In some embodiments, the AI11AC84/N bispecific antibody has a heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 41, a CDRL2 comprising the amino acid sequence of SEQ ID NO: 42, and a CDRL3 comprising the amino acid sequence of SEQ ID NO: 43, and a lambda light chain comprising a CDRL1 comprising the amino acid sequence of SEQ ID NO: 14, a CDRL2 comprising the amino acid sequence of SEQ ID NO: 15, and a CDRL3 comprising the amino acid sequence of SEQ ID NO: 16.

In some embodiments, the AI11AC84/N bispecific antibody has a heavy chain variable region comprising the amino acid sequence of SEQ ID NO: 4, a kappa light chain variable region comprising the amino acid sequence of SEQ ID NO: 86, and a lambda light chain variable region comprising the amino acid sequence of SEQ ID NO: 19.

In some embodiments, the AI11AC84/N bispecific antibody has a heavy chain variable and constant region comprising the amino acid sequence of SEQ ID NO: 7, a kappa light chain comprising the amino acid sequence of SEQ ID NO: 105, and a lambda light chain comprising the amino acid sequence of SEQ ID NO: 22.

In some embodiments, the AI12AC84/N bispecific antibody has a heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 44, a CDRL2 comprising the amino acid sequence of SEQ ID NO: 45, and a CDRL3 comprising the amino acid sequence of SEQ ID NO: 46, and a lambda light chain comprising a CDRL1 comprising the amino acid sequence of SEQ ID NO: 14, a CDRL2 comprising the amino acid sequence of SEQ ID NO: 15, and a CDRL3 comprising the amino acid sequence of SEQ ID NO: 16.

In some embodiments, the AI12AC84/N bispecific antibody has a heavy chain variable region comprising the amino acid sequence of SEQ ID NO: 4, a kappa light chain variable region comprising the amino acid sequence of SEQ ID NO: 87, and a lambda light chain variable region comprising the amino acid sequence of SEQ ID NO: 19.

In some embodiments, the AI12AC84/N bispecific antibody has a heavy chain variable and constant region comprising the amino acid sequence of SEQ ID NO: 7, a kappa light chain comprising the amino acid sequence of SEQ ID NO: 106, and a lambda light chain comprising the amino acid sequence of SEQ ID NO: 22.

In some embodiments, the AI13AC84/N bispecific antibody has a heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 47, a CDRL2 comprising the amino acid sequence of SEQ ID NO: 48, and a CDRL3 comprising the amino acid sequence of SEQ ID NO: 49, and a lambda light chain comprising a CDRL1 comprising the amino acid sequence of SEQ ID NO: 14, a CDRL2 comprising the amino acid sequence of SEQ ID NO: 15, and a CDRL3 comprising the amino acid sequence of SEQ ID NO: 16.

In some embodiments, the AI13AC84/N bispecific antibody has a heavy chain variable region comprising the amino acid sequence of SEQ ID NO: 4, a kappa light chain variable region comprising the amino acid sequence of SEQ ID NO: 88, and a lambda light chain variable region comprising the amino acid sequence of SEQ ID NO: 19.

In some embodiments, the AI13AC84/N bispecific antibody has a heavy chain variable and constant region comprising the amino acid sequence of SEQ ID NO: 7, a kappa light chain comprising the amino acid sequence of SEQ ID NO: 107, and a lambda light chain comprising the amino acid sequence of SEQ ID NO: 22.

In some embodiments, the AI14AC84/N bispecific antibody has a heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 50, a CDRL2 comprising the amino acid sequence of SEQ ID NO: 51, and a CDRL3 comprising the amino acid sequence of SEQ ID NO: 52, and a lambda light chain comprising a CDRL1 comprising the amino acid sequence of SEQ ID NO: 14, a CDRL2 comprising the amino acid sequence of SEQ ID NO: 15, and a CDRL3 comprising the amino acid sequence of SEQ ID NO: 16.

In some embodiments, the AI14AC84/N bispecific antibody has a heavy chain variable region comprising the amino acid sequence of SEQ ID NO: 4, a kappa light chain variable region comprising the amino acid sequence of SEQ ID NO: 89, and a lambda light chain variable region comprising the amino acid sequence of SEQ ID NO: 19.

In some embodiments, the AI14AC84/N bispecific antibody has a heavy chain variable and constant region comprising the amino acid sequence of SEQ ID NO: 7, a kappa light chain comprising the amino acid sequence of SEQ ID NO: 108, and a lambda light chain comprising the amino acid sequence of SEQ ID NO: 22.

In some embodiments, the AI15AC84/N bispecific antibody has a heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 53, a CDRL2 comprising the amino acid sequence of SEQ ID NO: 54, and a CDRL3 comprising the amino acid sequence of SEQ ID NO: 55, and a lambda light chain comprising a CDRL1 comprising the amino acid sequence of SEQ ID NO: 14, a CDRL2 comprising the amino acid sequence of SEQ ID NO: 15, and a CDRL3 comprising the amino acid sequence of SEQ ID NO: 16.

In some embodiments, the AI15AC84/N bispecific antibody has a heavy chain variable region comprising the amino acid sequence of SEQ ID NO: 4, a kappa light chain variable region comprising the amino acid sequence of SEQ ID NO: 90, and a lambda light chain variable region comprising the amino acid sequence of SEQ ID NO: 19.

In some embodiments, the AI15AC84/N bispecific antibody has a heavy chain variable and constant region comprising the amino acid sequence of SEQ ID NO: 7, a kappa light chain comprising the amino acid sequence of SEQ ID NO: 109, and a lambda light chain comprising the amino acid sequence of SEQ ID NO: 22.

In some embodiments, the AI16AC84/N bispecific antibody has a heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 56, a CDRL2 comprising the amino acid sequence of SEQ ID NO: 57, and a CDRL3 comprising the amino acid sequence of SEQ ID NO: 58, and a lambda light chain comprising a CDRL1 comprising the amino acid sequence of SEQ ID NO: 14, a CDRL2 comprising the amino acid sequence of SEQ ID NO: 15, and a CDRL3 comprising the amino acid sequence of SEQ ID NO: 16.

In some embodiments, the AI16AC84/N bispecific antibody has a heavy chain variable region comprising the amino acid sequence of SEQ ID NO: 4, a kappa light chain variable region comprising the amino acid sequence of SEQ ID NO: 91, and a lambda light chain variable region comprising the amino acid sequence of SEQ ID NO: 19.

In some embodiments, the AI16AC84/N bispecific antibody has a heavy chain variable and constant region comprising the amino acid sequence of SEQ ID NO: 7, a kappa light chain comprising the amino acid sequence of SEQ ID NO: 110, and a lambda light chain comprising the amino acid sequence of SEQ ID NO: 22.

In some embodiments, the AI17AC84/N bispecific antibody has a heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 59, a CDRL2 comprising the amino acid sequence of SEQ ID NO: 60, and a CDRL3 comprising the amino acid sequence of SEQ ID NO: 61, and a lambda light chain comprising a CDRL1 comprising the amino acid sequence of SEQ ID NO: 14, a CDRL2 comprising the amino acid sequence of SEQ ID NO: 15, and a CDRL3 comprising the amino acid sequence of SEQ ID NO: 16.

In some embodiments, the AI17AC84/N bispecific antibody has a heavy chain variable region comprising the amino acid sequence of SEQ ID NO: 4, a kappa light chain variable region comprising the amino acid sequence of SEQ ID NO: 92, and a lambda light chain variable region comprising the amino acid sequence of SEQ ID NO: 19.

In some embodiments, the AI17AC84/N bispecific antibody has a heavy chain variable and constant region comprising the amino acid sequence of SEQ ID NO: 7, a kappa light chain comprising the amino acid sequence of SEQ ID NO: 111, and a lambda light chain comprising the amino acid sequence of SEQ ID NO: 22.

In some embodiments, the AI18AC84/N bispecific antibody has a heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 62, a CDRL2 comprising the amino acid sequence of SEQ ID NO: 63, and a CDRL3 comprising the amino acid sequence of SEQ ID NO: 64, and a lambda light chain comprising a CDRL1 comprising the amino acid sequence of SEQ ID NO: 14, a CDRL2 comprising the amino acid sequence of SEQ ID NO: 15, and a CDRL3 comprising the amino acid sequence of SEQ ID NO: 16.

In some embodiments, the AI18AC84/N bispecific antibody has a heavy chain variable region comprising the amino acid sequence of SEQ ID NO: 4, a kappa light chain variable region comprising the amino acid sequence of SEQ ID NO: 93, and a lambda light chain variable region comprising the amino acid sequence of SEQ ID NO: 19.

In some embodiments, the AI18AC84/N bispecific antibody has a heavy chain variable and constant region comprising the amino acid sequence of SEQ ID NO: 7, a kappa light chain comprising the amino acid sequence of SEQ ID NO: 112, and a lambda light chain comprising the amino acid sequence of SEQ ID NO: 22.

In some embodiments, the AI19AC84/N bispecific antibody has a heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 65, a CDRL2 comprising the amino acid sequence of SEQ ID NO: 66, and a CDRL3 comprising the amino acid sequence of SEQ ID NO: 67, and a lambda light chain comprising a CDRL1 comprising the amino acid sequence of SEQ ID NO: 14, a CDRL2 comprising the amino acid sequence of SEQ ID NO: 15, and a CDRL3 comprising the amino acid sequence of SEQ ID NO: 16.

In some embodiments, the AI19AC84/N bispecific antibody has a heavy chain variable region comprising the amino acid sequence of SEQ ID NO: 4, a kappa light chain variable region comprising the amino acid sequence of SEQ ID NO: 94, and a lambda light chain variable region comprising the amino acid sequence of SEQ ID NO: 19.

In some embodiments, the AI19AC84/N bispecific antibody has a heavy chain variable and constant region comprising the amino acid sequence of SEQ ID NO: 7, a kappa light chain comprising the amino acid sequence of SEQ ID NO: 113, and a lambda light chain comprising the amino acid sequence of SEQ ID NO: 22.

In some embodiments, the AI20AC84/N bispecific antibody has a heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 68, a CDRL2 comprising the amino acid sequence of SEQ ID NO: 69, and a CDRL3 comprising the amino acid sequence of SEQ ID NO: 70, and a lambda light chain comprising a CDRL1 comprising the amino acid sequence of SEQ ID NO: 14, a CDRL2 comprising the amino acid sequence of SEQ ID NO: 15, and a CDRL3 comprising the amino acid sequence of SEQ ID NO: 16.

In some embodiments, the AI20AC84/N bispecific antibody has a heavy chain variable region comprising the amino acid sequence of SEQ ID NO: 4, a kappa light chain variable region comprising the amino acid sequence of SEQ ID NO: 95, and a lambda light chain variable region comprising the amino acid sequence of SEQ ID NO: 19.

In some embodiments, the AI20AC84/N bispecific antibody has a heavy chain variable and constant region comprising the amino acid sequence of SEQ ID NO: 7, a kappa light chain comprising the amino acid sequence of SEQ ID NO: 114, and a lambda light chain comprising the amino acid sequence of SEQ ID NO: 22.

In some embodiments, the AI21AC84/N bispecific antibody has a heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 71, a CDRL2 comprising the amino acid sequence of SEQ ID NO: 72, and a CDRL3 comprising the amino acid sequence of SEQ ID NO: 73, and a lambda light chain comprising a CDRL1 comprising the amino acid sequence of SEQ ID NO: 14, a CDRL2 comprising the amino acid sequence of SEQ ID NO: 15, and a CDRL3 comprising the amino acid sequence of SEQ ID NO: 16.

In some embodiments, the AI21AC84/N bispecific antibody has a heavy chain variable region comprising the amino acid sequence of SEQ ID NO: 4, a kappa light chain variable region comprising the amino acid sequence of SEQ ID NO: 96, and a lambda light chain variable region comprising the amino acid sequence of SEQ ID NO: 19.

In some embodiments, the AI21AC84/N bispecific antibody has a heavy chain variable and constant region comprising the amino acid sequence of SEQ ID NO: 7, a kappa light chain comprising the amino acid sequence of SEQ ID NO: 115, and a lambda light chain comprising the amino acid sequence of SEQ ID NO: 22.

In some embodiments, the AI22AC84/N bispecific antibody has a heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 74, a CDRL2 comprising the amino acid sequence of SEQ ID NO: 75, and a CDRL3 comprising the amino acid sequence of SEQ ID NO: 76, and a lambda light chain comprising a CDRL1 comprising the amino acid sequence of SEQ ID NO: 14, a CDRL2 comprising the amino acid sequence of SEQ ID NO: 15, and a CDRL3 comprising the amino acid sequence of SEQ ID NO: 16.

In some embodiments, the AI22AC84/N bispecific antibody has a heavy chain variable region comprising the amino acid sequence of SEQ ID NO: 4, a kappa light chain variable region comprising the amino acid sequence of SEQ ID NO: 97, and a lambda light chain variable region comprising the amino acid sequence of SEQ ID NO: 19.

In some embodiments, the AI22AC84/N bispecific antibody has a heavy chain variable and constant region comprising the amino acid sequence of SEQ ID NO: 7, a kappa light chain comprising the amino acid sequence of SEQ ID NO: 116, and a lambda light chain comprising the amino acid sequence of SEQ ID NO: 22.

In some embodiments, the AI23AC84/N bispecific antibody has a heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 77, a CDRL2 comprising the amino acid sequence of SEQ ID NO: 78, and a CDRL3 comprising the amino acid sequence of SEQ ID NO: 79, and a lambda light chain comprising a CDRL1 comprising the amino acid sequence of SEQ ID NO: 14, a CDRL2 comprising the amino acid sequence of SEQ ID NO: 15, and a CDRL3 comprising the amino acid sequence of SEQ ID NO: 16.

In some embodiments, the AI23AC84/N bispecific antibody has a heavy chain variable region comprising the amino acid sequence of SEQ ID NO: 4, a kappa light chain variable region comprising the amino acid sequence of SEQ ID NO: 98, and a lambda light chain variable region comprising the amino acid sequence of SEQ ID NO: 19.

In some embodiments, the AI23AC84/N bispecific antibody has a heavy chain variable and constant region comprising the amino acid sequence of SEQ ID NO: 7, a kappa light chain comprising the amino acid sequence of SEQ ID NO: 117, and a lambda light chain comprising the amino acid sequence of SEQ ID NO: 22.

In some embodiments, the AC61AI7/N bispecific antibody has a heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 11, a CDRL2 comprising the amino acid sequence of SEQ ID NO: 12, and a CDRL3 comprising the amino acid sequence of SEQ ID NO: 13, and a lambda light chain comprising a CDRL1 comprising the amino acid sequence of SEQ ID NO: 29, a CDRL2 comprising the amino acid sequence of SEQ ID NO: 30, and a CDRL3 comprising the amino acid sequence of SEQ ID NO: 31.

In some embodiments, the AC61AI7/N bispecific antibody has a heavy chain variable region comprising the amino acid sequence of SEQ ID NO: 4, a kappa light chain variable region comprising the amino acid sequence of SEQ ID NO: 18, and a lambda light chain variable region comprising the amino acid sequence of SEQ ID NO: 82.

In some embodiments, the AC61AI7/N bispecific antibody has a heavy chain variable and constant region comprising the amino acid sequence of SEQ ID NO: 7, a kappa light chain comprising the amino acid sequence of SEQ ID NO: 21, and a lambda light chain comprising the amino acid sequence of SEQ ID NO: 101.

In some embodiments, the AI3O30/N bispecific antibody has a heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 23, a CDRL2 comprising the amino acid sequence of SEQ ID NO: 24, and a CDRL3 comprising the amino acid sequence of SEQ ID NO: 25, and a lambda light chain comprising a CDRL1 comprising the amino acid sequence of SEQ ID NO: 128, a CDRL2 comprising the amino acid sequence of SEQ ID NO: 129, and a CDRL3 comprising the amino acid sequence of SEQ ID NO: 130.

In some embodiments, the AI3O30/N bispecific antibody has a heavy chain variable region comprising the amino acid sequence of SEQ ID NO: 4, a kappa light chain variable region comprising the amino acid sequence of SEQ ID NO: 80, and a lambda light chain variable region comprising the amino acid sequence of SEQ ID NO: 143.

In some embodiments, the AI3O30/N bispecific antibody has a heavy chain variable and constant region comprising the amino acid sequence of SEQ ID NO: 7, a kappa light chain comprising the amino acid sequence of SEQ ID NO: 99, and a lambda light chain comprising the amino acid sequence of SEQ ID NO: 148.

In some embodiments, the AI3O25/N bispecific antibody has a heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 23, a CDRL2 comprising the amino acid sequence of SEQ ID NO: 24, and a CDRL3 comprising the amino acid sequence of SEQ ID NO: 25, and a lambda light chain comprising a CDRL1 comprising the amino acid sequence of SEQ ID NO: 131, a CDRL2 comprising the amino acid sequence of SEQ ID NO: 132, and a CDRL3 comprising the amino acid sequence of SEQ ID NO: 133.

In some embodiments, the AI3O25/N bispecific antibody has a heavy chain variable region comprising the amino acid sequence of SEQ ID NO: 4, a kappa light chain variable region comprising the amino acid sequence of SEQ ID NO: 80, and a lambda light chain variable region comprising the amino acid sequence of SEQ ID NO: 144.

In some embodiments, the AI3O25/N bispecific antibody has a heavy chain variable and constant region comprising the amino acid sequence of SEQ ID NO: 7, a kappa light chain comprising the amino acid sequence of SEQ ID NO: 99, and a lambda light chain comprising the amino acid sequence of SEQ ID NO: 149.

In some embodiments, the AI3O35/N bispecific antibody has a heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 23, a CDRL2 comprising the amino acid sequence of SEQ ID NO: 24, and a CDRL3 comprising the amino acid sequence of SEQ ID NO: 25, and a lambda light chain comprising a CDRL1 comprising the amino acid sequence of SEQ ID NO: 134, a CDRL2 comprising the amino acid sequence of SEQ ID NO: 135, and a CDRL3 comprising the amino acid sequence of SEQ ID NO: 136.

In some embodiments, the AI3O35/N bispecific antibody has a heavy chain variable region comprising the amino acid sequence of SEQ ID NO: 4, a kappa light chain variable region comprising the amino acid sequence of SEQ ID NO: 80, and a lambda light chain variable region comprising the amino acid sequence of SEQ ID NO: 145.

In some embodiments, the AI3O35/N bispecific antibody has a heavy chain variable and constant region comprising the amino acid sequence of SEQ ID NO: 7, a kappa light chain comprising the amino acid sequence of SEQ ID NO: 99, and a lambda light chain comprising the amino acid sequence of SEQ ID NO: 150.

In some embodiments, the AI3O38/N bispecific antibody has a heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 23, a CDRL2 comprising the amino acid sequence of SEQ ID NO: 24, and a CDRL3 comprising the amino acid sequence of SEQ ID NO: 25, and a lambda light chain comprising a CDRL1 comprising the amino acid sequence of SEQ ID NO: 137, a CDRL2 comprising the amino acid sequence of SEQ ID NO: 138, and a CDRL3 comprising the amino acid sequence of SEQ ID NO: 139.

In some embodiments, the AI3O38/N bispecific antibody has a heavy chain variable region comprising the amino acid sequence of SEQ ID NO: 4, a kappa light chain variable region comprising the amino acid sequence of SEQ ID NO: 80, and a lambda light chain variable region comprising the amino acid sequence of SEQ ID NO: 146.

In some embodiments, the AI3O38/N bispecific antibody has a heavy chain variable and constant region comprising the amino acid sequence of SEQ ID NO: 7, a kappa light chain comprising the amino acid sequence of SEQ ID NO: 99, and a lambda light chain comprising the amino acid sequence of SEQ ID NO: 151.

In some embodiments, the AI3O41/N bispecific antibody has a heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 23, a CDRL2 comprising the amino acid sequence of SEQ ID NO: 24, and a CDRL3 comprising the amino acid sequence of SEQ ID NO: 25, and a lambda light chain comprising a CDRL1 comprising the amino acid sequence of SEQ ID NO: 140, a CDRL2 comprising the amino acid sequence of SEQ ID NO: 141, and a CDRL3 comprising the amino acid sequence of SEQ ID NO: 142.

In some embodiments, the AI3O41/N bispecific antibody has a heavy chain variable region comprising the amino acid sequence of SEQ ID NO: 4, a kappa light chain variable region comprising the amino acid sequence of SEQ ID NO: 80, and a lambda light chain variable region comprising the amino acid sequence of SEQ ID NO: 147.

In some embodiments, the AI3O41/N bispecific antibody has a heavy chain variable and constant region comprising the amino acid sequence of SEQ ID NO: 7, a kappa light chain comprising the amino acid sequence of SEQ ID NO: 99, and a lambda light chain comprising the amino acid sequence of SEQ ID NO: 152.

In some embodiments, the AI5O30/N bispecific antibody has a heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 26, a CDRL2 comprising the amino acid sequence of SEQ ID NO: 27, and a CDRL3 comprising the amino acid sequence of SEQ ID NO: 28, and a lambda light chain comprising a CDRL1 comprising the amino acid sequence of SEQ ID NO: 128, a CDRL2 comprising the amino acid sequence of SEQ ID NO: 129, and a CDRL3 comprising the amino acid sequence of SEQ ID NO: 130.

In some embodiments, the AI5O30/N bispecific antibody has a heavy chain variable region comprising the amino acid sequence of SEQ ID NO: 4, a kappa light chain variable region comprising the amino acid sequence of SEQ ID NO: 81, and a lambda light chain variable region comprising the amino acid sequence of SEQ ID NO: 143.

In some embodiments, the AI5O30/N bispecific antibody has a heavy chain variable and constant region comprising the amino acid sequence of SEQ ID NO: 7, a kappa light chain comprising the amino acid sequence of SEQ ID NO: 100, and a lambda light chain comprising the amino acid sequence of SEQ ID NO: 148.

In some embodiments, the AI5O25/N bispecific antibody has a heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 26, a CDRL2 comprising the amino acid sequence of SEQ ID NO: 27, and a CDRL3 comprising the amino acid sequence of SEQ ID NO: 28, and a lambda light chain comprising a CDRL1 comprising the amino acid sequence of SEQ ID NO: 131, a CDRL2 comprising the amino acid sequence of SEQ ID NO: 132, and a CDRL3 comprising the amino acid sequence of SEQ ID NO: 133.

In some embodiments, the AI5O25/N bispecific antibody has a heavy chain variable region comprising the amino acid sequence of SEQ ID NO: 4, a kappa light chain variable region comprising the amino acid sequence of SEQ ID NO: 81, and a lambda light chain variable region comprising the amino acid sequence of SEQ ID NO: 144.

In some embodiments, the AI5O25/N bispecific antibody has a heavy chain variable and constant region comprising the amino acid sequence of SEQ ID NO: 7, a kappa light chain comprising the amino acid sequence of SEQ ID NO: 100, and a lambda light chain comprising the amino acid sequence of SEQ ID NO: 149.

In some embodiments, the AI5O35/N bispecific antibody has a heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 26, a CDRL2 comprising the amino acid sequence of SEQ ID NO: 27, and a CDRL3 comprising the amino acid sequence of SEQ ID NO: 28, and a lambda light chain comprising a CDRL1 comprising the amino acid sequence of SEQ ID NO: 134, a CDRL2 comprising the amino acid sequence of SEQ ID NO: 135, and a CDRL3 comprising the amino acid sequence of SEQ ID NO: 136.

In some embodiments, the AI5O35/N bispecific antibody has a heavy chain variable region comprising the amino acid sequence of SEQ ID NO: 4, a kappa light chain variable region comprising the amino acid sequence of SEQ ID NO: 81, and a lambda light chain variable region comprising the amino acid sequence of SEQ ID NO: 145.

In some embodiments, the AI5O35/N bispecific antibody has a heavy chain variable and constant region comprising the amino acid sequence of SEQ ID NO: 7, a kappa light chain comprising the amino acid sequence of SEQ ID NO: 100, and a lambda light chain comprising the amino acid sequence of SEQ ID NO: 150.

In some embodiments, the AI5O38/N bispecific antibody has a heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 26, a CDRL2 comprising the amino acid sequence of SEQ ID NO: 27, and a CDRL3 comprising the amino acid sequence of SEQ ID NO: 28, and a lambda light chain comprising a CDRL1 comprising the amino acid sequence of SEQ ID NO: 137, a CDRL2 comprising the amino acid sequence of SEQ ID NO: 138, and a CDRL3 comprising the amino acid sequence of SEQ ID NO: 139.

In some embodiments, the AI5O38/N bispecific antibody has a heavy chain variable region comprising the amino acid sequence of SEQ ID NO: 4, a kappa light chain variable region comprising the amino acid sequence of SEQ ID NO: 81, and a lambda light chain variable region comprising the amino acid sequence of SEQ ID NO: 146.

In some embodiments, the AI5O38/N bispecific antibody has a heavy chain variable and constant region comprising the amino acid sequence of SEQ ID NO: 7, a kappa light chain comprising the amino acid sequence of SEQ ID NO: 100, and a lambda light chain comprising the amino acid sequence of SEQ ID NO: 151.

In some embodiments, the AI5O41/N bispecific antibody has a heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 26, a CDRL2 comprising the amino acid sequence of SEQ ID NO: 27, and a CDRL3 comprising the amino acid sequence of SEQ ID NO: 28, and a lambda light chain comprising a CDRL1 comprising the amino acid sequence of SEQ ID NO: 140, a CDRL2 comprising the amino acid sequence of SEQ ID NO: 141, and a CDRL3 comprising the amino acid sequence of SEQ ID NO: 142.

In some embodiments, the AI5O41/N bispecific antibody has a heavy chain variable region comprising the amino acid sequence of SEQ ID NO: 4, a kappa light chain variable region comprising the amino acid sequence of SEQ ID NO: 81, and a lambda light chain variable region comprising the amino acid sequence of SEQ ID NO: 147.

In some embodiments, the AI5O41/N bispecific antibody has a heavy chain variable and constant region comprising the amino acid sequence of SEQ ID NO: 7, a kappa light chain comprising the amino acid sequence of SEQ ID NO: 100, and a lambda light chain comprising the amino acid sequence of SEQ ID NO: 152.

In some embodiments, the AI1O30/N bispecific antibody has a heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 38, a CDRL2 comprising the amino acid sequence of SEQ ID NO: 39, and a CDRL3 comprising the amino acid sequence of SEQ ID NO: 40, and a lambda light chain comprising a CDRL1 comprising the amino acid sequence of SEQ ID NO: 140, a CDRL2 comprising the amino acid sequence of SEQ ID NO: 129, and a CDRL3 comprising the amino acid sequence of SEQ ID NO: 130.

In some embodiments, the AI1O30/N bispecific antibody has a heavy chain variable region comprising the amino acid sequence of SEQ ID NO: 4, a kappa light chain variable region comprising the amino acid sequence of SEQ ID NO: 85, and a lambda light chain variable region comprising the amino acid sequence of SEQ ID NO: 143.

In some embodiments, the AI1O30/N bispecific antibody has a heavy chain variable and constant region comprising the amino acid sequence of SEQ ID NO: 7, a kappa light chain comprising the amino acid sequence of SEQ ID NO: 104, and a lambda light chain comprising the amino acid sequence of SEQ ID NO: 148.

In some embodiments, the AI1O25/N bispecific antibody has a heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 38, a CDRL2 comprising the amino acid sequence of SEQ ID NO: 39, and a CDRL3 comprising the amino acid sequence of SEQ ID NO: 40, and a lambda light chain comprising a CDRL1 comprising the amino acid sequence of SEQ ID NO: 131, a CDRL2 comprising the amino acid sequence of SEQ ID NO: 132, and a CDRL3 comprising the amino acid sequence of SEQ ID NO: 133.

In some embodiments, the AI1O25/N bispecific antibody has a heavy chain variable region comprising the amino acid sequence of SEQ ID NO: 4, a kappa light chain variable region comprising the amino acid sequence of SEQ ID NO: 85, and a lambda light chain variable region comprising the amino acid sequence of SEQ ID NO: 144.

In some embodiments, the AI1O25/N bispecific antibody has a heavy chain variable and constant region comprising the amino acid sequence of SEQ ID NO: 7, a kappa light chain comprising the amino acid sequence of SEQ ID NO: 104, and a lambda light chain comprising the amino acid sequence of SEQ ID NO: 149.

In some embodiments, the AI1O35/N bispecific antibody has a heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 38, a CDRL2 comprising the amino acid sequence of SEQ ID NO: 39, and a CDRL3 comprising the amino acid sequence of SEQ ID NO: 40, and a lambda light chain comprising a CDRL1 comprising the amino acid sequence of SEQ ID NO: 134, a CDRL2 comprising the amino acid sequence of SEQ ID NO: 135, and a CDRL3 comprising the amino acid sequence of SEQ ID NO: 136.

In some embodiments, the AI1O35/N bispecific antibody has a heavy chain variable region comprising the amino acid sequence of SEQ ID NO: 4, a kappa light chain variable region comprising the amino acid sequence of SEQ ID NO: 85, and a lambda light chain variable region comprising the amino acid sequence of SEQ ID NO: 145.

In some embodiments, the AI1O35/N bispecific antibody has a heavy chain variable and constant region comprising the amino acid sequence of SEQ ID NO: 7, a kappa light chain comprising the amino acid sequence of SEQ ID NO: 104, and a lambda light chain comprising the amino acid sequence of SEQ ID NO: 150.

In some embodiments, the AI1O38/N bispecific antibody has a heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 38, a CDRL2 comprising the amino acid sequence of SEQ ID NO: 39, and a CDRL3 comprising the amino acid sequence of SEQ ID NO: 40, and a lambda light chain comprising a CDRL1 comprising the amino acid sequence of SEQ ID NO: 137, a CDRL2 comprising the amino acid sequence of SEQ ID NO: 138, and a CDRL3 comprising the amino acid sequence of SEQ ID NO: 139.

In some embodiments, the AI1O38/N bispecific antibody has a heavy chain variable region comprising the amino acid sequence of SEQ ID NO: 4, a kappa light chain variable region comprising the amino acid sequence of SEQ ID NO: 85, and a lambda light chain variable region comprising the amino acid sequence of SEQ ID NO: 146.

In some embodiments, the AI1O38/N bispecific antibody has a heavy chain variable and constant region comprising the amino acid sequence of SEQ ID NO: 7, a kappa light chain comprising the amino acid sequence of SEQ ID NO: 104, and a lambda light chain comprising the amino acid sequence of SEQ ID NO: 151.

In some embodiments, the AI1O41/N bispecific antibody has a heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 38, a CDRL2 comprising the amino acid sequence of SEQ ID NO: 39, and a CDRL3 comprising the amino acid sequence of SEQ ID NO: 40, and a lambda light chain comprising a CDRL1 comprising the amino acid sequence of SEQ ID NO: 140, a CDRL2 comprising the amino acid sequence of SEQ ID NO: 141, and a CDRL3 comprising the amino acid sequence of SEQ ID NO: 142.

In some embodiments, the AI1O41/N bispecific antibody has a heavy chain variable region comprising the amino acid sequence of SEQ ID NO: 4, a kappa light chain variable region comprising the amino acid sequence of SEQ ID NO: 85, and a lambda light chain variable region comprising the amino acid sequence of SEQ ID NO: 147.

In some embodiments, the AI1O41/N bispecific antibody has a heavy chain variable and constant region comprising the amino acid sequence of SEQ ID NO: 7, a kappa light chain comprising the amino acid sequence of SEQ ID NO: 104, and a lambda light chain comprising the amino acid sequence of SEQ ID NO: 152.

In some embodiments, the AI13030/N bispecific antibody has a heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 38, a CDRL2 comprising the amino acid sequence of SEQ ID NO: 39, and a CDRL3 comprising the amino acid sequence of SEQ ID NO: 40, and a lambda light chain comprising a CDRL1 comprising the amino acid sequence of SEQ ID NO: 128, a CDRL2 comprising the amino acid sequence of SEQ ID NO: 129, and a CDRL3 comprising the amino acid sequence of SEQ ID NO: 130.

In some embodiments, the AI13030/N bispecific antibody has a heavy chain variable region comprising the amino acid sequence of SEQ ID NO: 4, a kappa light chain variable region comprising the amino acid sequence of SEQ ID NO: 88, and a lambda light chain variable region comprising the amino acid sequence of SEQ ID NO: 143.

In some embodiments, the AI13030/N bispecific antibody has a heavy chain variable and constant region comprising the amino acid sequence of SEQ ID NO: 7, a kappa light chain comprising the amino acid sequence of SEQ ID NO: 107, and a lambda light chain comprising the amino acid sequence of SEQ ID NO: 148.

In some embodiments, the AI13025/N bispecific antibody has a heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 47, a CDRL2 comprising the amino acid sequence of SEQ ID NO: 48, and a CDRL3 comprising the amino acid sequence of SEQ ID NO: 49, and a lambda light chain comprising a CDRL1 comprising the amino acid sequence of SEQ ID NO: 131, a CDRL2 comprising the amino acid sequence of SEQ ID NO: 132, and a CDRL3 comprising the amino acid sequence of SEQ ID NO: 133.

In some embodiments, the AI13025/N bispecific antibody has a heavy chain variable region comprising the amino acid sequence of SEQ ID NO: 4, a kappa light chain variable region comprising the amino acid sequence of SEQ ID NO: 88, and a lambda light chain variable region comprising the amino acid sequence of SEQ ID NO: 144.

In some embodiments, the AI13025/N bispecific antibody has a heavy chain variable and constant region comprising the amino acid sequence of SEQ ID NO: 7, a kappa light chain comprising the amino acid sequence of SEQ ID NO: 107, and a lambda light chain comprising the amino acid sequence of SEQ ID NO: 149.

In some embodiments, the AI13035/N bispecific antibody has a heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 47, a CDRL2 comprising the amino acid sequence of SEQ ID NO: 48, and a CDRL3 comprising the amino acid sequence of SEQ ID NO: 49, and a lambda light chain comprising a CDRL1 comprising the amino acid sequence of SEQ ID NO: 134, a CDRL2 comprising the amino acid sequence of SEQ ID NO: 135, and a CDRL3 comprising the amino acid sequence of SEQ ID NO: 136.

In some embodiments, the AI13035/N bispecific antibody has a heavy chain variable region comprising the amino acid sequence of SEQ ID NO: 4, a kappa light chain variable region comprising the amino acid sequence of SEQ ID NO: 88, and a lambda light chain variable region comprising the amino acid sequence of SEQ ID NO: 145.

In some embodiments, the AI13035/N bispecific antibody has a heavy chain variable and constant region comprising the amino acid sequence of SEQ ID NO: 7, a kappa light chain comprising the amino acid sequence of SEQ ID NO: 107, and a lambda light chain comprising the amino acid sequence of SEQ ID NO: 150.

In some embodiments, the AI13038/N bispecific antibody has a heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 47, a CDRL2 comprising the amino acid sequence of SEQ ID NO: 48, and a CDRL3 comprising the amino acid sequence of SEQ ID NO: 49, and a lambda light chain comprising a CDRL1 comprising the amino acid sequence of SEQ ID NO: 137, a CDRL2 comprising the amino acid sequence of SEQ ID NO: 138, and a CDRL3 comprising the amino acid sequence of SEQ ID NO: 139.

In some embodiments, the AI13038/N bispecific antibody has a heavy chain variable region comprising the amino acid sequence of SEQ ID NO: 4, a kappa light chain variable region comprising the amino acid sequence of SEQ ID NO: 88, and a lambda light chain variable region comprising the amino acid sequence of SEQ ID NO: 146.

In some embodiments, the AI13038/N bispecific antibody has a heavy chain variable and constant region comprising the amino acid sequence of SEQ ID NO: 7, a kappa light chain comprising the amino acid sequence of SEQ ID NO: 107, and a lambda light chain comprising the amino acid sequence of SEQ ID NO: 151.

In some embodiments, the AI13041/N bispecific antibody has a heavy chain comprising a CDRH1 comprising the amino acid sequence of SEQ ID NO: 1, a CDRH2 comprising the amino acid sequence of SEQ ID NO: 2, a CDRH3 comprising the amino acid sequence of SEQ ID NO: 3, a kappa light chain comprising a CDRL1 comprising the amino acid sequence of SEQ ID NO: 47, a CDRL2 comprising the amino acid sequence of SEQ ID NO: 48, and a CDRL3 comprising the amino acid sequence of SEQ ID NO: 49, and a lambda light chain comprising a CDRL1 comprising the amino acid sequence of SEQ ID NO: 140, a CDRL2 comprising the amino acid sequence of SEQ ID NO: 141, and a CDRL3 comprising the amino acid sequence of SEQ ID NO: 142.

In some embodiments, the AI13041/N bispecific antibody has a heavy chain variable region comprising the amino acid sequence of SEQ ID NO: 4, a kappa light chain variable region comprising the amino acid sequence of SEQ ID NO: 88, and a lambda light chain variable region comprising the amino acid sequence of SEQ ID NO: 147.

In some embodiments, the AI13041/N bispecific antibody has a heavy chain variable and constant region comprising the amino acid sequence of SEQ ID NO: 7, a kappa light chain comprising the amino acid sequence of SEQ ID NO: 107, and a lambda light chain comprising the amino acid sequence of SEQ ID NO: 152.

Methods of Use

Therapeutic formulations of the invention, which include the bsAbs of the invention, are used to treat or alleviate a symptom associated with a cancer, such as, by way of non-limiting example, leukemias, lymphomas, breast cancer, colon cancer, ovarian cancer, bladder cancer, prostate cancer, glioma, lung & bronchial cancer, colorectal cancer, pancreatic cancer, esophageal cancer, liver cancer, urinary bladder cancer, kidney and renal pelvis cancer, oral cavity & pharynx cancer, uterine corpus cancer, and/or melanoma. The present invention also provides methods of treating or alleviating a symptom associated with a cancer. A therapeutic regimen is carried out by identifying a subject, e.g., a human patient suffering from (or at risk of developing) a cancer, using standard methods.

Efficaciousness of treatment is determined in association with any known method for diagnosing or treating the particular immune-related disorder. Alleviation of one or more symptoms of the immune-related disorder indicates that the antibody confers a clinical benefit.

Pharmaceutical Compositions

The antibodies of the invention (also referred to herein as “active compounds”), and derivatives, fragments, analogs and homologs thereof, can be incorporated into pharmaceutical compositions suitable for administration. Such compositions typically comprise the antibody and a pharmaceutically acceptable carrier. As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration.

### Definitions

As used herein, including the appended claims, the singular forms of words such as “a,” “an,” and “the,” include their corresponding plural references unless the context clearly dictates otherwise.

As used herein, “mAb V8” refers to “Molecule 11U: CD28(SA_Variant 8) (PG-LALA), CD28 (SA_Variant 8) antibody in a huIgG1 PG-LALA isotype” as described in WO2020127618 (incorporated by reference in its entirety), comprising the amino acid sequences of SEQ ID NO:377 and SEQ ID NO:378 of WO2020127618.

As used herein, “mAb V15” refers to “Molecule 11W: CD28(SA_Variant 15) (PG-LALA), CD28 (SA_Variant 15) antibody in a huIgG1 PG-LALA isotype” as described in WO2020127618 (incorporated by reference in its entirety), comprising the amino acid sequences of SEQ ID NO:381 and SEQ ID NO:382 of WO2020127618.

As used herein, “CEA_CD28_V8” refers to “Molecule 11B: CEA (A5H1EL1D)-CD28 (SA Variant 8) 1+1 format, bispecific huIgG1 PGLALA CrossFab molecule with charged modifications in the CD28(SA_Variant 8) Fab fragment (knob) and VH/VL exchange in CEA(A5H1EL1D) Fab fragment (hole)” as described in WO2020127618 (incorporated by reference in its entirety), comprising the amino acid sequences of SEQ ID NOs: 351, 352, 355 and 356 of WO2020127618.

As used herein, “CEA_CD28_V15” refers to “Molecule 11C: CEA (A5H1EL1D)-CD28 (SA Variant 15) 1+1 format, bispecific huIgG1 PGLALA CrossFab molecule with charged modifications in the CD28(SA_Variant 15) Fab fragment (knob) and VH/VL exchange in CEA(A5H1EL1D) Fab fragment (hole)” as described in WO2020127618 (incorporated by reference in its entirety), comprising the amino acid sequences of SEQ ID NOs: 351, 352, 357 and 358 of WO2020127618.

As used herein, “mAb 14226P2” refers to “mAb14226P2 (CD28 Parental “A”)”, the anti-CD28 arm used for the generation of bs16429D (PSMA×CD28 “A”) as described in WO2019246514 (incorporated by reference in its entirety), that comprises the amino acid sequences of SEQ ID NOS: 81 and 83 of WO2019246514.

As used herein, “1a28/AC84/N” refers to a benchmark CEA×CD28 bsAb produced by pairing the anti-CD28 arm of mAb 14226P2 with the anti-CEA arm AC84 using the crossMAb/Knobs-Into-Hole technologies, and comprising the amino acid sequences of SEQ ID NOS: 122, 123, 124 and 125.

As used herein, “TGN1412”, refers to a superagonistic (SA) anti-huCD28 antibody in a human IgG4 isotype as described in WO2006050949, that comprises the amino acid sequences of SEQ ID NOS: 126 and 127.

As used herein, “HPN536 analog” refers to a trispecific MSLN×CD3×HSA molecule capable of redirecting T cells toward MSLN-positive target cells as described in WO2018209304 (incorporated by reference in its entirety), comprising the amino acid sequence of SEQ ID NOS: 100 of WO2018209304.

“Affinity” refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including KinExA and Biacore.

As used herein, the term “antibody” includes, but is not limited to, monoclonal antibodies, polyclonal antibodies, multispecific antibodies (e.g., bispecific antibodies), fully human antibodies, and chimeric antibodies.

As used herein, unless otherwise indicated, “antigen-binding fragment” refers to antigen-binding fragments of antibodies, i.e. antibody fragments that retain the ability to bind to the antigen bound by the full-length antibody, e.g. fragments that retain one or more CDR regions. Examples of antibody binding fragments include, but are not limited to, Fab, Fab′, F(ab′)₂, Fv fragments and individual antibody heavy chains or light chains, and individual heavy chain or light chain variable regions.

A “Fab fragment” is comprised of one light chain and the CH1 and variable regions of one heavy chain. The heavy chain of a Fab molecule cannot form a disulfide bond with another heavy chain molecule. A “Fab fragment” can be the product of papain cleavage of an antibody.

A “Fc” region contains two heavy chain fragments comprising the CH2 and CH3 domains of an antibody. The two heavy chain fragments are held together by two or more disulfide bonds and by hydrophobic interactions of the CH3 domains.

A “Fab′ fragment” contains one light chain and a portion or fragment of one heavy chain that contains the VH domain and the CH1 domain and also the region between the CH1 and CH2 domains, such that an interchain disulfide bond can be formed between the two heavy chains of two Fab′ fragments to form a F(ab′)2 molecule.

A “F(ab′)2 fragment” contains two light chains and two heavy chains containing a portion of the constant region between the CH1 and CH2 domains, such that an interchain disulfide bond is formed between the two heavy chains. A F(ab′)₂ fragment thus is composed of two Fab′ fragments that are held together by a disulfide bond between the two heavy chains. An “F(ab′)2 fragment” can be the product of pepsin cleavage of an antibody. The “Fv region” comprises the variable regions from both the heavy and light chains but lacks the constant regions.

“Isolated antibody” refers to the purification status and in such context means the molecule is substantially free of other biological molecules such as nucleic acids, proteins, lipids, carbohydrates, or other material such as cellular debris and growth media. Generally, the term “isolated” is not intended to refer to a complete absence of such material or to an absence of water, buffers, or salts, unless they are present in amounts that substantially interfere with experimental or therapeutic use of the binding compound as described herein.

The term “monoclonal antibody”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains that are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

The term “fully human antibody” refers to an antibody that comprises human immunoglobulin protein sequences only. A fully human antibody may contain murine carbohydrate chains if produced in a mouse, in a mouse cell, or in a hybridoma derived from a mouse cell. Similarly, “mouse antibody” refers to an antibody that comprises mouse immunoglobulin sequences only. Alternatively, a fully human antibody may contain rat carbohydrate chains if produced in a rat, in a rat cell, or in a hybridoma derived from a rat cell. Similarly, “rat antibody” refers to an antibody that comprises rat immunoglobulin sequences only.

In general, the basic “antibody” structural unit comprises a tetramer. In a monospecific antibody, each tetramer includes two identical pairs of polypeptide chains, each pair having one “light” (about 25 kDa) and one “heavy” chain (about 50-70 kDa). The amino-terminal portion of each chain includes a “variable region” or “variable domain” of about 100 to 110 or more amino acids primarily responsible for antigen recognition. The carboxy-terminal portion of the heavy chain may define a constant region primarily responsible for effector function.

Typically, human constant light chains are classified as kappa and lambda light chains. Furthermore, human constant heavy chains are typically classified as mu, delta, gamma, alpha, or epsilon, and define the antibody's isotype as IgM, IgD, IgG, IgA, and IgE, respectively. Subtypes of these IgG include, for example, IgG1 and IgG4.

“Variable region,” “variable domain,” “V region,” or “V chain” as used herein means the segment of IgG chains which is variable in sequence between different antibodies. A “variable region” of an antibody refers to the variable region of the antibody light chain or the variable region of the antibody heavy chain, either alone or in combination. The variable region of the heavy chain may be referred to as “VH.” The variable region of the light chain may be referred to as “VL.” Typically, the variable regions of both the heavy and light chains comprise three hypervariable regions, also called complementarity determining regions (CDRs), which are located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3, and FR4. The assignment of amino acids to each domain is, generally, in accordance with the definitions of Sequences of Proteins of Immunological Interest, Kabat, et al.; National Institutes of Health, Bethesda, Md.; 5th ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883.

A “CDR” refers to one of three hypervariable regions (H1, H2, or H3) within the non-framework region of the antibody V_{H} .beta.-sheet framework, or one of three hypervariable regions (L1, L2, or L3) within the non-framework region of the antibody VL beta.-sheet framework. Accordingly, CDRs are variable region sequences interspersed within the framework region sequences. CDR regions are well known to those skilled in the art and have been defined by, for example, Kabat as the regions of most hypervariability within the antibody variable domains. CDR region sequences also have been defined structurally by Chothia as those residues that are not part of the conserved .beta.-sheet framework, and thus are able to adapt to different conformation. Both terminologies are well recognized in the art. CDR region sequences have also been defined by AbM, Contact, and IMGT. The positions of CDRs within a canonical antibody variable region have been determined by comparison of numerous structures (Al-Lazikani et al., 1997, J. Mol. Biol. 273:927-48; Morea et al., 2000, Methods 20:267-79). Because the number of residues within a hypervariable region varies in different antibodies, additional residues relative to the canonical positions are conventionally numbered with a, b, c and so forth next to the residue number in the canonical variable region numbering scheme (Al-Lazikani et al., supra). Such nomenclature is similarly well known to those skilled in the art. Correspondence between the numbering system, including, for example, the Kabat numbering and the IMGT unique numbering system, is well known to one skilled in the art In some embodiments, the CDRs are as defined by the Kabat numbering system. In other embodiments, the CDRs are as defined by the IMGT numbering system. In yet other embodiments, the CDRs are as defined by the AbM numbering system. In still other embodiments, the CDRs are as defined by the Chothia numbering system. In yet other embodiments, the CDRs are as defined by the Contact numbering system.

Sequence identity refers to the degree to which the amino acids of two polypeptides are the same at equivalent positions when the two sequences are optimally aligned.

Sequence similarity includes identical residues and non-identical, biochemically related amino acids. Biochemically related amino acids that share similar properties and may be interchangeable are discussed above.

“Conservatively modified variants” or “conservative substitution” refers to substitutions of amino acids in a protein with other amino acids having similar characteristics (e.g. charge, side-chain size, hydrophobicity/hydrophilicity, backbone conformation and rigidity, etc.), such that the changes can frequently be made without altering the biological activity of the protein. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity.

The term “epitope,” as used herein, refers to an area or region on an antigen to which an antibody or antigen-binding fragment binds. Binding of an antibody or antigen-binding fragment thereof disclosed herein to an epitope means that the antibody or antigen-binding fragment thereof binds to one or more amino acid residues within the epitope.

“Isolated” nucleic acid molecule or polynucleotide means a DNA or RNA, e.g., of genomic, mRNA, cDNA, or synthetic origin or some combination thereof which is not associated with all or a portion of a polynucleotide in which the isolated polynucleotide is found in nature, or is linked to a polynucleotide to which it is not linked in nature. For purposes of this disclosure, it should be understood that “a polynucleotide comprising” (or the like) a particular nucleotide sequence does not encompass intact chromosomes. Isolated polynucleotides “comprising” specified nucleic acid sequences may include, in addition to the specified sequences, coding sequences for up to ten or even up to twenty or more other proteins or portions or fragments thereof or may include operably linked regulatory sequences that control expression of the coding region of the recited nucleic acid sequences, and/or may include vector sequences.

The phrase “control sequences” refers to polynucleotide sequences necessary or helpful for the expression of an operably linked coding sequence in a particular host organism. The control sequences that are suitable for prokaryotes, for example, include a promoter, optionally an operator sequence, and a ribosome binding site. Eukaryotic cells are known to use promoters, polyadenylation signals, and enhancers. In an embodiment of the invention, the polynucleotide is operably linked to a promoter such as a viral promoter, a CMV promoter, an SV40 promoter or a non-viral promoter or an elongation factor (EF)-1 promotor; and/or an intron.

A nucleic acid is “operably linked” when it is placed into a functional relationship with another polynucleotide. For example, DNA for a pre-sequence or secretory leader is operably linked to DNA for a polypeptide if it is expressed as a pre-protein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, but not always, “operably linked” means that the polynucleotide sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that not all progeny will have precisely identical DNA content, due to deliberate or inadvertent mutations. Mutant progeny that have the same function or biological activity as screened for in the originally transformed cell are included. Where distinct designations are intended, it will be clear from the context.

Host cells include eukaryotic and prokaryotic host cells, including mammalian cells. Host cells include, inter alia, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, 3T3 cells and HEK-293 cells. Mammalian host cells include human, mouse, rat, dog, monkey, pig, goat, bovine, horse and hamster cells. Other cell lines that may be used are insect cell lines (e.g., Spodoptera frugiperda or Trichoplusia ni), amphibian cells, bacterial cells, plant cells and fungal cells. Fungal cells include yeast and filamentous fungus cells including, for example, Pichia pastoris, Pichia finlandica, Pichia trehalophia, Pichia koclamae, Pichia membranaefaciens, Pichia minuta (Ogataea minuta, Pichia lindnen), Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Physcomitrella patens and Neurospora crassa. Pichia sp., any Saccharomyces sp., Hansenula polymorpha, any Kluyveromyces sp., Candida albicans, any Aspergillus sp., Trichoderma reesei, Chrysosporium lucknowense, any Fusarium sp., Yarrowia lipolytica, and Neurospora crassa. The present invention includes any host cell (e.g., a CHO cell or Pichia cell, e.g., Pichia pastoris) containing an anti-ILT4 antibody or antigen-binding fragment thereof or containing a polynucleotide encoding such an antibody or fragment or containing a vector that contains the polynucleotide.

“Treat” or “treating” means to administer antibodies or antigen-binding fragments thereof of the present invention, to a subject having one or more symptoms of a disease for which the antibodies and antigen-binding fragments are effective, e.g., in the treatment of a subject having cancer or an infectious disease, or being suspected of having cancer or infectious disease, for which the agent has therapeutic activity. Typically, the antibody or fragment is administered in an “effective amount” or “effective dose” which will alleviate one or more symptoms (e.g., of cancer or infectious disease) in the treated subject or population, whether by inducing the regression or elimination of such symptoms or by inhibiting the progression of such symptom(s), e.g., cancer symptoms such as tumor growth or metastasis, by any clinically measurable degree. The effective amount of the antibody or fragment may vary according to factors such as the disease stage, age, and weight of the patient, and the ability of the drug to elicit a desired response in the subject.

### EXAMPLES

### Example 1: Phage Display Selection of CD28 Fvs Using Human Scfv Libraries Containing Fixed Variable Heavy Domain

General procedures for construction and handling of human scFv libraries displayed on M13 bacteriophage are described in Vaughan et al., (Nat. Biotech. 1996, 14:309-314), hereby incorporated by reference in its entirety. The libraries for selection and screening encode scFv that all share the same VH domain and are solely diversified in the VL domain. Methods for the generation of fixed VH libraries and their use for the identification and assembly of bispecific antibodies are described in US 2012/0184716 and WO 2012/023053, each of which is hereby incorporated by reference in its entirety. The procedures to identify scFv binding to human CD28 (huCD28) are described below. Selections were performed in solution with biotinylated huCD28 protein and/or on cells expressing huCD28. Selection strategies included up to 4 round of selections (i) on the recombinant protein, (ii) alternating recombinant protein and cells, (iii) 2 rounds on the recombinant protein followed by 2 rounds on cells.

Protein Selections

Aliquots of scFv phage libraries were blocked with PBS containing 2% (w/v) skimmed milk. Blocked phages were first deselected on streptavidin/neutravidin magnetic beads (Dynabeads™ MyOne™ Streptavidin T1 Magnetic Beads or Sera-Mag SpeedBeads Neutravidin™ Coated Magnetic Particles) then pre-incubated with 200 nM, 100 nM, 50 nM or 5 nM of biotinylated recombinant human CD28 (CD8-H82E5, Acro Biosystems). The phage+antigen mix was then captured by blocked magnetic beads (the same kind used for the deselection) and washed five times with PBS/0.1% Tween® 20 and twice with PBS only. Phages were eluted with 1 mg/mL Trypsin and, after the addition of AEBSF to block trypsin activity, directly added to exponentially growing TG1 cells. An aliquot of the infected TG1 was serial diluted to titer the selection outputs. Outputs were then rescued and used for the next round of selection.

Cell Surface Selections

Phage containing supernatants were blocked with PBS containing 10% FBS. Blocked phages were first deselected on CD28NEG TIB-153 cells (ATCC TIB 153) and then selected on CD28POS Jurkat cells (Jurkat Clone E6-1, ATCC TIB 152). Cells were pelleted and washed five times with PBS containing 10% FBS follow by a single wash with PBS only. Phages were eluted with 1 mg/mL Trypsin and, after the addition of AEBSF to block trypsin activity, directly added to exponentially growing TG1 cells. An aliquot of the infected TG1 was serial diluted to titer the selection outputs. Outputs were then rescued and used for the next round of selection.

### Example 2: Screening for scFv Binding/Non-Binding to Human CD28

Screening of scFv for binding to CD28 was tested either by ELISA using biotinylated huCD28-His (or biotinylated huCEA_ECD as negative control) or by flow cytometry using CD28POS (Jurkat) and CD28NEG (TIB-153) cells.

For the binding ELISA, neutravidin-coated plates were blocked with 1% casein in PBS. Biotinylated huCD28-His and biotinylated huCEA_ECD were captured at 5 nM. Dilution of freshly prepared periplasmic extracts containing the selected scFvs were applied to the plates and detected using a combination of mouse anti-c-myc antibody and donkey anti mouse IgG HRP antibody. The OD at 450 nm generated following the addition of TMB was measured using a microplate spectrophotometer. Hits were classified as specific binders if unable to bind to the irrelevant huCEA protein and if the OD450 on huCD28 was at least 3 times higher than the background OD450.

For flow cytometry binding assays, cells were harvested, washed and distributed into V bottom 96 well plate at 150′000 or 200′000 cells/well. Dilution of freshly prepared periplasmic extracts containing the selected scFvs were pre-incubated with mouse anti-c-myc antibody and added to the cells. After incubation, cells were washed, incubated with a goat anti-mouse IgG-APC detection antibody, and analyzed in an iQUE3 screener equipment (Sartorious). Hits were classified as positive and specific if at least 5% of the cells were displaying a binding signal on CD28POS Jurkat cells 3 times greater than the GeoMFI of the background and if such signal was not observed on CD28NEG TIB-153 cells.

Positive and specific hits were sequenced following DNA extraction from single clones.

### Example 3: Fixed VH Candidates Reformatting into IgG and Transient Expression in Mammalian Cells

After screening and sequencing, scFv candidates with the desired binding properties were reformatted into IgG and expressed by transient transfection into PEAK cells. The VH and VL sequences of selected scFv were amplified with specific oligonucleotides and cloned into an expression vector containing the heavy and light chain constant regions. The expression vectors were verified by sequencing and transfected into mammalian cells using Lipofectamine 2000 (Thermo Fisher Scientific) according to manufacturer's instructions. Briefly, 4×10⁶ PEAK cells were cultured in T75 flasks in 25 ml culture media containing fetal bovine serum. Transfected cells were cultured for 5-6 days at 37° C., IgG production was quantified using an Octet RED96 instrument. The supernatant was harvested for IgG purification on FcXL affinity resin (Thermo Fisher Scientific) according to manufacturer's instructions. Briefly, supernatants from transfected cells were incubated overnight at 4° C. with an appropriate amount of FcXL resin. After resin wash with PBS, samples were loaded on Amicon Pro column and the IgG consequently eluted in 50 mM Glycine pH 3.5. The eluted IgG fraction was then dialyzed by Amicon 50 kDa against Histidine NaCl pH 6.0 buffer and the IgG content is quantified by absorption at 280 nm. Purity and IgG integrity were verified by electrophoresis using an Agilent Bioanalyzer 2100 according to manufacturer instructions (Agilent Technologies).

### Example 4: Binding of Anti-CD28 mAbs to CD28-Positive Jurkat Cells

The binding capacity of the anti-CD28 antibody arms of the invention, tested as bivalent mAbs, was assessed using CD28-positive Jurkat cells (Jurkat Clone E6-1, ATCC TIB 152) by flow cytometry.

Cells were harvested, checked for viability and counted. 200′000 cells were incubated for 15 minutes at 4° C. with increasing concentrations of the antibodies diluted in FACS buffer (PBS 2% BSA, 0.1% NaN3). Cells were washed twice with cold FACS buffer and re-incubated for further 15 minutes at 4° C. with a suitable anti-human IgG secondary antibody. Cells were washed twice with cold FACS buffer and resuspended in 150 μl FACS buffer with a compatible viability marker. Binding of antibodies to living cells was measured by flow cytometry using a Cytoflex Platform (Beckman Coulter). Data was analyzed with FlowJo™ v10 software (BD Life Sciences) and dose-response binding curves were drawn using GraphPad Prism 9 software.

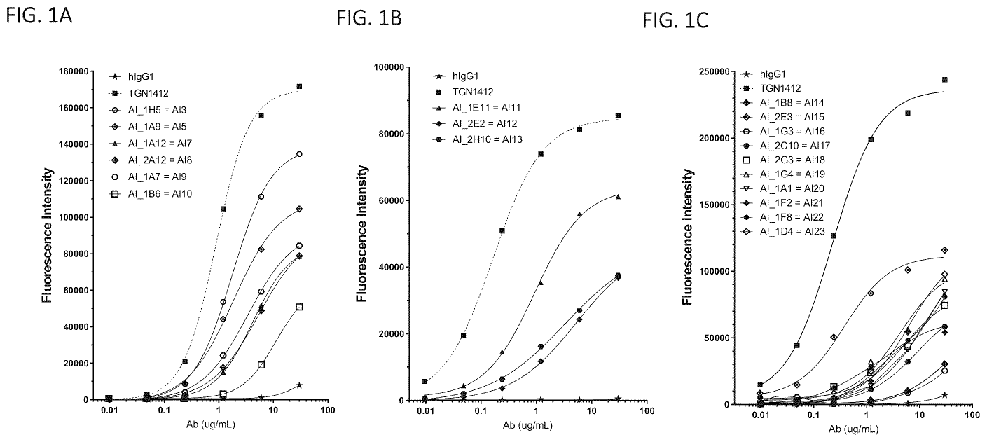

The resulting binding profile of a selection of κλ-body compatible anti CD28 arms is shown on FIGS. 1A-1C. The superagonist anti CD28 mAb TGN1412 and an irrelevant hIgG1 mAb served as positive and isotype control, respectively, and were used in all shown experiments for comparison. The wide spectrum of binding curves shown on the three graphs, representing anti-CD28 candidates identified during successive phage display campaigns, highlights the fact that a panel of anti-CD28 antibody arms with different binding affinities for CD28 was identified by phage display.

### Example 5: Expression and Purification of Bispecific Antibodies Carrying a Lambda and a Kappa Light Chain

The simultaneous expression of one heavy chain and two lights chain in the same cell can lead to the assembly of three different antibodies. Simultaneous expression can be achieved in different ways such as that the transfection of multiple vectors expressing one of the chains to be co-expressed or by using vectors that drive multiple gene expression.

Here, the two light chains were cloned into the vector pNovi κHλ that was previously generated to allow for the co-expression of one heavy chain, one Kappa light chain and one Lambda light chain as described in US20120184716 and WO2012023053, each of which is hereby incorporated by reference in its entirety. The expression of the three genes is driven by human cytomegalovirus promoters (hCMV) and the vector also contains a glutamine synthetase gene (GS) that enables the selection and establishment of stable cell lines. The common VH and the VL genes of the anti-CD28 IgG and of the anti-CEA IgG were cloned in the vector pNovi κHλ, for transient expression in mammalian cells. Expi293 cells were cultured in suspension in an appropriate Erlenmeyer flask with suitable number of cells and culture medium volume. Plasmid DNA was transfected into Expi293 cells using PEI. Antibody concentration in the supernatant of transfected cells was measured during the production using an Octet RED96. According to antibody concentration, supernatants were harvested 5 to 7 days after transfection and clarified by filtration after addition of diatomaceous earth (Sartorius). The purification was based on a three-step purification process. First, the CaptureSelect™ FcXL affinity matrix (Thermo Fisher Scientific) was washed with PBS and then added in the clarified supernatant. After incubation overnight at +4° C. and 20 rpm, supernatants were centrifuged at 2000 g for 10 min, flow through was stored and resin were washed twice with PBS. Then, the resin was transferred on Amicon Pro columns and a solution containing 50 mM glycine at pH 3.5 was used for elution. Several elution fractions were generated, neutralized with Tris-HCl pH7.4 and pooled. The pool containing total human IgGs (the bispecific and the two monospecific antibodies) was quantified using a Nanodrop spectrophotometer (NanoDrop Technologies). A small aliquot was stored for further analysis and the remaining sample was incubated for 30 min at RT and 20 rpm with the appropriate volume of CaptureSelect™ KappaXL affinity matrix (Thermo Fisher Scientific). Resin recovery and wash, elution and neutralization steps were performed as described above. The last affinity purification step was performed using the CaptureSelect™ lambda Fab affinity matrix (Thermo Fisher Scientific) applying the same process as for the kappa purification step. Alternatively, the purification was based on a two-step purification process, where only the CaptureSelect™ KappaXL affinity matrix and the CaptureSelect™ lambda Fab affinity matrix were used. All elution fractions were pooled and desalted against His-NaCl pH 6.0 formulation buffer using 50 kDa Amicon Ultra centrifugal filter units (Merck Millipore). The final product was quantified using the Nanodrop.

Purified bispecific antibodies were analyzed by electrophoresis in denaturing and reducing conditions using an Agilent 2100 Bioanalyzer with the Protein 80 kit as described by the manufacturer (Agilent Technologies). The aggregate level was determined by SEC-UPLC. All samples were tested for endotoxin contamination using the Limulus Amebocyte Lysate test (LAL; Charles River Laboratories). Table 2 summarizes the CEA×CD28 and MSLN×CD28 κλ-bispecific antibodies that were generated.

### Example 6: In Vitro Characterization of CEA×CD28 Bispecific Antibodies

Binding of CEA×CD28 bsAbs to CD28-positive Jurkat cells, CEA-positive LS174T cells or CEA and CD28 double-negative TIB153 cells was tested. To demonstrate the binding of CD28×CEA κλ-bodies to target cells, a series of experiments based on flow cytometry was performed.

Examples of cells that can be used include CEA-positive cell lines such as the colorectal adenocarcinoma cell line LS174T, CD28-positive cell lines such as the leukemic Jurkat T cells as well as CEA and CD28 double negative cell lines, such as the leukemic TIB153 cells. Cell staining and binding assessment was performed as described in Example 4.

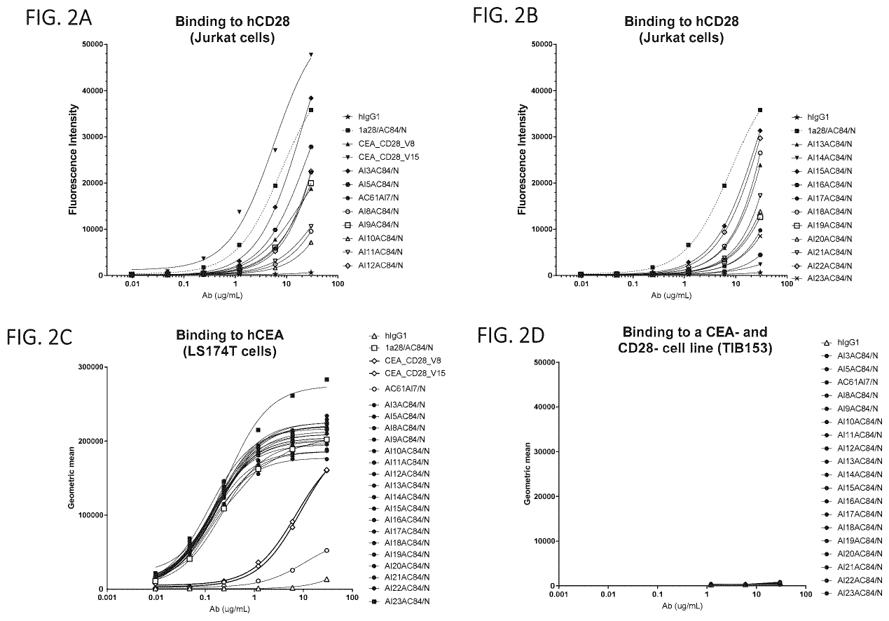

Binding curves of the CEA×CD28 bsAbs of the invention obtained using Jurkat, LS174T and TIB153 cells are shown in FIGS. 2A, 2B, 2C and 2D, respectively. On top of κλ-bodies, other antibodies were included as reference antibodies: hIgG1 denotes an irrelevant monoclonal antibody of hIgG1 Fc that served as isotype control; 1a28/AC84/N denotes a LALAPA-mutated huIgG1, 1+1 bispecific molecules based on the anti-CD28 antibody arm mAb14226P2 described in WO2019246514A1 using as targeting arm the anti-CEA arm AC84 described in WO2021110647 comprising the amino acid sequences of SEQ ID NOS: 122, 123, 124 and 125; while CEA_CD28_V8 and CEA_CD28_V15 correspond to CEA×CD28 bispecific antibodies described in WO2020127618, therein termed Molecule 111B and Molecule 11C, respectively.

Binding on Jurkat cells emphasizes the range of binding affinities for huCD28 of the selected anti-CD28 arms of the invention, in line with published anti-CD28 antibody arms or with lower binding affinity (FIGS. 2A and 2B). Binding on LS174T cells highlight how antibodies sharing the same anti-CEA arm bind similarly to cells, with all AC84-containing antibodies (high affinity anti-CEA arm) displaying equivalent dose-range profiles, followed by the two CEA_CD28 (V8 and V15), both containing the same mid-affinity anti-CEA arm, and lastly the only molecule based on the lower affinity anti-CEA arm AC61, AC61AI7/N (FIG. 2C). The absence of binding signal on TIB-153 suggest that all binding arms of the invention are specific for the designated target (FIG. 2D).

### Example 7: Wet and Dry Plate Coating T-Cell Proliferation Assay

To exclude anti-CD28 arms with superagonist activity, the capacity of CD28 bsAbs to induce T cell proliferation in the absence of signal 1 was assessed using wet and dry plate coating T-cell proliferation assays.

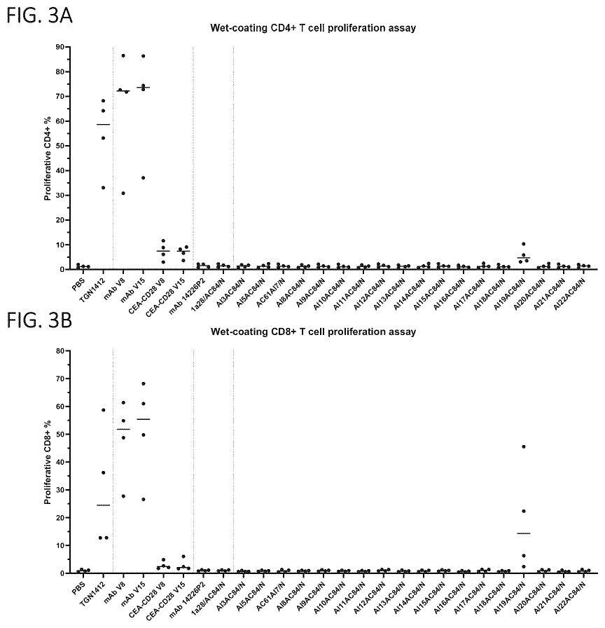

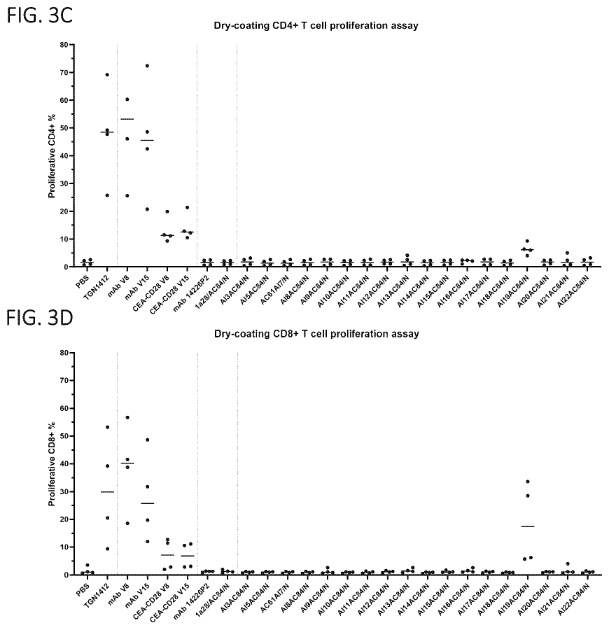

96-well polypropylene plates were coated overnight with antibodies diluted to 10 μg/ml in PBS, either at 4° C. with 100 μl of antibody solution (wet coating) or at room temperature, unsealed and in a class II laminar flow cabinet (to allow for the evaporation of the buffer=dry coating), with 50 μL of antibody solution. Following either coating procedure, plates were washed twice with PBS. In parallel, PBMCs isolated from buffy coat obtained from healthy donors were stained with CellTrace Violet Cell Proliferation Kit (ThermoFischer Scientific) according to the manufacturer's instructions. 100′000 stained PBMC cells were added to the 96-well plate in a final volume of 200 uL/well and incubated at 37° C.+5% C02 for 6 days. Cells were then harvested and stained for flow cytometry assessment using anti-CD4-APC (ThermoFischer, 17-0049-41) and anti-CD8-PerCP-Cy5.5 (BioLegend, 301032) as detailed in example 4 and 6b. The proliferation rate of living CD4+ and CD8+ T cells was calculated by measuring the levels of CellTrace Violet staining by flow cytometry using a CytoFLEX (Beckman Coulter) and results were evaluated by FlowJo software for both coating proceadures (FIG. 3A-3D). The CD28 SA antibody TGN1412 served as positive control, while the background proliferation rate of T cells was determined in the absence of any antibody (PBS only). The three CEA×CD28 bsAbs 1a28/AC84/N, CEA_CD28_V8 and CEA_CD28_V15 (described above) and their respective parental anti-CD28 monoclonal antibodies mAb 14226P2, mAb V8 and mAb V15 were included as reference antibodies.

Except for candidate AI19AC84/N, which induced mild but significant T cell proliferation, all other tested candidates led to CD4+ and CD8+ T cell proliferation in line with that induced by the vehicle control (PBS) with both coating procedures, thereby excluding unwanted superagonistic properties for these anti-CD28 arms. Candidate AI19AC84/N was precautionarily excluded from further analysis.

Contrary to all of the anti-CD28 arms of this invention (AI19 arm excepted), the anti-CD28 arms V8 and V15 described in WO2020127618 display some superagonistic activity, both as monoclonal antibodies (mAb V8 and mAb V15) and as bispecific CEA×CD28 antibodies (CEA×CD28 V8 and CEA×CD28 V15), as determined by the induction of proliferation of resting CD4+ and CD8+ T cells under both experimental conditions (wet and dry coating). No superagonistic activity was observed for the anti-CD28 arm 14226P2, neither as a monoclonal antibody (mAb 14226P2) nor as part of the bsAb 1a28/AC84/N.

### Example 8: T Cell Activation Bioassay (IL-2 Promoter)

Once a possible superagonistic activity excluded, the capacity of a selection of anti-CD28 arms to agonize the CD28 co-stimulatory receptor at the surface of T cell was assessed using an IL-2 reporter system in a bispecific format, and namely as CEA×CD28 bsAbs.

The T Cell Activation Bioassay (IL-2) kit developed by Promega (J1651) contains engineered Jurkat T cells that express a luciferase reporter gene driven by the IL-2 promoter. In presence of both CD3 and CD28 stimuli, receptor-mediated signaling induces activation of the IL-2 pathway, which results in luminescence of the engineered Jurkat T cells. Because CD28 bsAbs require both a T cell signal 1 provider and a TAA-positive target cell line to co-stimulate T cells, the assay was run in presence of CEA-positive MKN-45 cells and CEA×CD3 (a CEA×CD3 bispecific κλ-body comprising a common heavy chain HC of SEQ ID NO: 118, a kappa light chain of SEQ ID NO: 119, a lambda light chain of SEQ ID NO: 120, originally described in WO2021053587).

Briefly: 37,500 tumor target cells (MKN-45) were incubated for 6 h at 37° C. with 75000 IL-2 reporter cells (E:T=2:1) in presence of a fixed dose (1 nM) of CEA×CD3 alone or in combination with increasing concentrations of CEA×CD28 bispecific antibodies. Alternatively, CEA×CD3 was replaced by Y4L3-1/N, an untargeted anti-CD3 bsAb comprising a common heavy chain HC of SEQ ID NO: 118, a kappa light chain of SEQ ID NO: 121, a lambda light chain of SEQ ID NO: 120. Prior to the measurement, plates were equilibrated at room temperature for 15 min. 75 ul of substrate (Bio-Glo Reagent, Promega) were added to the cells and luminescence (Relative Luminescence Unit) was measured after 5-10 min of incubation at room temperature in the dark with a SpectraMax i3X luminometer (Molecular Devices).

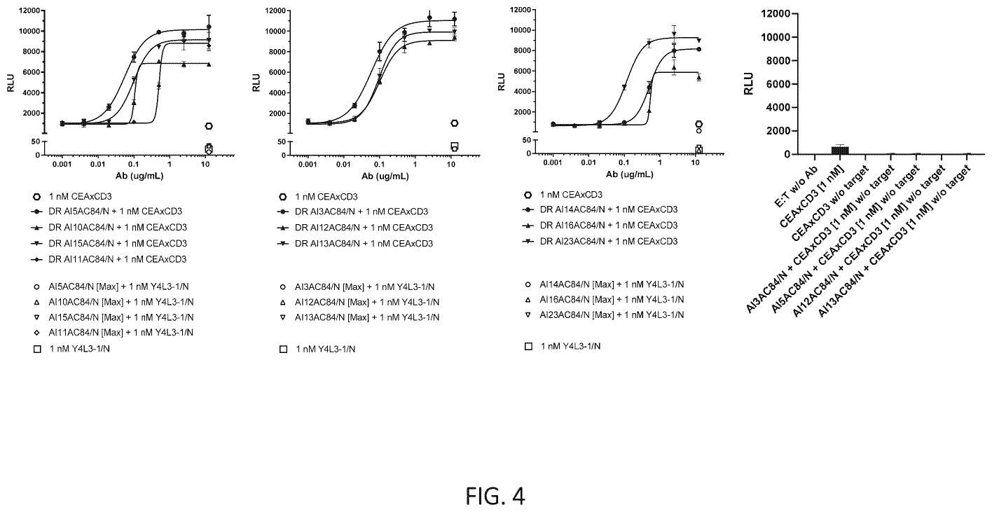

In presence of a basal CD3 stimulation (1 nM of the CEA×CD3), a concentration dependent rise in IL-2 promoter-mediated luminescence was observed with increasing CEA×CD28 bsAb concentrations, suggesting proper CD28 stimulation via the anti-CD28 arm of the bsAbs, resulting in enhanced T cell activation. No T cell activation could be observed in the absence of target cells or when CEA×CD3 was replaced by Y4L3-1/N, an untargeted CD3 bsAb unable to deliver T cell activation signal 1 (FIG. 4).

This reporter assay confirms that the CEA×CD28 bispecific antibodies of the invention can enhance T cell response only in presence of (1) a CEA-positive target cells and (2) primary T cell activation (signal 1).

### Example 9: T-Cell Dependent Cellular Cytotoxicity (TDCC) Mediated by CEA×CD28 Bispecific Antibodies

TDCC of CEA Positive and CEA Negative Cell Lines

The T-cell dependent cellular cytotoxicity (TDCC) of different CEA-positive and CEA-negative tumor cell lines induced by the CEA×CD28 bispecific antibodies of the present invention was assessed in combination with a CEA×CD3 bsAb using either human PBMCs or purified primary T-cell as effector cells.

Target cells are detached with trypsin or cell dissociation solution after two washes with PBS. After a centrifugation step, cells are resuspended in assay media, adjusted to the needed concentration, and plated in 96-well plates.

Effector cells can be either human peripheral blood mononuclear cells (PBMCs) or purified T-cells. PBMCs were isolated from buffy coats derived from healthy human donors using SepMate™ Tubes (Stemcell Technologies) with Lymphoprep™ buffer (Stemcell Technologies). If purified T-cells were used as effector cells, an extra purification step was performed, where T-cells were negatively isolated from PBMCs with the use of a T-cell immunomagnetic negative selection kit (STEMCELL Technologies).

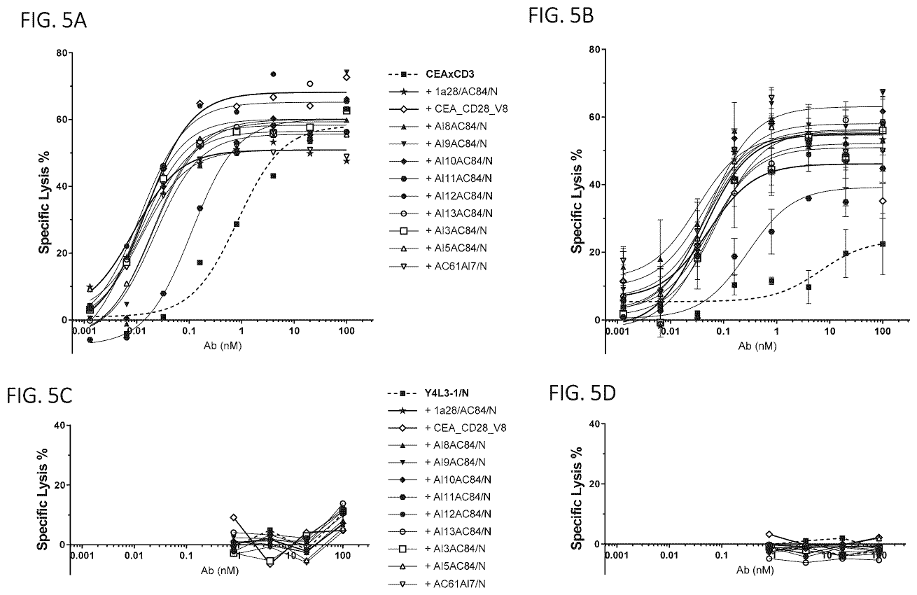

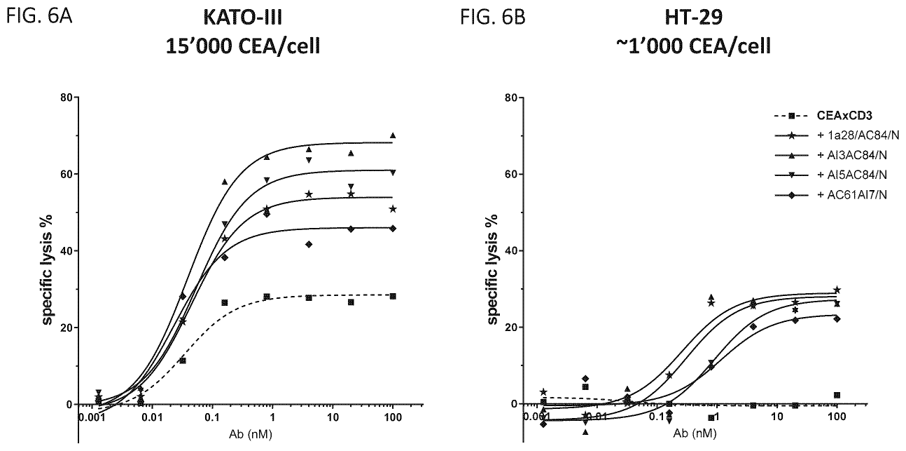

For the TDCC assay, when PBMCs were used as effector cells, these were added to target cells at final E:T ratio of 10:1 (unless stated otherwise); when purified T-cells were used, a final E:T ratio of 5:1 was used. A dose range of CEA×CD3 and a fixed dose of the CEA×CD28 antibodies of the invention (2.5, 1.0, 0.5 or 0.1 μg/mL) were added to the pre-plated target and effector cells. Alternatively, an untargeted CD3 bsAb (Y4L3-1/N) can be used instead of CEA×CD3. Target cell killing is assessed after either 24 h, 48 h, 72 h or 6 days of incubation at 37° C., 5% CO2 by quantifying (unless mentioned otherwise) the LDH released into the medium by apoptotic/necrotic cells (Cytotoxicity Detection KitPLUS (LDH), Roche). Maximal LDH release (=100% lysis) was obtained by incubating target cells with the lysis solution provided with the kit. Spontaneous LDH release (=0% lysis) refers to target cells co-incubated with effector cells without any antibody added. TDCC curves (FIGS. 5A-5D and 6A-6B) were plotted using GraphPad Prism 9. Alternatively, instead of LDH release, an assay based on ATP quantification was used (Promega, CellTiter-Glo® viability assay #G7571). In this case, plates were shaken on a rocking platform to resuspend the PBMCs in the supernatant. Then plates were washed twice with PBS to remove all PBMCs. In the meantime, the reagent from the kit was reconstituted and stabilized for 10 min at RT. Subsequently, 100 uL of the reconstituted reagent was added in each well and incubated for 10 min at RT, protected from direct light. The luminescence signal coming from the adenosine triphosphate (ATP) contained in the remaining live target cells in the well was measured using a microplate reader in luminescence mode of acquisition (Spectra i3Max). The percentage of specific lysis was calculated using the following equation: Specific lysis %=[1−(Sample value)/(Effector+Target)]×100, where Effector+Target correspond to the baseline value in the absence of antibodies. Specific lysis results were then analyzed with GraphPad Prism software.

CEA×CD28 bsAbs synergized with CEA×CD3 bsAb to kill CEA-positive LS174T target cells expressing 26′000 CEA/cell (FIGS. 5A-5D). The killing induced by two PBMC donors in presence of a dose range of CEA×CD3 and a 1 μg/mL fixed dose of the CEA×CD28 antibodies is shown. Synergy can be observed either as a lower EC50 or as a higher overall killing at the maximum concentration tested, or both. Importantly, if CEA×CD3 is replaced by Y4L3-1/N, an untargeted CD3 bsAb unable to deliver signal 1 to the effector cells, no killing is induced by the CEA×CD28 bsAbs, highlighting the importance of primary T cell stimulation (signal 1) for the activity of CD28 bsAbs. BsAbs 1a28/AC84/N and CEA_CD28_V8 (described above) were included as reference CEA×CD28 bsAbs and showed equivalent killing as the tested κλ-bodies, except for candidate AI11AC84/N, which consistently resulted in weaker activity in vitro.

The synergy between a CEA×CD3 bsAb and CEA×CD28 bsAbs is not cell line specific, as KATO-III and HT-29 cells, expressing 15′000 and ˜1′000 CEA/cell respectively, were also killed more efficiently by the CEA×CD3 (in dose response) and CEA×CD28 (at 2.5 μg/mL) bsAb combinations than by the CEA×CD3 bsAb alone (FIGS. 6A-5B). BsAb 1a28/AC84/N (described above) was included as reference CEA×CD28 bsAbs.

Up-Regulation of T-Cell Activation Markers Upon Killing of CEA-Expressing Tumor Cells Induced by the Combination of CEA×CD3 and CEA×CD28 bsAbs

Killing of CEA-positive tumor cells induced by CEA×CD3 bsAbs is based on T cell activation. The activation state of a T cell can be further increased by CD28 co-stimulation. The capacity of CEA×CD28 κλ-bodies to enhance T cell activation in presence of a proper signal 1 was thus quantified by flow cytometry using antibodies recognizing specific T cell activation markers such as CD69 (early activation marker) or CD25 (late activation marker).

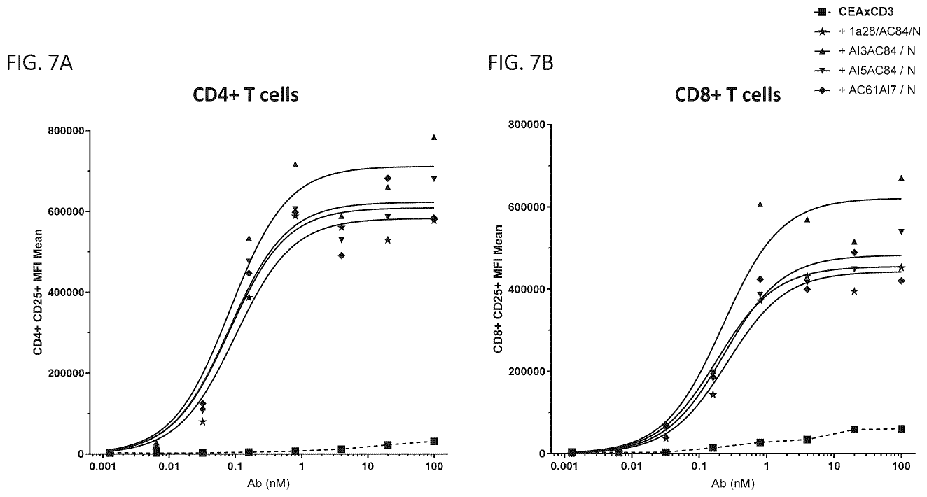

To assess the activation state of T cells at the end of a killing assay (detailed in example 9a), the following procedure was followed: floating cells (which include both CD4+ and CD8+ T-cells) were transferred into a new V-bottom 96-well plates. The supernatant was removed by centrifugation and cells were washed twice with cold FACS buffer (PBS 2% BSA, 0.1% NaN3) before being incubated for 15 minutes at 4° C. with Fc-block reagent (BD Biosciences). After two washing with FACS buffer, cells were incubated for 15 minutes at 4° C. with following antibodies: anti-CD69-FITC (BioLegend), anti-CD8-PerCP-Cy5.5 (BioLegend), anti-CD25-PE (BioLegend) and anti-CD4-APC (ThermoFisher). Cells were washed and analyzed by flow cytometry using a Cytoflex Platform (Beckman Coulter). Data was analyzed using FlowJo™ v10 software (BD Life Sciences). Results of an experiment where LS174T cells were co-cultured with PBMC for 72 h at a E:T ratio of 10:1, in presence of a dose range of CEA×CD3 and with a 1 ug/mL fixed dose of CEA×CD28 bsAb are shown in FIGS. 7A-7B.

T cell activation was measured by quantifying the late activation marker CD25 at the surface of both CD4+ and CD8+ T cells (FIGS. 7A and 7B, respectively). Compared to the single treatment with CEA×CD3, the combination of CEA×CD3 and CEA×CD28 bsAbs activated both CD4+ and CD8+ T cells to a greater extent, with much brighter CD25 staining for the combination treatments. BsAb 1a28/AC84/N (described above) was included as reference CEA×CD28 bsAb.

Effect of CD28 Costimulation Mediated by CEA×CD28 bsAbs on T Cell Proliferation in Presence of a CEA×CD3 bsAb.

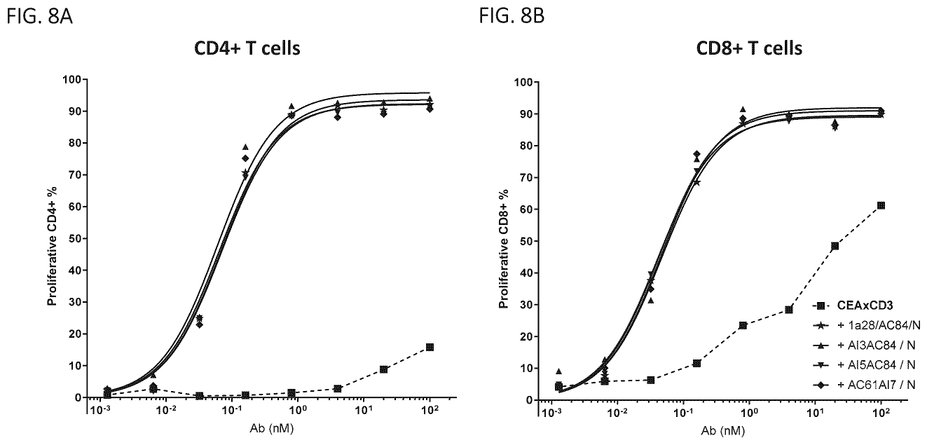

CEA×CD28 bsAbs were analyzed for their capability to enhance the effects of CEA×CD3 in term of induction of T cell proliferation in the presence of CEA-positive tumor target cells. Freshly isolated human PBMCs were stained with CellTrace Violet Cell Proliferation Kit (ThermoFischer Scientific) according to the manufacturer's instructions, washed and co-cultured for five to six days with target cells at a final E:T ratio of 10:1 (unless stated otherwise), in the presence of a dose range of CEA×CD3 and the indicated fixed dose of CEA×CD28 bsAbs. Following the co-culture, the effector cells were harvested, washed, stained with a suitable viability marker to exclude dead cells and with anti-CD4-APC (ThermoFischer, 17-0049-41) and anti-CD8-PerCP-Cy5.5 (BioLegend, 301032) to identify the populations of interest. The proliferation rate of T cells was calculated by measuring the levels of CellTrace Violet staining intensity on living CD4+ or CD8+ T cells by flow cytometry using a CytoFLEX (Beckman Coulter). Data was evaluated by FlowJo software and plotted using GraphPad Prism (FIGS. 8A-8B).

The percentage of proliferative CD4+ and CD8+ T cells is shown in FIGS. 8A and 8B, respectively. The capacity of a CEA×CD3 bsAb to induce T cell proliferation was greatly enhanced by the addition of 2.5 μg/mL CEA×CD28 bsAbs, and both CD4+ and CD8+ T cells reacted equally well to the combination treatments.

Cytokine Released in the Supernatant Upon Enhanced Killing of CEA-Expressing Tumor Cells by CD28 Costimulation with CEA×CD28 bsAbs

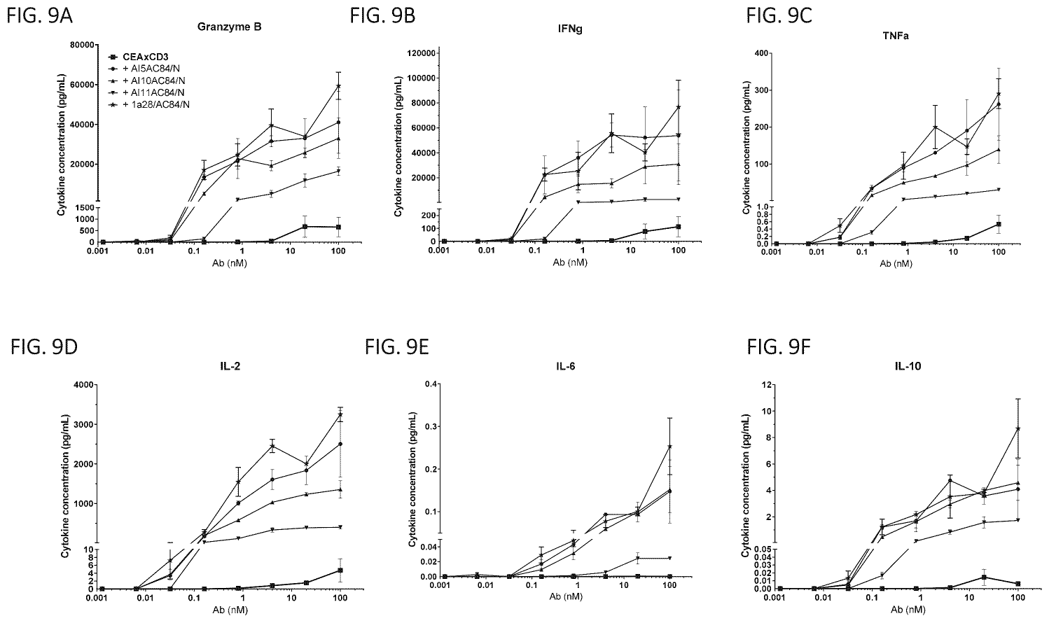

The capacity of the CD28 bispecific antibodies of this invention to enhance the release of cytokines by T cells upon killing of CEA-expressing tumor cells in presence of CEA×CD3 was assessed by quantifying selected cytokines in the supernatant at the end of a TDCC assay. Following the co-culture of CEA-positive target cells and T cell-containing PBMCs as described in Example 9, the culture supernatants were harvested by centrifugation and stored frozen at −80° until further analysis. Cytokines/enzymes such as Granzyme B, IL2, IL6, IL10, TNFα and IFNγ were quantified using the Mesoscale Discovery Platform by using multiplex kits. Results of an experiment where LS174T cells were co-cultured with PBMC at a E:T ratio of 5:1 for 72 h with a dose range of CEA×CD3 and a 1 μg/mL fixed dose of CEA×CD28 are shown in FIGS. 9A-9F. BsAb 1a28/AC84/N (described above) was included as reference CEA×CD28 bsAb.

Single agent treatment with CEA×CD3 resulted in moderate cytokine release by T cells, while combination treatment of CEA×CD3 with any of the tested CEA×CD28 bsAbs substantially increased all measured cytokines, reflecting the better activation and proliferation of T cells previously observed.

### Example 10: Evaluation of CD28 bsAb-Mediated T Cell Costimulation on the Anti-Tumor Activity of CEA×CD3 T-Cell Retargeting bsAb in Humanized Mouse Tumor Models

Anti-Tumor Activity of AI3AC84 N and AI5AC84 N in PBMC-Humanized Mouse Tumor Model

This efficacy study aimed at assessing the anti-tumoral effect of two CEA×CD28 κλ-bodies carrying anti-CD28 of intermediate affinity (AI3 and AI5), when combined with CEA×CD3.

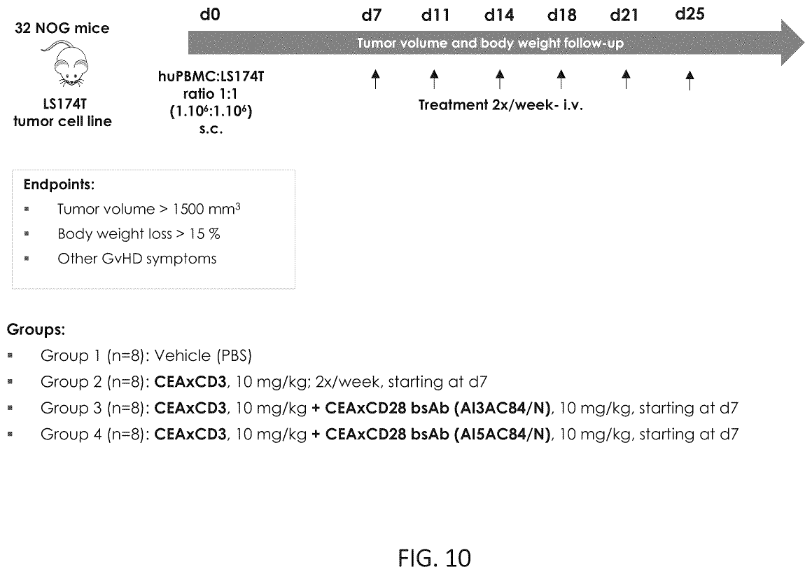

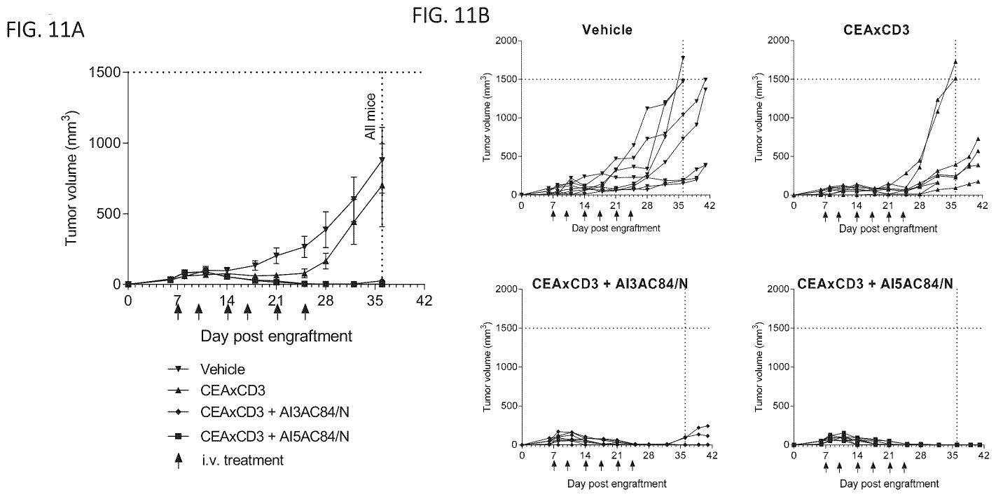

The experimental design is summarized in FIG. 10, where treatment groups and treatment regimens are also shown. Briefly: 1×10⁶ LS174T cells were co-engrafted subcutaneously with 1×10⁶ PBMCs (E:T=1:1) in NOG mice (NOD/Shi-scid/OL-2Rγnull mice, Taconic Biosciences) aged of 7-8 weeks and treatment was started 7 days post engraftment following randomization. CEA×CD3 and CEA×CD28 bsAbs were co-injected at 10 mg/kg twice weekly for a total of 6 injections. Mice were monitored for tumor development 2 to 3 times a week and tumors were measured by digital caliper until the endpoint of the experiment (tumor volume=1500 mm³ or onset of GvHD symptoms). Tumor volume was calculated using the formula (length×width²)×0.5. FIGS. 11A-11B shows the tumor growth kinetics (mean+SEM) per group (FIG. 11A) and mouse (FIG. 11B).

Only a mild inhibition of tumor growth over the vehicle group was observed in mice treated with CEA×CD3. However, the combinations of both AI3AC84/N and AI5AC84/N with CEA×CD3 induced complete tumor regression, with tumors that were undetectable to palpation on day 28 post-engraftment, when treatment was completed. The experiment was stopped on day 41: mice of the CEA×CD3+AI5AC84/N group were still tumor-free, contrary to the CEA×CD3+AI3AC84/N group, where 2 mice relapsed and had slowly growing tumors.

Anti-Tumor Activity of AI3AC84 N (Tested in a Dose Range) and AI10AC84 N in PBMC-Humanized Mouse Tumor Model

This efficacy study aimed at assessing the anti-tumoral effect of a dose range of a CEA×CD28 κλ-body carrying an anti-CD28 arm of intermediate affinity (AI3), and to compare it to the anti-tumoral effect of a CEA×CD28 κλ-body carrying an anti-CD28 arm of lower affinity (AI10), in combination with CEA×CD3.

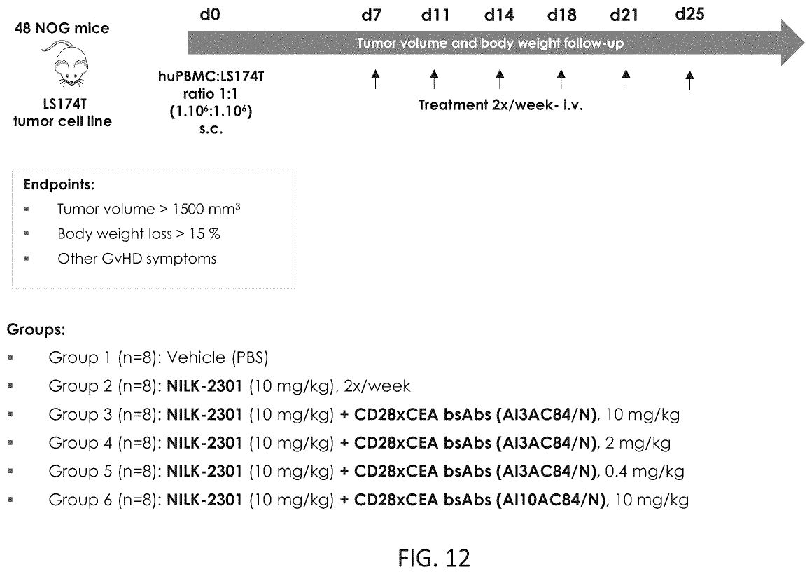

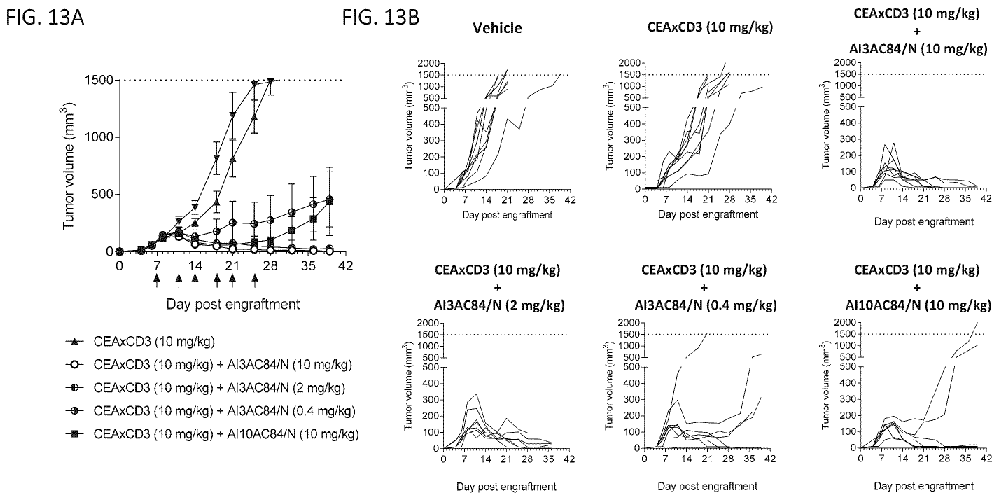

The experimental design is summarized in FIG. 12, where treatment groups and treatment regimens are also shown. Briefly: 1×10⁶ LS174T cells were co-engrafted subcutaneously with 1×10⁶ PBMCs (E:T=1:1) in NOG mice (NOD/Shi-scid/OL-2Rγnull mice, Taconic Biosciences) aged of 7-8 weeks and treatment was started 7 days post engraftment following randomization. CEA×CD3 and CEA×CD28 bsAbs were co-injected at the indicated doses twice weekly for a total of 6 injections. Mice were monitored for tumor development 2 to 3 times a week and tumors were measured by digital caliper until the endpoint of the experiment (tumor volume=1500 mm³ or onset of GvHD symptoms). Tumor volume was calculated using the formula (length×width²)×0.5. FIGS. 13A-13B shows the tumor growth kinetics (mean+SEM) per group (FIG. 13A) and mouse (FIG. 13B).

Only a mild inhibition of tumor growth over the vehicle group was observed in mice treated with CEA×CD3, while all combinations of CD28 bsAb with CEA×CD3 induced important but different anti-tumoral activity. More specifically, the highest dose of AI3AC84/N (10 mg/kg), led to complete tumor regression in 6/8 mice, with palpable but unmeasurable tumors in the remaining 2 mice; the intermediate dose of AI3AC84/N (2 mg/kg) led to strong tumor control and no relapsing tumors, with all tumor volumes eventually falling <50 mm³ (complete tumor eradication was observed only in 1 mouse); the lowest dose (0.4 mg/kg) led to varying tumor control, with half of the mice that eradicated the tumor, and half of the mice for which the treatment only led to delayed tumor growth. The treatment with 10 mg/kg of AI10AC84/N, a CEA×CD28 bsAb that contain an anti-CD28 arm of lower affinity (AI10), was overall less efficacious than the treatment with AI3AC84/N at equivalent dosing, with 2 mice for which only a delayed tumor growth was observed and complete tumor eradication observed only in 3 mice.

### Example 11: In Vitro Characterization of MSLN×CD28 Bispecific Antibodies

Binding of MSLN×CD28 bsAbs to CD28-positive Jurkat cells, MSLN-positive H226 and OVCAR-3 cells or MSLN and CD28 double-negative TIB153 cells.

To demonstrate the binding of MSLN×CD28 κλ-bodies to target cells, a series of experiments based on flow cytometry was performed.

Examples of cells that can be used include MSLN-positive cell lines such as the mesothelioma cell line H226 or the ovarian adenocarcinoma cell line OVCAR-3, CD28-positive cell lines such as the leukemic Jurkat T cells as well as MSLN and CD28 double negative cell lines, such as the leukemic TIB153 cells. Mesothelin cell surface expression for H226 and OVCAR-3 cells was measured at 180′000 and 68′000 MSLN/cell, respectively. Cell staining and binding assessment was performed as described in Example 4.

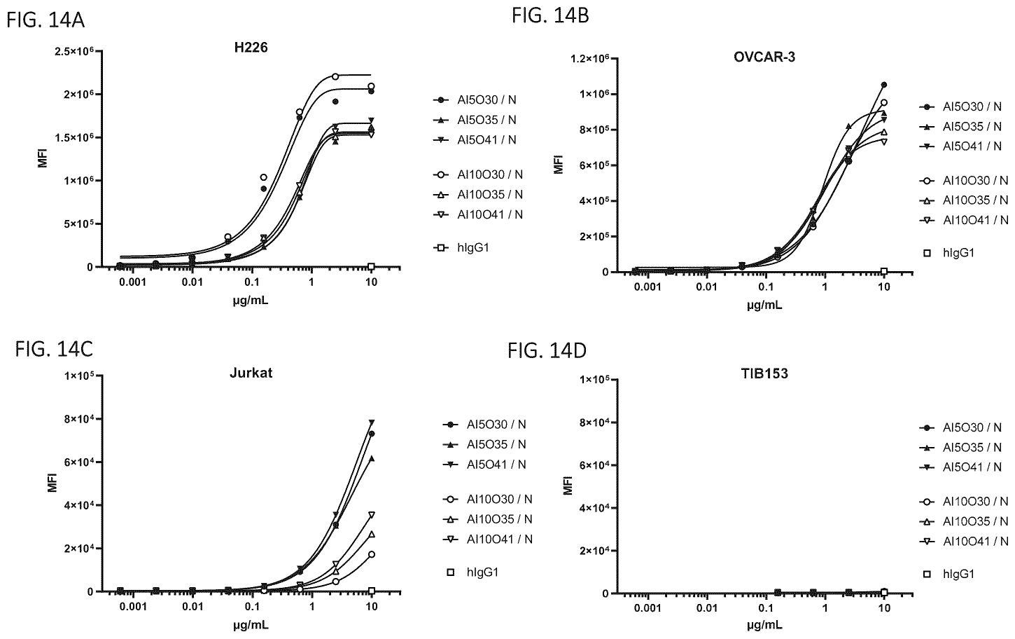

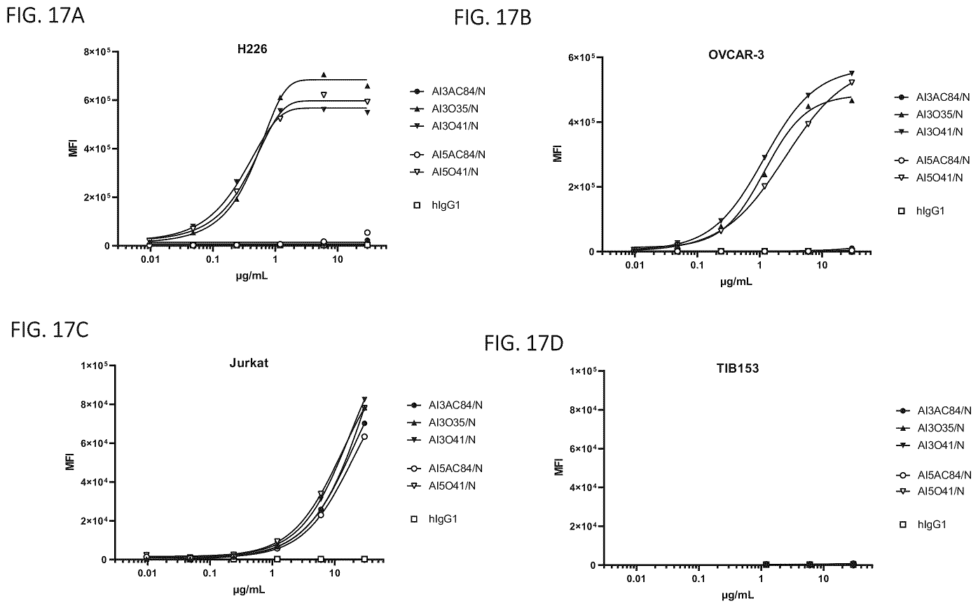

Binding curves of the MSLN×CD28 bsAbs of the invention obtained using H226, OVCAR-3, Jurkat and TIB153 cells are shown in FIGS. 14A, 14B, 14C and 14D, respectively. Binding curves of additional MSLN×CD28 bsAbs of the invention to H226, OVCAR-3, Jurkat and TIB153 cells are shown in FIGS. 17A, 17B, 17C and 17D, respectively. hIgG1 denotes an irrelevant monoclonal antibody of hIgG1 Fc that served as isotype control.

Binding to H226 and OVCAR-3 cells emphasizes how antibodies sharing the same anti-MSLN arm bind similarly and with high affinity to MSLN-positive cells (FIGS. 14A and 14B and FIGS. 17A and 17B). Binding to Jurkat cells reflects the difference in binding affinity of the AI5 vs. AI10 arms (FIG. 14C), but highlights the similarity in binding affinity of the AI5 vs. AI3 arms (FIG. 17C), in line with data generated with CEA×CD28 bsAbs of this invention. The absence of binding signal on TIB-153 suggest that all binding arms of the invention are specific for the designated target (FIGS. 14D and 17D).

### Example 12: T-Cell Dependent Cellular Cytotoxicity (TDCC) Mediated by MSLN×CD28 Bispecific Antibodies

TDCC of MSLN Positive Cell Lines

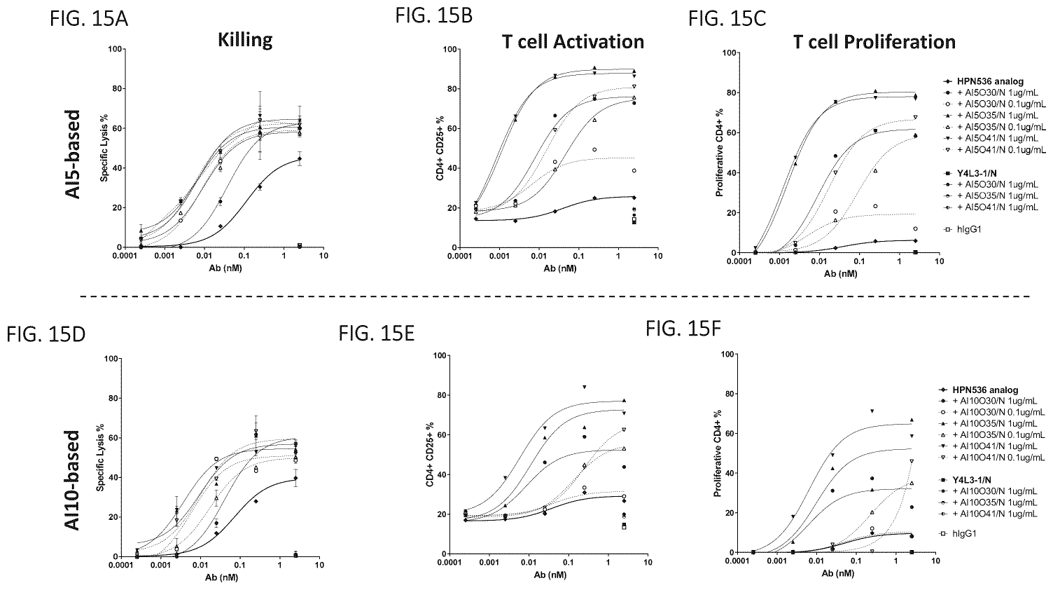

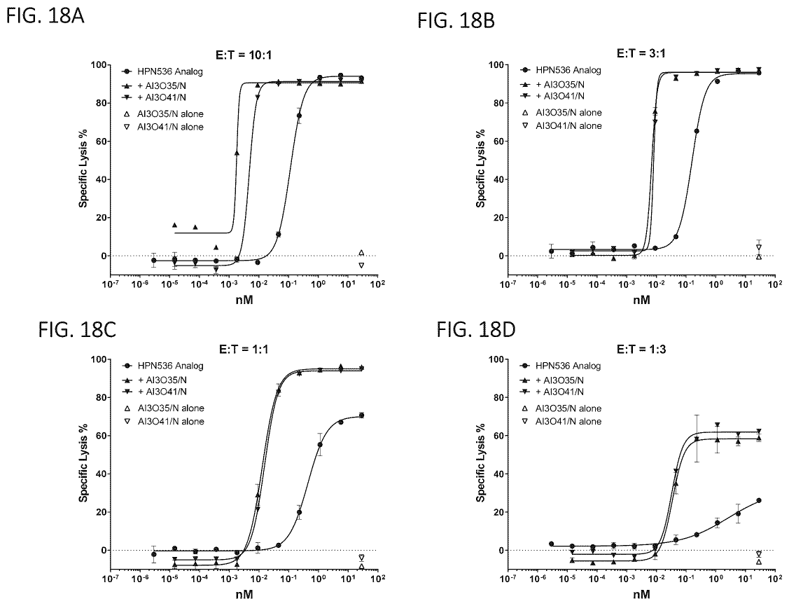

The T-cell dependent cellular cytotoxicity (TDCC) of different MSLN-positive cell lines induced by the MSLN×CD28 bispecific antibodies of the present invention was assessed as described in Example 9. MSLN×CD28 bsAbs were tested in combination with a HPN536 analog, a trispecific MSLN×CD3×HSA molecules capable of redirecting T cells toward MSLN-positive target cells. TDCC curves obtained with H226 cells as target, a dose-range of HPN536 analog and a 1 or 0.1 μg/mL fixed dose of MSLN×CD28 bsAbs are shown on FIGS. 15A and 15D. TDCC curves obtained with OVCAR3 cells as target (at different E:T ratios), a dose-range of HPN536 analog and a 2.5 μg/mL fixed dose of MSLN×CD28 bsAbs are shown on FIGS. 18A-18D.

MSLN×CD28 bsAbs synergized with HPN536 analog to kill MSLN-positive H226 target cells. Synergy can be observed for most of the MSLN×CD28 bsAbs at either of the tested concentration (1 vs. 0.1 μg/mL), but is most striking for bsAbs carrying O35 or O41 as targeting anti-MSLN arms. Nearly the same maximum of specific lysis—between 50 and 60%—of the tumor cells is obtained for AI5O35 and AI5O41 at 0.1 as well as 1 ug/ml, with the lower affinity AI10 arm maximum lysis achieved is between 40 and 60%. Importantly, if HPN536 analog is replaced by Y4L3-1/N, an untargeted CD3 bsAb unable to deliver signal 1 to the effector cells, no killing is induced by the MSLN×CD28 bsAbs, highlighting the importance of primary T cell stimulation (signal 1) for the activity of CD28 bsAbs.

Of note, lowering the E:T ratio (from 10:1, to 3:1, to 1:1 and lastly to 1:3, FIG. 18A-18D) reduced the killing potential of HPN536 analog. If at both 10:1 and 3:1 E:T ratio more than 90% of target cells were killed at the highest concentrations of the HPN536 analog dose range, with E:T ratios equal to 1:1 and 1:3, only 70 and 26% of target cells were respectively killed at the highest MSLN×CD3 concentration. The addition of a MSLN×CD28 bsAb of the invention compensated for such decrease in activity, restoring completely the activity of HPN536 analog at E:T=1:1, and resulting in up to 60% of target cells killed at E:T=1:3.

Up-Regulation of T-Cell Activation Markers Upon Killing of MSLN-Expressing Tumor Cells Induced by the Combination of MSLN×CD3 and MSLN×CD28 bsAbs

The capacity of MSLN×CD28 κλ-bodies to enhance T cell activation in presence of a proper signal 1 was quantified by flow cytometry using antibodies recognizing specific T cell activation markers as described in Example 9. Results of an experiment where H226 cells were co-cultured with PBMC for 48 h at a E:T ratio of 10:1, in presence of a dose range of a MSLN×CD3 (HPN536 analog) and with either 1 μg/mL or 0.1 μg/mL fixed dose of CD28 bsAb are shown in FIGS. 15B and 15E.

Compared to the single treatment with HPN536 analog, the combination of MSLN×CD3 and MSLN×CD28 bsAbs activated CD4+ T cells to a greater extent, with brighter CD25 staining for most of the combination treatments. Differences between the activation level of T cells were observed depending on the dose of CD28 bsAb tested, with 1 μg/mL leading to better T cells activation than 0.1 μg/mL. The importance of both anti-MSLN and anti-CD28 arms is also shown: O35- and O41-based bsAbs better activate the T cells than O30-based bsAb, and the intermediate affinity AI5 arm induced better T cell activation than the low affinity AI10 arm.

Effect of CD28 Costimulation Mediated by MSLN×CD28 bsAbs on T Cell Proliferation in Presence of a MSLN×CD3.

MSLN×CD28 bsAbs were analyzed for their capability to enhance the effects of a MSLN×CD3 in term of induction of T cell proliferation in the presence of MSLN-positive tumor target cells as described in Example 9. The percentage of proliferative CD4+ T cells is shown in FIGS. 15C and 15F. The capacity of a HPN536 analog to induce T cell proliferation was greatly enhanced by the addition of MSLN×CD28 bsAbs, but the greatest effect was obtained with AI5-based bsAbs carrying as targeting arm either O35 or O41, dosed at 1 μg/mL.

Cytokine Released in the Supernatant Upon Enhanced Killing of MSLN-Expressing Tumor Cells by CD28 Costimulation with MSLN×CD28 bsAbs

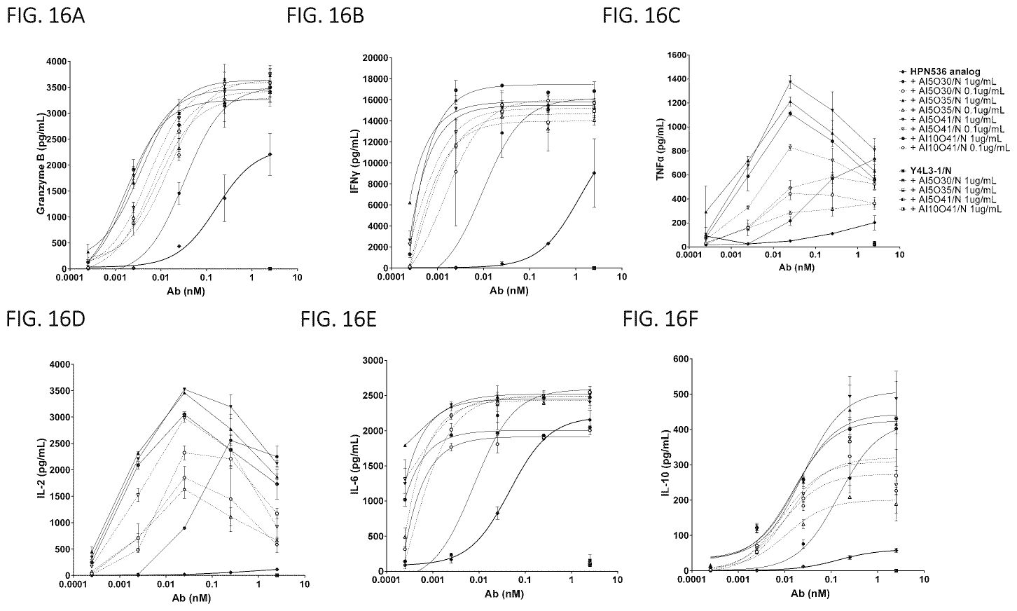

The capacity of the MSLN×CD28 bispecific antibodies of this invention to enhance the release of cytokines by T-cells upon killing of MSLN-expressing tumor cells in presence of a MSLN×CD3 (HPN536 analog) was assessed by quantifying selected cytokines in the supernatant at the end of a TDCC assay, as described in Example 9. Results of an experiment where H226 cells were co-cultured with PBMC at a E:T ratio of 10:1 for 48 h with a dose range of HPN536 analog and either 1 or 0.1 μg/mL fixed dose of MSLN×CD28 are shown in FIGS. 16A-16F.

Single agent treatment with HPN536 analog resulted in moderate cytokine release by T cells, while combination treatment of HPN536 analog with any of the tested MSLN×CD28 bsAbs substantially increased all measured cytokines, especially of IL-2, reflecting the better activation and proliferation of T cells previously observed.

### Example 13: T-Cell Dependent Cellular Cytotoxicity (TDCC) Mediated by TAA×CD3 and TAA×CD28 Bispecific Antibody Combinations Targeting Different Taas

TDCC of CEA MSLN Double Positive Cells

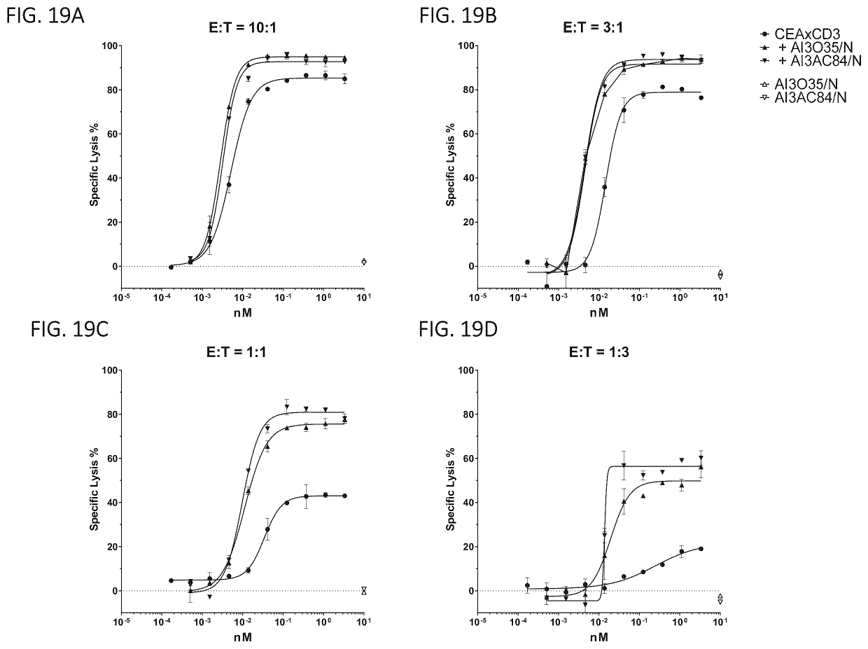

The T-cell dependent cellular cytotoxicity (TDCC) of a CEA/MSLN double positive cell line (HPAC cells, 280′000 CEA/cell and 14′000 CEA/cell) induced by the MSLN×CD28 bispecific antibodies of the present invention was assessed as described in Example 9, using CellTiter-Glo® viability assay as read-out. CD28 bsAbs were tested in combination with a CEA×CD3. TDCC curves obtained with HPAC cells as target (at different E:T ratios), a dose-range of CEA×CD3 and a 2.5 μg/mL fixed dose of either CEA×CD28 or MSLN×CD28 bsAbs are shown on FIGS. 19A-19D.

Both CEA×CD28 and MSLN×CD28 bsAbs synergized equally well with CEA×CD3 to kill CEA/MSLN double-positive HPAC target cells, suggesting that a TAA×CD28 bsAb can be successfully paired to a TAA×CD3 bsAb targeting a different TAA. Synergy can be observed at all tested E:T ratios, but is best highlighted at lower E:T ratios, where the addition of CD28 bsAbs compensated for the lower activity of CEA×CD3.

Importantly, when tested alone (that is, in the absence of signal 1), no killing is induced by the CD28 bsAbs, highlighting the importance of primary T cell stimulation (signal 1) for the activity of CD28 bsAbs.

T-Cell Activation and T Cell Proliferation Induced by the Combination of CD3 and CD28 bsAbs Targeting Different TAAs

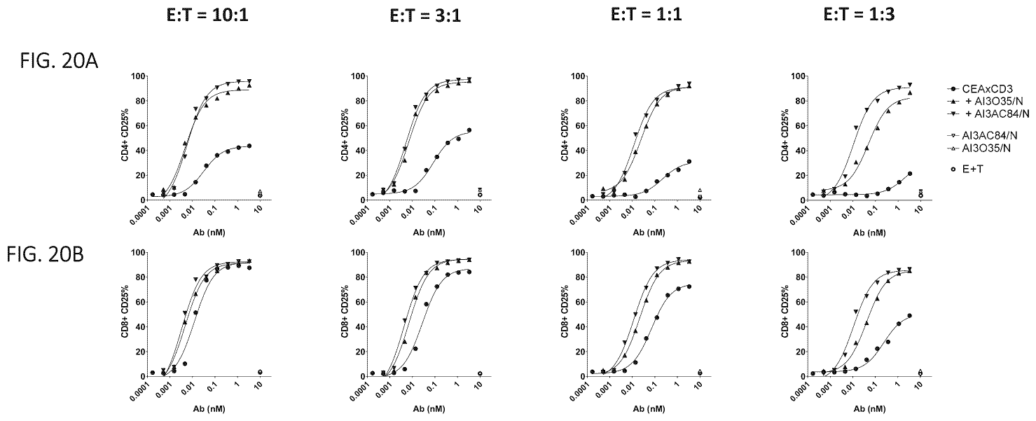

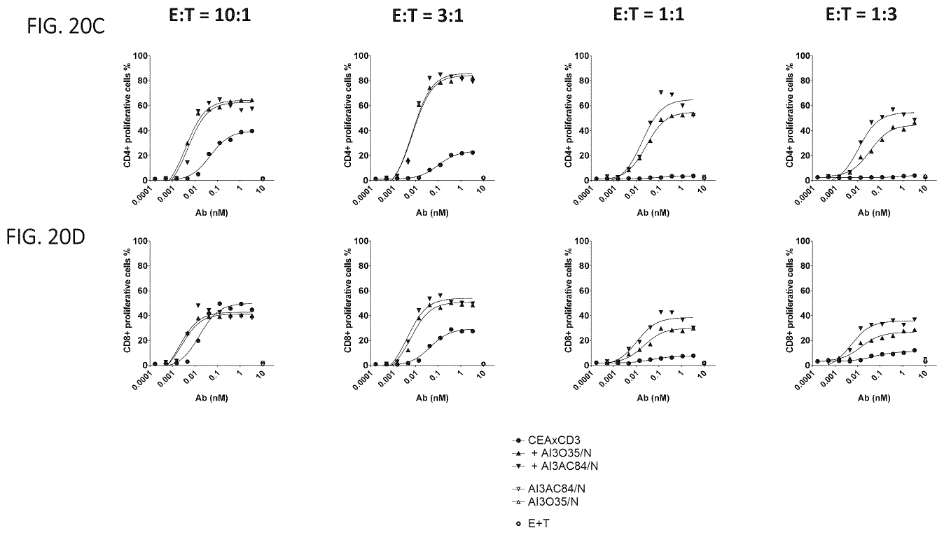

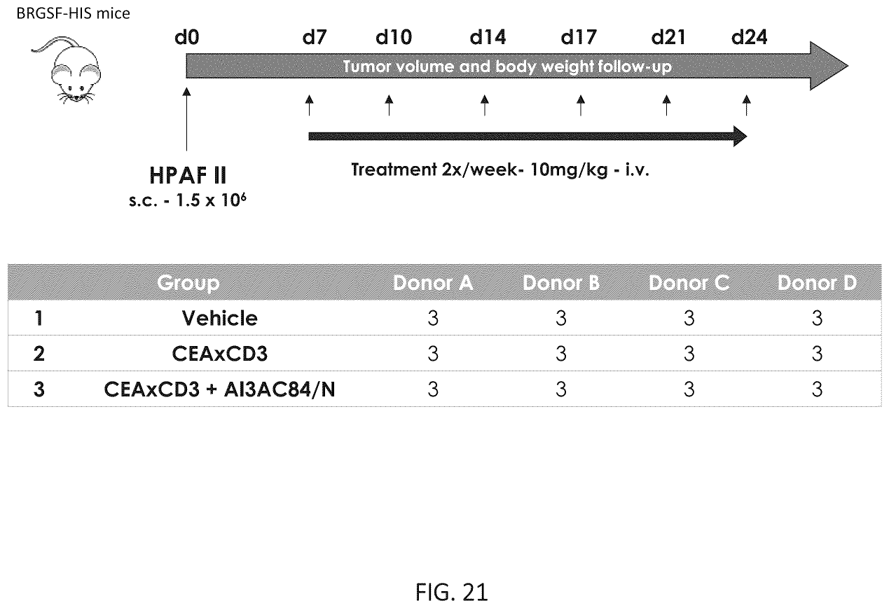

The capacity of CD28 κλ-bodies to enhance T cell activation and proliferation in presence of a proper signal 1 was quantified following the TDCC experiment shown in FIG. 19. T cell activation data is shown in FIGS. 20A and 20B for CD4+ and CD8+ T cells, respectively, while FIGS. 20C and 20D show T cell proliferation of CD4+ and CD8+ T cells, respectively. The different E:T rations tested are shown and are clearly marked at the top of the figure.

Compared to the single treatment with CEA×CD3, the combination of CEA×CD3 with either CEA×CD28 or MSLN×CD28 bsAbs activated CD4+ T cells to a greater extent, with more CD25-positive T cells for both combination treatments and across the different E:T ratios. Synergy between CD3 and CD28 bsAbs for the activation of CD8+ T cells is more evident at lower E:T ratios, as at E:T>1 the CEA×CD3 bsAb well induces CD8+ T cell activation on its own. Similar levels of T cell activation were observed with the two bsAbs, suggesting that TAA×CD28 bsAb can be successfully paired to a TAA×CD3 bsAb targeting a different TAA.

The proliferative potential of T cells reflected the higher T cell activation observed when combinations were used and led to enhanced T cell proliferation induced by the combinations across the 4 E:T ratios tested. Of note, CEA×CD3 alone only minimally induced T cell proliferation at E:T ratios equal or lower than 1, highlighting the benefit of a combination with CD28 bsAbs to induce T cell proliferation even at unfavorable E:T ratio.

### Example 13: Evaluation of CD28 bsAb-Mediated T Cell Costimulation on the Anti-Tumor Activity of CEA×CD3 T-Cell Retargeting bsAb in BRGSF-his Mice

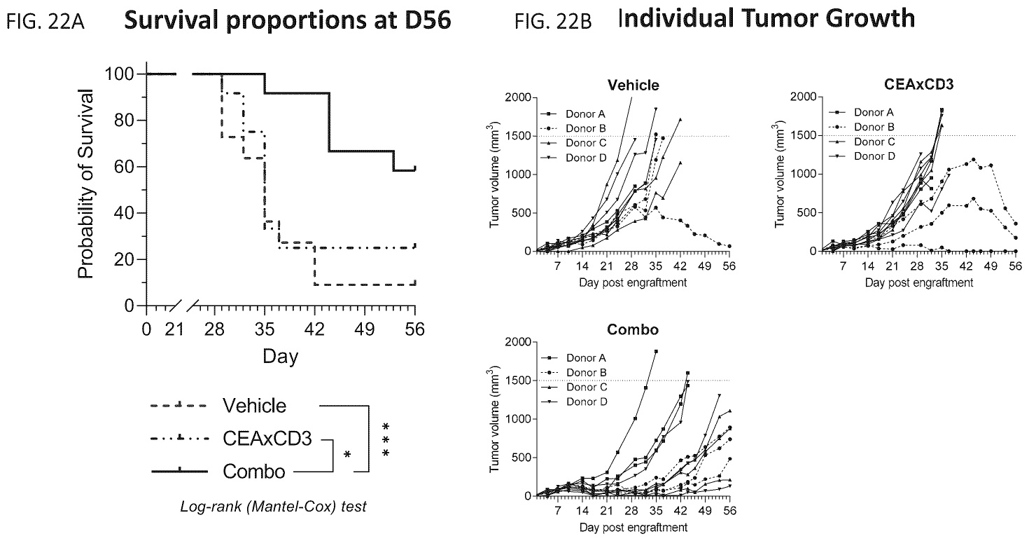

This efficacy study aimed at assessing the anti-tumoral effect of AI3AC84/N when combined with CEA×CD3 in BRGSF-HIS mice subcutaneously engrafted with CEA+HPAF-II tumors. The experimental design is summarized in FIG. 22, where treatment groups and treatment regimens are shown. Briefly: 1.5×10⁶ HPAF-II cells were engrafted subcutaneously in BRGSF-HIS mice (genOway) humanized with 4 distinct CD34+ hematopoietic stem cell donors (9 mice/donor, 3 mice from each donor per treatment group). Treatment was started 7 days post engraftment (average tumor volume ˜85 mm³) following randomization. CEA×CD3 and CEA×CD28 bsAbs were co-injected at 10 mg/kg twice weekly for a total of 6 injections. Mice were monitored for tumor development 2 to 3 times a week and tumors were measured by digital caliper until the endpoint of the experiment (tumor volume=1500 mm³). Tumor volume was calculated using the formula (length×width²)×0.5. FIG. 23A shows the proportion of survival at day 56 post engraftment while FIG. 23B shows the tumor growth kinetics of individual mice in each treatment group.

Only a mild survival advantage was measured for mice treated with CEA×CD3 over the vehicle group. However, the combinations of AI3AC84/N with CEA×CD3 induced lower tumor growth kinetics and higher chance of survival at day 56 (when the experiment was terminated): 8/12 mice of the CEA×CD3+AI3AC84/N group did not reach the tumor endpoint yet, contrary to only 1/12 and 3/12 mice for the vehicle and CEA×CD3 groups, respectively.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

Additional embodiments of the disclosure include the following:
Embodiment 1. A bispecific antibody comprising:
   a. a first antigen binding domain that binds to CD28; wherein the first antigen binding domain comprises:
      i. a first heavy chain variable region having a complementarity determining region 1 (CDRH1) comprising the amino acid sequence of (SEQ ID NO: 1); a complementarity determining region 2 (CDRH2) comprising the amino acid sequence of (SEQ ID NO: 2); and a complementarity determining region 3 (CDRH3) comprising the amino acid sequence of (SEQ ID NO: 3); and
      ii. a first light chain variable region having:
         1. a CDRL1 comprising the amino acid sequence of SEQ ID NO: 23; a CDRL2 comprising the amino acid sequence of SEQ ID NO: 24; and a CDRL3 comprising the amino acid sequence of SEQ ID NO: 25; or
         2. a CDRL1 comprising the amino acid sequence of SEQ ID NO: 26; a CDRL2 comprising the amino acid sequence of SEQ ID NO: 27; and a CDRL3 comprising the amino acid sequence of SEQ ID NO: 28; or
         3. a CDRL1 comprising the amino acid sequence of SEQ ID NO: 29; a CDRL2 comprising the amino acid sequence of SEQ ID NO: 30; and a CDRL3 comprising the amino acid sequence of SEQ ID NO: 31; or
         4. a CDRL1 comprising the amino acid sequence of SEQ ID NO: 32; a CDRL2 comprising the amino acid sequence of SEQ ID NO: 33; and a CDRL3 comprising the amino acid sequence of SEQ ID NO: 34; or
         5. a CDRL1 comprising the amino acid sequence of SEQ ID NO: 35; a CDRL2 comprising the amino acid sequence of SEQ ID NO: 36; and a CDRL3 comprising the amino acid sequence of SEQ ID NO: 37
         6. a CDRL1 comprising the amino acid sequence of SEQ ID NO: 38; a CDRL2 comprising the amino acid sequence of SEQ ID NO: 39; and a CDRL3 comprising the amino acid sequence of SEQ ID NO: 40; or
         7. a CDRL1 comprising the amino acid sequence of SEQ ID NO: 41; a CDRL2 comprising the amino acid sequence of SEQ ID NO: 42; and a CDRL3 comprising the amino acid sequence of SEQ ID NO: 43; or
         8. a CDRL1 comprising the amino acid sequence of SEQ ID NO: 44; a CDRL2 comprising the amino acid sequence of SEQ ID NO: 45; and a CDRL3 comprising the amino acid sequence of SEQ ID NO: 46; or
         9. a CDRL1 comprising the amino acid sequence of SEQ ID NO: 47; a CDRL2 comprising the amino acid sequence of SEQ ID NO: 48; and a CDRL3 comprising the amino acid sequence of SEQ ID NO: 49; or
         10. a CDRL1 comprising the amino acid sequence of SEQ ID NO: 50; a CDRL2 comprising the amino acid sequence of SEQ ID NO: 51; and a CDRL3 comprising the amino acid sequence of SEQ ID NO: 52; or
         11. a CDRL1 comprising the amino acid sequence of SEQ ID NO: 53; a CDRL2 comprising the amino acid sequence of SEQ ID NO: 54; and a CDRL3 comprising the amino acid sequence of SEQ ID NO: 55; or
         12. a CDRL1 comprising the amino acid sequence of SEQ ID NO: 56; a CDRL2 comprising the amino acid sequence of SEQ ID NO: 57; and a CDRL3 comprising the amino acid sequence of SEQ ID NO: 58; or
         13. a CDRL1 comprising the amino acid sequence of SEQ ID NO: 59; a CDRL2 comprising the amino acid sequence of SEQ ID NO: 60; and a CDRL3 comprising the amino acid sequence of SEQ ID NO: 61; or
         14. a CDRL1 comprising the amino acid sequence of SEQ ID NO: 62; a CDRL2 comprising the amino acid sequence of SEQ ID NO: 63; and a CDRL3 comprising the amino acid sequence of SEQ ID NO: 64; or
         15. a CDRL1 comprising the amino acid sequence of SEQ ID NO: 65; a CDRL2 comprising the amino acid sequence of SEQ ID NO: 66; and a CDRL3 comprising the amino acid sequence of SEQ ID NO: 67; or
         16. a CDRL1 comprising the amino acid sequence of SEQ ID NO: 68; a CDRL2 comprising the amino acid sequence of SEQ ID NO: 69; and a CDRL3 comprising the amino acid sequence of SEQ ID NO: 70; or
         17. a CDRL1 comprising the amino acid sequence of SEQ ID NO: 71; a CDRL2 comprising the amino acid sequence of SEQ ID NO: 72; and a CDRL3 comprising the amino acid sequence of SEQ ID NO: 73; or
         18. a CDRL1 comprising the amino acid sequence of SEQ ID NO: 74; a CDRL2 comprising the amino acid sequence of SEQ ID NO: 75; and a CDRL3 comprising the amino acid sequence of SEQ ID NO: 76; or
         19. a CDRL1 comprising the amino acid sequence of SEQ ID NO: 77; a CDRL2 comprising the amino acid sequence of SEQ ID NO: 78; and a CDRL3 comprising the amino acid sequence of SEQ ID NO: 79; and
   b. a second antigen binding domain that bind a tumor associated antigen (TAA), wherein the second antigen binding domain comprises:
      i. a second heavy chain variable region having a CDR1 comprising the amino acid sequence of SEQ ID NO: 1; a CDR2 comprising the amino acid sequence of SEQ ID NO: 2; and a CDR3 comprising the amino acid sequence of SEQ ID NO: 3.
Embodiment 2. The bispecific antibody of claim 1, wherein the TAA is CEA.
Embodiment 3. The bispecific antibody of claim 2, wherein the second antigen binding domain comprises:
   c. second light chain variable region having:
      i. a CDR1 comprising the amino acid sequence of SEQ ID NO: 8; a CDR2 comprising the amino acid sequence of SEQ ID NO: 9; and a CDR3 comprising the amino acid sequence of SEQ ID NO: 10;
      ii. a CDR1 comprising the amino acid sequence of SEQ ID NO: 11; a CDR2 comprising the amino acid sequence of SEQ ID NO: 12; and a CDR3 comprising the amino acid sequence of SEQ ID NO: 13;
      iii. a CDR1 comprising the amino acid sequence of SEQ ID NO: 14; a CDR2 comprising the amino acid sequence of SEQ ID NO: 15 and a CDR3 comprising the amino acid sequence of SEQ ID NO: 16.
Embodiment 4. The bispecific antibody of any one of the preceding claims, wherein the first heavy chain variable region and the second heavy chain variable region comprises the amino acid sequence of SEQ ID NO: 4.
Embodiment 5. The bispecific antibody of any one of the preceding claims, wherein the first heavy chain and the second heavy chain comprises the amino acid sequence of SEQ ID NO: 6 or SEQ ID NO: 7.
Embodiment 6. The bispecific antibody of claim 1, wherein the first light chain variable region of
   d. part ii (1) comprises the amino acid sequence of SEQ ID NO: 80;
   e. part ii (2) comprises the amino acid sequence of SEQ ID NO: 81;
   f. part ii (3) comprises the amino acid sequence of SEQ ID NO: 82;
   g. part ii (4) comprises the amino acid sequence of SEQ ID NO: 83;
   h. part ii (5) comprises the amino acid sequence of SEQ ID NO: 84
   i. part ii (6) comprises the amino acid sequence of SEQ ID NO: 85;
   j. part ii (7) comprises the amino acid sequence of SEQ ID NO: 86;
   k. part ii (8) comprises the amino acid sequence of SEQ ID NO: 87;
   l. part ii (9) comprises the amino acid sequence of SEQ ID NO: 88;
   m. part ii (10) comprises the amino acid sequence of SEQ ID NO: 89;
   n. part ii (11) comprises the amino acid sequence of SEQ ID NO: 90;
   o. part ii (12) comprises the amino acid sequence of SEQ ID NO: 91;
   p. part ii (13) comprises the amino acid sequence of SEQ ID NO: 92;
   q. part ii (14) comprises the amino acid sequence of SEQ ID NO: 93;
   r. part ii (15) comprises the amino acid sequence of SEQ ID NO: 94;
   s. part ii (16) comprises the amino acid sequence of SEQ ID NO: 95;
   t. part ii (17) comprises the amino acid sequence of SEQ ID NO: 96;
   u. part ii (18) comprises the amino acid sequence of SEQ ID NO: 97; and
   v. part ii (19) comprises the amino acid sequence of SEQ ID NO: 98.
Embodiment 7. The bispecific antibody of claim 1, wherein the first light chain of
   a. part ii (1) comprises the amino acid sequence of SEQ ID NO: 99;
   b. part ii (2) comprises the amino acid sequence of SEQ ID NO: 100;
   c. part ii (3) comprises the amino acid sequence of SEQ ID NO: 101;
   d. part ii (4) comprises the amino acid sequence of SEQ ID NO: 102;
   e. part ii (5) comprises the amino acid sequence of SEQ ID NO: 103;
   f. part ii (6) comprises the amino acid sequence of SEQ ID NO: 104;
   g. part ii (7) comprises the amino acid sequence of SEQ ID NO: 105
   h. part ii (8) comprises the amino acid sequence of SEQ ID NO: 106;
   i. part ii (9) comprises the amino acid sequence of SEQ ID NO: 107;
   j. part ii (10) comprises the amino acid sequence of SEQ ID NO: 108;
   k. part ii (11) comprises the amino acid sequence of SEQ ID NO: 109;
   l. part ii (12) comprises the amino acid sequence of SEQ ID NO: 110;
   m. part ii (13) comprises the amino acid sequence of SEQ ID NO: 111;
   n. part ii (14) comprises the amino acid sequence of SEQ ID NO: 112;
   o. part ii (15) comprises the amino acid sequence of SEQ ID NO: 113;
   p. part ii (16) comprises the amino acid sequence of SEQ ID NO: 114;
   q. part ii (17) comprises the amino acid sequence of SEQ ID NO: 115;
   r. part ii (18) comprises the amino acid sequence of SEQ ID NO: 116; and
   s. part ii (19) comprises the amino acid sequence of SEQ ID NO: 117.
Embodiment 8. The bispecific antibody of claim 3, wherein the second light chain variable region of:
   a. part a (i) comprises the amino acid sequence of SEQ ID NO: 17;
   b. part a (ii) comprises the amino acid sequence of SEQ ID NO: 18; and
   c. part a (iii) comprises the amino acid sequence of SEQ ID NO: 19.
Embodiment 9. The bispecific antibody of claim 3, wherein the second light chain of:
   a. part a (i) comprises the amino acid sequence of SEQ ID NO: 20;
   b. part a (ii) comprises the amino acid sequence of SEQ ID NO: 21; and
   c. part a (iii) comprises the amino acid sequence of SEQ ID NO: 22.
Embodiment 10. The bispecific antibody of any one of the preceding claims wherein the first light chain is a kappa and the second light chain is a lambda.
Embodiment 11. The bispecific antibody of any one of the preceding claims wherein the first light chain is a lambda and the second light chain is a kappa.
Embodiment 12. The bispecific antibody of claim 1, wherein the bispecific antibody comprises an Fc domain comprising one or more amino acid substitutions that reduce binding to an activating Fc receptor and/or reduce effector function.
Embodiment 13. The bispecific antibody of claim 12, wherein the amino acid substitution comprises a L234A and L235A substitution.
Embodiment 14. The bispecific antibody of claim 12, wherein the amino acid substitution comprises a P329A, P329G or P329R substitution.
Embodiment 15. The bispecific antibody of any one of the preceding claims, wherein the antibody has an IgG isotype.
Embodiment 16. The bispecific antibody of any one of the preceding claims, wherein the antibodies are human.
Embodiment 17. The bispecific antibody of any one of the preceding claims, wherein the composition enables tumor-specific T cell activation.
Embodiment 18. A composition comprising the bispecific antibody of any one of the preceding claims.
Embodiment 19. The composition of claim 18 further comprising a CD3×CEA bispecific antibody.
Embodiment 20. The composition of claim 19, wherein the CD3×CEA bispecific antibody comprising two heavy chains comprising the amino acid sequence of SEQ ID NO: 118, a first light chain comprising the amino acid sequence of SEQ ID NO: 119, and a second light chain comprising the amino acid sequence of SEQ ID NO: 120.
Embodiment 21. A method of reducing the proliferation of and/or killing a tumor cell comprising contacting the cell with the composition according to any one of claims 18-20.
Embodiment 22. A method of treating a cancer in a subject comprising administering to the subject the composition according to any one of claims 18-20.
Embodiment 23. An antibody comprising an antigen binding domain that binds to CD28; wherein the antigen binding domain comprises:
   i. a heavy chain variable region having a complementarity determining region 1 (CDRH1) comprising the amino acid sequence of (SEQ ID NO: 1); a complementarity determining region 2 (CDRH2) comprising the amino acid sequence of (SEQ ID NO: 2); and a complementarity determining region 3 (CDRH3) comprising the amino acid sequence of (SEQ ID NO: 3); and
   ii. a light chain variable region having:
      1. a CDRL1 comprising the amino acid sequence of SEQ ID NO: 23; a CDRL2 comprising the amino acid sequence of SEQ ID NO: 24; and a CDRL3 comprising the amino acid sequence of SEQ ID NO: 25; or
      2. a CDRL1 comprising the amino acid sequence of SEQ ID NO: 26; a CDRL2 comprising the amino acid sequence of SEQ ID NO: 27; and a CDRL3 comprising the amino acid sequence of SEQ ID NO: 28; or
      3. a CDRL1 comprising the amino acid sequence of SEQ ID NO: 29; a CDRL2 comprising the amino acid sequence of SEQ ID NO: 30; and a CDRL3 comprising the amino acid sequence of SEQ ID NO: 31; or
      4. a CDRL1 comprising the amino acid sequence of SEQ ID NO: 32; a CDRL2 comprising the amino acid sequence of SEQ ID NO: 33; and a CDRL3 comprising the amino acid sequence of SEQ ID NO: 34; or
      5. a CDRL1 comprising the amino acid sequence of SEQ ID NO: 35; a CDRL2 comprising the amino acid sequence of SEQ ID NO: 36; and a CDRL3 comprising the amino acid sequence of SEQ ID NO: 37;
      6. a CDRL1 comprising the amino acid sequence of SEQ ID NO: 38; a CDRL2 comprising the amino acid sequence of SEQ ID NO: 39; and a CDRL3 comprising the amino acid sequence of SEQ ID NO: 40; or
      7. a CDRL1 comprising the amino acid sequence of SEQ ID NO: 41; a CDRL2 comprising the amino acid sequence of SEQ ID NO: 42; and a CDRL3 comprising the amino acid sequence of SEQ ID NO: 43; or
      8. a CDRL1 comprising the amino acid sequence of SEQ ID NO: 44; a CDRL2 comprising the amino acid sequence of SEQ ID NO: 45; and a CDRL3 comprising the amino acid sequence of SEQ ID NO: 46; or
      9. a CDRL1 comprising the amino acid sequence of SEQ ID NO: 47; a CDRL2 comprising the amino acid sequence of SEQ ID NO: 48; and a CDRL3 comprising the amino acid sequence of SEQ ID NO: 49; or
      10. a CDRL1 comprising the amino acid sequence of SEQ ID NO: 50; a CDRL2 comprising the amino acid sequence of SEQ ID NO: 51; and a CDRL3 comprising the amino acid sequence of SEQ ID NO: 52; or
      11. a CDRL1 comprising the amino acid sequence of SEQ ID NO: 53; a CDRL2 comprising the amino acid sequence of SEQ ID NO: 54; and a CDRL3 comprising the amino acid sequence of SEQ ID NO: 55; or
      12. a CDRL1 comprising the amino acid sequence of SEQ ID NO: 56; a CDRL2 comprising the amino acid sequence of SEQ ID NO: 57; and a CDRL3 comprising the amino acid sequence of SEQ ID NO: 58; or
      13. a CDRL1 comprising the amino acid sequence of SEQ ID NO: 59; a CDRL2 comprising the amino acid sequence of SEQ ID NO: 60; and a CDRL3 comprising the amino acid sequence of SEQ ID NO: 61; or
      14. a CDRL1 comprising the amino acid sequence of SEQ ID NO: 62; a CDRL2 comprising the amino acid sequence of SEQ ID NO: 63; and a CDRL3 comprising the amino acid sequence of SEQ ID NO: 64; or
      15. a CDRL1 comprising the amino acid sequence of SEQ ID NO: 65; a CDRL2 comprising the amino acid sequence of SEQ ID NO: 66; and a CDRL3 comprising the amino acid sequence of SEQ ID NO: 67; or
      16. a CDRL1 comprising the amino acid sequence of SEQ ID NO: 68; a CDRL2 comprising the amino acid sequence of SEQ ID NO: 69; and a CDRL3 comprising the amino acid sequence of SEQ ID NO: 70; or
      17. a CDRL1 comprising the amino acid sequence of SEQ ID NO: 71; a CDRL2 comprising the amino acid sequence of SEQ ID NO: 72; and a CDRL3 comprising the amino acid sequence of SEQ ID NO: 73; or
      18. a CDRL1 comprising the amino acid sequence of SEQ ID NO: 74; a CDRL2 comprising the amino acid sequence of SEQ ID NO: 75; and a CDRL3 comprising the amino acid sequence of SEQ ID NO: 76; or
      19. a CDRL1 comprising the amino acid sequence of SEQ ID NO: 77; a CDRL2 comprising the amino acid sequence of SEQ ID NO: 78; and a CDRL3 comprising the amino acid sequence of SEQ ID NO: 79.
Embodiment 24. The antibody of claim 23, wherein the antibody is a F(ab) fragment, a F(ab′)2 fragment, and Fv fragment or a single chain Fv fragment.
Embodiment 25. The antibody of claim 23, wherein the antibody is monospecific.
Embodiment 26. The antibody of claim 23, wherein the antibody is monovalent.

1. A bispecific antibody comprising:
a. a first antigen binding domain that binds to CD28; wherein the first antigen binding domain comprises:
i. a first heavy chain variable region having a complementarity determining region 1 (CDRH1) comprising the amino acid sequence of (SEQ ID NO: 1); a complementarity determining region 2 (CDRH2) comprising the amino acid sequence of (SEQ ID NO: 2); and a complementarity determining region 3 (CDRH3) comprising the amino acid sequence of (SEQ ID NO: 3); and
ii. a first light chain variable region having:
1. a CDRL1 comprising the amino acid sequence of SEQ ID NO: 23;
a CDRL2 comprising the amino acid sequence of SEQ ID NO: 24; and
a CDRL3 comprising the amino acid sequence of SEQ ID NO: 25;
2. a CDRL1 comprising the amino acid sequence of SEQ ID NO: 26;
a CDRL2 comprising the amino acid sequence of SEQ ID NO: 27; and
a CDRL3 comprising the amino acid sequence of SEQ ID NO: 28;
3. a CDRL1 comprising the amino acid sequence of SEQ ID NO: 29;
a CDRL2 comprising the amino acid sequence of SEQ ID NO: 30; and
a CDRL3 comprising the amino acid sequence of SEQ ID NO: 31;
4. a CDRL1 comprising the amino acid sequence of SEQ ID NO: 32;
a CDRL2 comprising the amino acid sequence of SEQ ID NO: 33; and
a CDRL3 comprising the amino acid sequence of SEQ ID NO: 34;
5. a CDRL1 comprising the amino acid sequence of SEQ ID NO: 35;
a CDRL2 comprising the amino acid sequence of SEQ ID NO: 36; and
a CDRL3 comprising the amino acid sequence of SEQ ID NO: 37;
6. a CDRL1 comprising the amino acid sequence of SEQ ID NO: 38;
a CDRL2 comprising the amino acid sequence of SEQ ID NO: 39; and
a CDRL3 comprising the amino acid sequence of SEQ ID NO: 40;
7. a CDRL1 comprising the amino acid sequence of SEQ ID NO: 41;
a CDRL2 comprising the amino acid sequence of SEQ ID NO: 42; and
a CDRL3 comprising the amino acid sequence of SEQ ID NO: 43;
8. a CDRL1 comprising the amino acid sequence of SEQ ID NO: 44;
a CDRL2 comprising the amino acid sequence of SEQ ID NO: 45; and
a CDRL3 comprising the amino acid sequence of SEQ ID NO: 46;
9. a CDRL1 comprising the amino acid sequence of SEQ ID NO: 47;
a CDRL2 comprising the amino acid sequence of SEQ ID NO: 48; and
a CDRL3 comprising the amino acid sequence of SEQ ID NO: 49;
10. a CDRL1 comprising the amino acid sequence of SEQ ID NO: 50;
a CDRL2 comprising the amino acid sequence of SEQ ID NO: 51; and
a CDRL3 comprising the amino acid sequence of SEQ ID NO: 52;
11. a CDRL1 comprising the amino acid sequence of SEQ ID NO: 53;
a CDRL2 comprising the amino acid sequence of SEQ ID NO: 54; and
a CDRL3 comprising the amino acid sequence of SEQ ID NO: 55;
12. a CDRL1 comprising the amino acid sequence of SEQ ID NO: 56;
a CDRL2 comprising the amino acid sequence of SEQ ID NO: 57; and
a CDRL3 comprising the amino acid sequence of SEQ ID NO: 58;
13. a CDRL1 comprising the amino acid sequence of SEQ ID NO: 59;
a CDRL2 comprising the amino acid sequence of SEQ ID NO: 60; and
a CDRL3 comprising the amino acid sequence of SEQ ID NO: 61;
14. a CDRL1 comprising the amino acid sequence of SEQ ID NO: 62;
a CDRL2 comprising the amino acid sequence of SEQ ID NO: 63; and
a CDRL3 comprising the amino acid sequence of SEQ ID NO: 64;
15. a CDRL1 comprising the amino acid sequence of SEQ ID NO: 65;
a CDRL2 comprising the amino acid sequence of SEQ ID NO: 66; and
a CDRL3 comprising the amino acid sequence of SEQ ID NO: 67; or
16. a CDRL1 comprising the amino acid sequence of SEQ ID NO: 68;
a CDRL2 comprising the amino acid sequence of SEQ ID NO: 69; and
a CDRL3 comprising the amino acid sequence of SEQ ID NO: 70;
17. a CDRL1 comprising the amino acid sequence of SEQ ID NO: 71;
a CDRL2 comprising the amino acid sequence of SEQ ID NO: 72; and
a CDRL3 comprising the amino acid sequence of SEQ ID NO: 73;
18. a CDRL1 comprising the amino acid sequence of SEQ ID NO: 74;
a CDRL2 comprising the amino acid sequence of SEQ ID NO: 75; and
a CDRL3 comprising the amino acid sequence of SEQ ID NO: 76; or
19. a CDRL1 comprising the amino acid sequence of SEQ ID NO: 77;
a CDRL2 comprising the amino acid sequence of SEQ ID NO: 78; and
a CDRL3 comprising the amino acid sequence of SEQ ID NO: 79; and
b. a second antigen binding domain that bind a tumor associated antigen (TAA), wherein the second antigen binding domain comprises:
i. a second heavy chain variable region having a CDRH1 comprising the amino acid sequence of SEQ ID NO: 1; a CDRH2 comprising the amino acid sequence of SEQ ID NO: 2; and a CDRH3 comprising the amino acid sequence of SEQ ID NO: 3.

2. The bispecific antibody of claim 1, wherein the first light chain variable region of:
a. part a. ii. 1. comprises the amino acid sequence of SEQ ID NO: 80;
b. part a. ii. 2. comprises the amino acid sequence of SEQ ID NO: 81;
c. part a. ii. 3. comprises the amino acid sequence of SEQ ID NO: 82;
d. part a. ii. 4. comprises the amino acid sequence of SEQ ID NO: 83;
e. part a. ii. 5. comprises the amino acid sequence of SEQ ID NO: 84;
f. part a. ii. 6. comprises the amino acid sequence of SEQ ID NO: 85;
g. part a. ii. 7. comprises the amino acid sequence of SEQ ID NO: 86;
h. part a. ii. 8. comprises the amino acid sequence of SEQ ID NO: 87;
i. part a. ii. 9. comprises the amino acid sequence of SEQ ID NO: 88;
j. part a. ii. 10. comprises the amino acid sequence of SEQ ID NO: 89;
k. part a. ii. 11. comprises the amino acid sequence of SEQ ID NO: 90;
l. part a. ii. 12. comprises the amino acid sequence of SEQ ID NO: 91;
m. part a. ii. 13. comprises the amino acid sequence of SEQ ID NO: 92;
n. part a. ii. 14. comprises the amino acid sequence of SEQ ID NO: 93;
o. part a. ii. 15. comprises the amino acid sequence of SEQ ID NO: 94;
p. part a. ii. 16. comprises the amino acid sequence of SEQ ID NO: 95;
q. part a. ii. 17. comprises the amino acid sequence of SEQ ID NO: 96;
r. part a. ii. 18. comprises the amino acid sequence of SEQ ID NO: 97; or
s. part a. ii. 19. comprises the amino acid sequence of SEQ ID NO: 98.

3. The bispecific antibody of claim 1, wherein the first light chain of:
a. part a. ii. 1. comprises the amino acid sequence of SEQ ID NO: 99;
b. part a. ii. 2. comprises the amino acid sequence of SEQ ID NO: 100;
c. part a. ii. 3. comprises the amino acid sequence of SEQ ID NO: 101;
d. part a. ii. 4. comprises the amino acid sequence of SEQ ID NO: 102;
e. part a. ii. 5. comprises the amino acid sequence of SEQ ID NO: 103;
f. part a. ii. 6. comprises the amino acid sequence of SEQ ID NO: 104;
g. part a. ii. 7. comprises the amino acid sequence of SEQ ID NO: 105;
h. part a. ii. 8. comprises the amino acid sequence of SEQ ID NO: 106;
i. part a. ii. 9. comprises the amino acid sequence of SEQ ID NO: 107;
j. part a. ii. 10. comprises the amino acid sequence of SEQ ID NO: 108;
k. part a. ii. 11. comprises the amino acid sequence of SEQ ID NO: 109;
l. part a. ii. 12. comprises the amino acid sequence of SEQ ID NO: 110;
m. part a. ii. 13. comprises the amino acid sequence of SEQ ID NO: 111;
n. part a. ii. 14. comprises the amino acid sequence of SEQ ID NO: 112;
o. part a. ii. 15. comprises the amino acid sequence of SEQ ID NO: 113;
p. part a. ii. 16. comprises the amino acid sequence of SEQ ID NO: 114;
q. part a. ii. 17. comprises the amino acid sequence of SEQ ID NO: 115;
r. part a. ii. 18. comprises the amino acid sequence of SEQ ID NO: 116; or
s. part a. ii. 19. comprises the amino acid sequence of SEQ ID NO: 117.

4. The bispecific antibody of claim 1, wherein the TAA is CEA.

5. The bispecific antibody of claim 4, wherein the second antigen binding domain comprises:
ii. a second light chain variable region having:
1. a CDR1 comprising the amino acid sequence of SEQ ID NO: 8;
a CDR2 comprising the amino acid sequence of SEQ ID NO: 9; and
a CDR3 comprising the amino acid sequence of SEQ ID NO: 10;
2. a CDR1 comprising the amino acid sequence of SEQ ID NO: 11;
a CDR2 comprising the amino acid sequence of SEQ ID NO: 12; and
a CDR3 comprising the amino acid sequence of SEQ ID NO: 13; or
3. a CDR1 comprising the amino acid sequence of SEQ ID NO: 14;
a CDR2 comprising the amino acid sequence of SEQ ID NO: 15 and
a CDR3 comprising the amino acid sequence of SEQ ID NO: 16.

6. The bispecific antibody claim 4, wherein the second light chain variable region of:
a. part b. ii. 1. comprises the amino acid sequence of SEQ ID NO: 17;
b. part b. ii. 2. comprises the amino acid sequence of SEQ ID NO: 18; or
c. part b. ii. 3. comprises the amino acid sequence of SEQ ID NO: 19.

7. The bispecific antibody of claim 4, wherein the second light chain of:
a. part b. ii. 1. comprises the amino acid sequence of SEQ ID NO: 20;
b. part b. ii. 2. comprises the amino acid sequence of SEQ ID NO: 21; or
c. part b. ii. 3. comprises the amino acid sequence of SEQ ID NO: 22.

8. The bispecific antibody of claim 1, wherein the TAA is mesothelin (MSLN).

9. The bispecific antibody of claim 8, wherein the second antigen binding domain comprises:
ii. a second light chain variable region having:
1. a CDR1 comprising the amino acid sequence of SEQ ID NO: 128;
a CDR2 comprising the amino acid sequence of SEQ ID NO: 129; and
a CDR3 comprising the amino acid sequence of SEQ ID NO: 130;
2. a CDR1 comprising the amino acid sequence of SEQ ID NO: 131;
a CDR2 comprising the amino acid sequence of SEQ ID NO: 132; and
a CDR3 comprising the amino acid sequence of SEQ ID NO: 133;
3. a CDR1 comprising the amino acid sequence of SEQ ID NO: 134;
a CDR2 comprising the amino acid sequence of SEQ ID NO: 135; and
a CDR3 comprising the amino acid sequence of SEQ ID NO: 136;
4. a CDR1 comprising the amino acid sequence of SEQ ID NO: 137;
a CDR2 comprising the amino acid sequence of SEQ ID NO: 138; and
a CDR3 comprising the amino acid sequence of SEQ ID NO: 139; or
5. a CDR1 comprising the amino acid sequence of SEQ ID NO: 140;
a CDR2 comprising the amino acid sequence of SEQ ID NO: 141; and
a CDR3 comprising the amino acid sequence of SEQ ID NO: 142.

10. The bispecific antibody claim 7, wherein the second light chain variable region of:
a. part b. ii. 1. comprises the amino acid sequence of SEQ ID NO: 143;
b. part b. ii. 2. comprises the amino acid sequence of SEQ ID NO: 144;
c. part b. ii. 3. comprises the amino acid sequence of SEQ ID NO: 145;
d. part b. ii. 4. comprises the amino acid sequence of SEQ ID NO: 146; or
e. part b. ii. 5. comprises the amino acid sequence of SEQ ID NO: 147.

11. The bispecific antibody of claim 7, wherein the second light chain of:
a. part b. ii. 1. comprises the amino acid sequence of SEQ ID NO: 148;
b. part b. ii. 2. comprises the amino acid sequence of SEQ ID NO: 149;
c. part b. ii. 3. comprises the amino acid sequence of SEQ ID NO: 150.
d. part b. ii. 4. comprises the amino acid sequence of SEQ ID NO: 151; or
e. part b. ii. 5. comprises the amino acid sequence of SEQ ID NO: 152.

12. The bispecific antibody of claim 1, wherein the first heavy chain variable region and the second heavy chain variable region comprises the amino acid sequence of SEQ ID NO: 4.

13. The bispecific antibody of claim 1, wherein the first heavy chain and the second heavy chain comprises the amino acid sequence of SEQ ID NO: 6 or SEQ ID NO: 7.

14. The bispecific antibody of claim 1, wherein the first light chain is a kappa and the second light chain is a lambda.

15. The bispecific antibody of claim 1, wherein the first light chain is a lambda and the second light chain is a kappa.

16. The bispecific antibody of claim 1, wherein the bispecific antibody comprises an Fc domain comprising one or more amino acid substitutions that reduce binding to an activating Fc receptor and/or reduce effector function.

17. The bispecific antibody of claim 16, wherein the amino acid substitution comprises a L234A and L235A substitution or a P329A, P329G or P329R substitution.

18. (canceled)

19. The bispecific antibody of claim 1, wherein the antibody has an IgG isotype.

20. The bispecific antibody of claim 1, wherein the antibodies are human.

21. The bispecific antibody of claim 1, wherein the composition enables tumor-specific T cell activation.

22. A composition comprising the bispecific antibody of claim 1.

23. A method of reducing the proliferation of and/or killing a tumor cell comprising contacting the cell with the composition of claim 22.

24. A method of treating a cancer in a subject comprising administering to the subject the composition of claim 22.

25. An antibody comprising an antigen binding domain that binds to CD28; wherein the antigen binding domain comprises:
i. a first heavy chain variable region having a complementarity determining region 1 (CDRH1) comprising the amino acid sequence of (SEQ ID NO: 1); a complementarity determining region 2 (CDRH2) comprising the amino acid sequence of (SEQ ID NO: 2); and a complementarity determining region 3 (CDRH3) comprising the amino acid sequence of (SEQ ID NO: 3); and
ii. a first light chain variable region having:
1. a CDRL1 comprising the amino acid sequence of SEQ ID NO: 23;
a CDRL2 comprising the amino acid sequence of SEQ ID NO: 24; and
a CDRL3 comprising the amino acid sequence of SEQ ID NO: 25;
2. a CDRL1 comprising the amino acid sequence of SEQ ID NO: 26;
a CDRL2 comprising the amino acid sequence of SEQ ID NO: 27; and
a CDRL3 comprising the amino acid sequence of SEQ ID NO: 28;
3. a CDRL1 comprising the amino acid sequence of SEQ ID NO: 29;
a CDRL2 comprising the amino acid sequence of SEQ ID NO: 30; and
a CDRL3 comprising the amino acid sequence of SEQ ID NO: 31;
4. a CDRL1 comprising the amino acid sequence of SEQ ID NO: 32;
a CDRL2 comprising the amino acid sequence of SEQ ID NO: 33; and
a CDRL3 comprising the amino acid sequence of SEQ ID NO: 34;
5. a CDRL1 comprising the amino acid sequence of SEQ ID NO: 35;
a CDRL2 comprising the amino acid sequence of SEQ ID NO: 36; and
a CDRL3 comprising the amino acid sequence of SEQ ID NO: 37;
6. a CDRL1 comprising the amino acid sequence of SEQ ID NO: 38;
a CDRL2 comprising the amino acid sequence of SEQ ID NO: 39; and
a CDRL3 comprising the amino acid sequence of SEQ ID NO: 40;
7. a CDRL1 comprising the amino acid sequence of SEQ ID NO: 41;
a CDRL2 comprising the amino acid sequence of SEQ ID NO: 42; and
a CDRL3 comprising the amino acid sequence of SEQ ID NO: 43;
8. a CDRL1 comprising the amino acid sequence of SEQ ID NO: 44;
a CDRL2 comprising the amino acid sequence of SEQ ID NO: 45; and
a CDRL3 comprising the amino acid sequence of SEQ ID NO: 46;
9. a CDRL1 comprising the amino acid sequence of SEQ ID NO: 47;
a CDRL2 comprising the amino acid sequence of SEQ ID NO: 48; and
a CDRL3 comprising the amino acid sequence of SEQ ID NO: 49;
10. a CDRL1 comprising the amino acid sequence of SEQ ID NO: 50;
a CDRL2 comprising the amino acid sequence of SEQ ID NO: 51; and
a CDRL3 comprising the amino acid sequence of SEQ ID NO: 52;
11. a CDRL1 comprising the amino acid sequence of SEQ ID NO: 53;
a CDRL2 comprising the amino acid sequence of SEQ ID NO: 54; and
a CDRL3 comprising the amino acid sequence of SEQ ID NO: 55;
12. a CDRL1 comprising the amino acid sequence of SEQ ID NO: 56;
a CDRL2 comprising the amino acid sequence of SEQ ID NO: 57; and
a CDRL3 comprising the amino acid sequence of SEQ ID NO: 58;
13. a CDRL1 comprising the amino acid sequence of SEQ ID NO: 59;
a CDRL2 comprising the amino acid sequence of SEQ ID NO: 60; and
a CDRL3 comprising the amino acid sequence of SEQ ID NO: 61;
14. a CDRL1 comprising the amino acid sequence of SEQ ID NO: 62;
a CDRL2 comprising the amino acid sequence of SEQ ID NO: 63; and
a CDRL3 comprising the amino acid sequence of SEQ ID NO: 64;
15. a CDRL1 comprising the amino acid sequence of SEQ ID NO: 65;
a CDRL2 comprising the amino acid sequence of SEQ ID NO: 66; and
a CDRL3 comprising the amino acid sequence of SEQ ID NO: 67; or
16. a CDRL1 comprising the amino acid sequence of SEQ ID NO: 68;
a CDRL2 comprising the amino acid sequence of SEQ ID NO: 69; and
a CDRL3 comprising the amino acid sequence of SEQ ID NO: 70;
17. a CDRL1 comprising the amino acid sequence of SEQ ID NO: 71;
a CDRL2 comprising the amino acid sequence of SEQ ID NO: 72; and
a CDRL3 comprising the amino acid sequence of SEQ ID NO: 73;
18. a CDRL1 comprising the amino acid sequence of SEQ ID NO: 74;
a CDRL2 comprising the amino acid sequence of SEQ ID NO: 75; and
a CDRL3 comprising the amino acid sequence of SEQ ID NO: 76; or
19. a CDRL1 comprising the amino acid sequence of SEQ ID NO: 77;
a CDRL2 comprising the amino acid sequence of SEQ ID NO: 78; and
a CDRL3 comprising the amino acid sequence of SEQ ID NO: 79.

26. The antibody of claim 25, wherein the antibody is a F(ab) fragment, a F(ab′)2 fragment, and Fv fragment or a single chain Fv fragment.

27. The antibody of claim 25, wherein the antibody is monospecific or monovalent.

28. (canceled)


SEQUENCE LISTING (152 sequences)

SEQ ID NO: 1 - protein, 8 aa, synthetic construct
GFTFSSYA

SEQ ID NO: 2 - protein, 8 aa, synthetic construct
ISGSGGST

SEQ ID NO: 3 - protein, 9 aa, synthetic construct
AKSYGAFDY

SEQ ID NO: 4 - protein, 116 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSYGAFDYWGQGTLVTVSS

SEQ ID NO: 5 - protein, 445 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSYGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 6 - protein, 445 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSYGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 7 - protein, 445 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSYGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 8 - protein, 8 aa, synthetic construct
SSNIANGI

SEQ ID NO: 9 - skipped

SEQ ID NO: 10 - protein, 10 aa, synthetic construct
GTWDFSYGIV

SEQ ID NO: 11 - protein, 6 aa, synthetic construct
QNIPRT

SEQ ID NO: 12 - skipped

SEQ ID NO: 13 - protein, 11 aa, synthetic construct
QQVTLDPEEMT

SEQ ID NO: 14 - protein, 8 aa, synthetic construct
SSNIGIGL

SEQ ID NO: 15 - skipped

SEQ ID NO: 16 - protein, 10 aa, synthetic construct
GTWDFSYRVD

SEQ ID NO: 17 - protein, 109 aa, synthetic construct
QSVLTQPPSVSAAPGQKVTISCSGSSSNIANGIVSWYQQLPGTAPKLLIYFDNLRPSGIPDRFSGSKSGTSATLGITGLQTGDEADYYCGTWDFSYGIVFGGGTKLTVL

SEQ ID NO: 18 - protein, 109 aa, synthetic construct
DIQMTQSPSSLSASVGDRVTITCQASQNIPRTINWYQQKPGKAPKLLIYDISNLETGVPSRFSGSGSGTDFTFTISSLQPEDIATYYCQQVTLDPEEMTFGQGTKVEIK

SEQ ID NO: 19 - protein, 109 aa, synthetic construct
QSVLTQPPSVSAAPGQKVTISCSGSSSNIGIGLVSWYQQLPGTAPKLLIYNVNTRPSGIPDRFSGSKSGTSATLGITGLQTGDEADYYCGTWDFSYRVDFGGGTKLTVL

SEQ ID NO: 20 - protein, 215 aa, synthetic construct
QSVLTQPPSVSAAPGQKVTISCSGSSSNIANGIVSWYQQLPGTAPKLLIYFDNLRPSGIPDRFSGSKSGTSATLGITGLQTGDEADYYCGTWDFSYGIVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 21 - protein, 216 aa, synthetic construct
DIQMTQSPSSLSASVGDRVTITCQASQNIPRTINWYQQKPGKAPKLLIYDISNLETGVPSRFSGSGSGTDFTFTISSLQPEDIATYYCQQVTLDPEEMTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 215 aa, synthetic construct
QSVLTQPPSVSAAPGQKVTISCSGSSSNIGIGLVSWYQQLPGTAPKLLIYNVNTRPSGIPDRFSGSKSGTSATLGITGLQTGDEADYYCGTWDFSYRVDFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 23 - protein, 12 aa, synthetic construct
QSVLYSSNNKNY

SEQ ID NO: 24 - skipped

SEQ ID NO: 25 - protein, 9 aa, synthetic construct
QQNLRPPET

SEQ ID NO: 26 - protein, 11 aa, synthetic construct
GDLLTFEGKTY

SEQ ID NO: 27 - skipped

SEQ ID NO: 28 - protein, 11 aa, synthetic construct
MQAHSTRFAFT

SEQ ID NO: 29 - protein, 8 aa, synthetic construct
SSNIGSNT

SEQ ID NO: 30 - skipped

SEQ ID NO: 31 - protein, 12 aa, synthetic construct
AAWDLNPAIGYV

SEQ ID NO: 32 - protein, 11 aa, synthetic construct
DDLLRFSGKTY

SEQ ID NO: 33 - skipped

SEQ ID NO: 34 - protein, 11 aa, synthetic construct
MQASRSKVAFT

SEQ ID NO: 35 - protein, 11 aa, synthetic construct
GDLLSFDGKTY

SEQ ID NO: 36 - skipped

SEQ ID NO: 37 - protein, 11 aa, synthetic construct
MQASKHRFGFT

SEQ ID NO: 38 - protein, 11 aa, synthetic construct
GDLLEFAGKTY

SEQ ID NO: 39 - skipped

SEQ ID NO: 40 - protein, 11 aa, synthetic construct
MQAHGSKIGFT

SEQ ID NO: 41 - protein, 6 aa, synthetic construct
QEIHSH

SEQ ID NO: 42 - skipped

SEQ ID NO: 43 - protein, 10 aa, synthetic construct
QQGFRFAFST

SEQ ID NO: 44 - protein, 12 aa, synthetic construct
QSILYSSSNRNY

SEQ ID NO: 45 - skipped

SEQ ID NO: 46 - protein, 9 aa, synthetic construct
QQNFRPPET

SEQ ID NO: 47 - protein, 12 aa, synthetic construct
QSVLYSSNNKNY

SEQ ID NO: 48 - skipped

SEQ ID NO: 49 - protein, 9 aa, synthetic construct
QQNFRPPET

SEQ ID NO: 50 - protein, 7 aa, synthetic construct
QAVFSLD

SEQ ID NO: 51 - skipped

SEQ ID NO: 52 - protein, 11 aa, synthetic construct
QQGPAFKPDVT

SEQ ID NO: 53 - protein, 6 aa, synthetic construct
QSVSSY

SEQ ID NO: 54 - skipped

SEQ ID NO: 55 - protein, 9 aa, synthetic construct
HQRYRPPET

SEQ ID NO: 56 - protein, 6 aa, synthetic construct
QSIGAY

SEQ ID NO: 57 - skipped

SEQ ID NO: 58 - protein, 9 aa, synthetic construct
QQYKDRPLT

SEQ ID NO: 59 - protein, 11 aa, synthetic construct
GNLLTHDGKTY

SEQ ID NO: 60 - skipped

SEQ ID NO: 61 - protein, 11 aa, synthetic construct
MQANALKFGFT

SEQ ID NO: 62 - protein, 11 aa, synthetic construct
SDLLRFEGKTY

SEQ ID NO: 63 - skipped

SEQ ID NO: 64 - protein, 11 aa, synthetic construct
MQAHASALKFT

SEQ ID NO: 65 - protein, 11 aa, synthetic construct
GNLLTHGGKTY

SEQ ID NO: 66 - skipped

SEQ ID NO: 67 - protein, 11 aa, synthetic construct
MQAHADGFGFT

SEQ ID NO: 68 - protein, 11 aa, synthetic construct
GDLLRFDGKTY

SEQ ID NO: 69 - skipped

SEQ ID NO: 70 - protein, 11 aa, synthetic construct
MQASETAVRFT

SEQ ID NO: 71 - protein, 11 aa, synthetic construct
TDLLTFEGKTY

SEQ ID NO: 72 - skipped

SEQ ID NO: 73 - protein, 11 aa, synthetic construct
MQAHSSKFKFT

SEQ ID NO: 74 - protein, 11 aa, synthetic construct
GDLLSTDGKTY

SEQ ID NO: 75 - skipped

SEQ ID NO: 76 - protein, 11 aa, synthetic construct
MQASGSRFYFT

SEQ ID NO: 77 - protein, 12 aa, synthetic construct
QTLLYKYDNKNY

SEQ ID NO: 78 - skipped

SEQ ID NO: 79 - protein, 9 aa, synthetic construct
HQYLYPAET

SEQ ID NO: 80 - protein, 113 aa, synthetic construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQNLRPPETFGQGTKVEIK

SEQ ID NO: 81 - protein, 114 aa, synthetic construct
DIVMTQTPLSLSVTPGQPASISCKSSGDLLTFEGKTYLYWYLQKPGQPPQLLIYEVSNRFSGVPDRFSGSGSGTDFTLKISRVEAEDVGVYYCMQAHSTRFAFTFGQGTKVEIK

SEQ ID NO: 82 - protein, 111 aa, synthetic construct
QSVLTQPPSASGTPGQRVTISCSGSSSNIGSNTVNWYQQLPGTAPKLLIYSENTRPSGVPDRFSGSKSGTSASLAISGLQSEDEADYYCAAWDLNPAIGYVFGGGTKLTVL

SEQ ID NO: 83 - protein, 114 aa, synthetic construct
DIVMTQTPLSLSVTPGQPASISCKSSDDLLRFSGKTYLYWYLQKPGQPPQLLIYEVSNRFSGVPDRFSGSGSGTDFTLKISRVEAEDVGVYYCMQASRSKVAFTFGQGTKVEIK

SEQ ID NO: 84 - protein, 114 aa, synthetic construct
DIVMTQTPLSLSVTPGQPASISCKSSGDLLSFDGKTYLYWYLQKPGQPPQLLIYEVSNRFSGVPDRFSGSGSGTDFTLKISRVEAEDVGVYYCMQASKHRFGFTFGQGTKVEIK

SEQ ID NO: 85 - protein, 114 aa, synthetic construct
DIVMTQTPLSLSVTPGQPASISCKSSGDLLEFAGKTYLYWYLQKPGQPPQLLIYEVSNRFSGVPDRFSGSGSGTDFTLKISRVEAEDVGVYYCMQAHGSKIGFTFGQGTKVEIK

SEQ ID NO: 86 - protein, 108 aa, synthetic construct
DIQMTQSPSTLSASVGDRVTITCRASQEIHSHLSWYQQKPGKAPKLLIYSTSTLESGVPSRFSGSGSGTEFTLTISSLQPDDFATYYCQQGFRFAFSTFGQGTKVEIK

SEQ ID NO: 87 - protein, 113 aa, synthetic construct
DIVMTQSPDSLGVSLGERATINCKSSQSILYSSSNRNYLAWYQQKPGQPPKLLIYWASTRESGVPDRFSGSGSGTDFTLTISSLQAEDVGVYYCQQNFRPPETFGGGTKVEIK

SEQ ID NO: 88 - protein, 113 aa, synthetic construct
DIVMTQSPDSLAVSLGERATINCKSSQSVLYSSNNKNYLAWYQQKPGQPPKLLIYWASTRESGVPDRFSGSGSGTDFTLTISSLQAEDVGVYYCQQNFRPPETFGGGTKVEIK

SEQ ID NO: 89 - protein, 110 aa, synthetic construct
EIVLTQSPGTLSLSPGERATLSCRASQAVFSLDLHWYQQKPGQAPRLLIYSTSKRATGIPDRFSGSGSGTDFTLTISRLEPEDFAVYYCQQGPAFKPDVTFGQGTKVEIK

SEQ ID NO: 90 - protein, 107 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCHQRYRPPETFGQGTKVEIK

SEQ ID NO: 91 - protein, 107 aa, synthetic construct
DIQMTQSPSSLSASVGDRVTITCRASQSIGAYLNWYQQKPGKAPKLLIYHASDLQSGVPSRFSGSGSGTDFTLTISSLQPEDFATYYCQQYKDRPLTFGQGTKVEIK

SEQ ID NO: 92 - protein, 114 aa, synthetic construct
DIVMTQTPLSLSVTPGQPASISCKSSGNLLTHDGKTYLYWYLQKPGQPPQLLIYEVSNRFSGVPDRFSGSGSGTDFTLKISRVEAEDVGVYYCMQANALKFGFTFGQGTKVEIK

SEQ ID NO: 93 - protein, 114 aa, synthetic construct
DIVMTQTPLSLSVTPGQPASISCKSSSDLLRFEGKTYLYWYLQKPGQPPQLLIYEVSNRFSGVPDRFSGSGSGTDFTLKISRVEAEDVGVYYCMQAHASALKFTFGQGTKVEIK

SEQ ID NO: 94 - protein, 114 aa, synthetic construct
DIVMTQTPLSLSVTPGQPASISCKSSGNLLTHGGKTYLYWYLQKPGQPPQLLIYEVSNRFSGVPDRFSGSGSGTDFTLKISRVEAEDVGVYYCMQAHADGFGFTFGQGTKVEIK

SEQ ID NO: 95 - protein, 114 aa, synthetic construct
DIVMTQTPLSLSVTPGQPASISCKSSGDLLRFDGKTYLYWYLQKPGQPPQLLIYEVSNRFSGVPDRFSGSGSGTDFTLKISRVEAEDVGVYYCMQASETAVRFTFGQGTKVEIK

SEQ ID NO: 96 - protein, 114 aa, synthetic construct
DIVMTQTPLSLSVTPGQPASISCKSSTDLLTFEGKTYLYWYLQKPGQPPQLLIYEVSNRFSGVPDRFSGSGSGTDFTLKISRVEAEDVGVYYCMQAHSSKFKFTFGQGTKVEIK

SEQ ID NO: 97 - protein, 114 aa, synthetic construct
DIVMTQTPLSLSVTPGQPASISCKSSGDLLSTDGKTYLYWYLQKPGQPPQLLIYEVSNRFSGVPDRFSGSGSGTDFTLKISRVEAEDVGVYYCMQASGSRFYFTFGQGTKVEIK

SEQ ID NO: 98 - protein, 113 aa, synthetic construct
DIVMTQSPDSLAVSLGERAIINCKSSQTLLYKYDNKNYLAWYQHKPGQPPKLLFYWASTRESGVPDRFSGSGSGTDFTLTINSLQAEDVAVYYCHQYLYPAETFGQGTKLEIK

SEQ ID NO: 99 - protein, 220 aa, synthetic construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQNLRPPETFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 100 - protein, 221 aa, synthetic construct
DIVMTQTPLSLSVTPGQPASISCKSSGDLLTFEGKTYLYWYLQKPGQPPQLLIYEVSNRFSGVPDRFSGSGSGTDFTLKISRVEAEDVGVYYCMQAHSTRFAFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 101 - protein, 217 aa, synthetic construct
QSVLTQPPSASGTPGQRVTISCSGSSSNIGSNTVNWYQQLPGTAPKLLIYSENTRPSGVPDRFSGSKSGTSASLAISGLQSEDEADYYCAAWDLNPAIGYVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 102 - protein, 221 aa, synthetic construct
DIVMTQTPLSLSVTPGQPASISCKSSDDLLRFSGKTYLYWYLQKPGQPPQLLIYEVSNRFSGVPDRFSGSGSGTDFTLKISRVEAEDVGVYYCMQASRSKVAFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 103 - protein, 221 aa, synthetic construct
DIVMTQTPLSLSVTPGQPASISCKSSGDLLSFDGKTYLYWYLQKPGQPPQLLIYEVSNRFSGVPDRFSGSGSGTDFTLKISRVEAEDVGVYYCMQASKHRFGFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 104 - protein, 221 aa, synthetic construct
DIVMTQTPLSLSVTPGQPASISCKSSGDLLEFAGKTYLYWYLQKPGQPPQLLIYEVSNRFSGVPDRFSGSGSGTDFTLKISRVEAEDVGVYYCMQAHGSKIGFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 105 - protein, 215 aa, synthetic construct
DIQMTQSPSTLSASVGDRVTITCRASQEIHSHLSWYQQKPGKAPKLLIYSTSTLESGVPSRFSGSGSGTEFTLTISSLQPDDFATYYCQQGFRFAFSTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 106 - protein, 220 aa, synthetic construct
DIVMTQSPDSLGVSLGERATINCKSSQSILYSSSNRNYLAWYQQKPGQPPKLLIYWASTRESGVPDRFSGSGSGTDFTLTISSLQAEDVGVYYCQQNFRPPETFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 107 - protein, 220 aa, synthetic construct
DIVMTQSPDSLAVSLGERATINCKSSQSVLYSSNNKNYLAWYQQKPGQPPKLLIYWASTRESGVPDRFSGSGSGTDFTLTISSLQAEDVGVYYCQQNFRPPETFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 108 - protein, 217 aa, synthetic construct
EIVLTQSPGTLSLSPGERATLSCRASQAVFSLDLHWYQQKPGQAPRLLIYSTSKRATGIPDRFSGSGSGTDFTLTISRLEPEDFAVYYCQQGPAFKPDVTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 109 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCHQRYRPPETFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 110 - protein, 214 aa, synthetic construct
DIQMTQSPSSLSASVGDRVTITCRASQSIGAYLNWYQQKPGKAPKLLIYHASDLQSGVPSRFSGSGSGTDFTLTISSLQPEDFATYYCQQYKDRPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 111 - protein, 221 aa, synthetic construct
DIVMTQTPLSLSVTPGQPASISCKSSGNLLTHDGKTYLYWYLQKPGQPPQLLIYEVSNRFSGVPDRFSGSGSGTDFTLKISRVEAEDVGVYYCMQANALKFGFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 112 - protein, 221 aa, synthetic construct
DIVMTQTPLSLSVTPGQPASISCKSSSDLLRFEGKTYLYWYLQKPGQPPQLLIYEVSNRFSGVPDRFSGSGSGTDFTLKISRVEAEDVGVYYCMQAHASALKFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 113 - protein, 221 aa, synthetic construct
DIVMTQTPLSLSVTPGQPASISCKSSGNLLTHGGKTYLYWYLQKPGQPPQLLIYEVSNRFSGVPDRFSGSGSGTDFTLKISRVEAEDVGVYYCMQAHADGFGFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 114 - protein, 221 aa, synthetic construct
DIVMTQTPLSLSVTPGQPASISCKSSGDLLRFDGKTYLYWYLQKPGQPPQLLIYEVSNRFSGVPDRFSGSGSGTDFTLKISRVEAEDVGVYYCMQASETAVRFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 115 - protein, 221 aa, synthetic construct
DIVMTQTPLSLSVTPGQPASISCKSSTDLLTFEGKTYLYWYLQKPGQPPQLLIYEVSNRFSGVPDRFSGSGSGTDFTLKISRVEAEDVGVYYCMQAHSSKFKFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 116 - protein, 221 aa, synthetic construct
DIVMTQTPLSLSVTPGQPASISCKSSGDLLSTDGKTYLYWYLQKPGQPPQLLIYEVSNRFSGVPDRFSGSGSGTDFTLKISRVEAEDVGVYYCMQASGSRFYFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 117 - protein, 220 aa, synthetic construct
DIVMTQSPDSLAVSLGERAIINCKSSQTLLYKYDNKNYLAWYQHKPGQPPKLLFYWASTRESGVPDRFSGSGSGTDFTLTINSLQAEDVAVYYCHQYLYPAETFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 118 - protein, 454 aa, synthetic construct
EVQLVESGGGLVQPGGSLKLSCAASGFTFNTYAMNWVRQAPGKGLEWVGRIRSKYNNYATYYADSVKDRFTISRDDSKNTAYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 119 - protein, 216 aa, synthetic construct
EIVLTQSPATLSLSPGERATLSCRASQSVNSNLNWYQQKPGQAPRLLIYHSNNRPHGIPARFSGSGSGTDFTLTISSLEPEDFAVYYCQQFDYFREYNTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 120 - protein, 215 aa, synthetic construct
QTVVTQEPSLTVSPGGTVTLTCRSSTGAVTTSNYANWFQQKPGQAPRGLIGGTNKRAPGTPARFSGSLLGGKAALTLSGAQPEDEAEYYCALWYKQRWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 121 - protein, 214 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPNTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 122 - protein, 440 aa, synthetic construct
QSVLTQPPSVSAAPGQKVTISCSGSSSNIGIGLVSWYQQLPGTAPKLLIYNVNTRPSGIPDRFSGSKSGTSATLGITGLQTGDEADYYCGTWDFSYRVDFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWQSNGQTENNYKTTPPVLDSDGSFFLYSKLTVDKSRWQQGNVFSCSVMHEALHNHYTQKSLSLSPG

SEQ ID NO: 123 - protein, 451 aa, synthetic construct
QVQLQESGPGLVKPSETLSLTCTVSGGSISSYYWSWIRQPPGKGLEWIGYIYYSGITHYNPSLKSRVTISVDTSKIQFSLKLSSVTAADTAVYYCARWGVRRDYYYYGMDVWGQGTTVTVSSASTKGPSVFPLAPSSKSTSGGTAALGCLVEDYFPEPVTVTWNSGALSSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 124 - protein, 215 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 125 - protein, 223 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSYGAFDYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 126 - protein, 447 aa, synthetic construct
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 127 - protein, 214 aa, synthetic construct
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 128 - protein, 8 aa, synthetic construct
SSNIAHGP

SEQ ID NO: 129 - skipped

SEQ ID NO: 130 - protein, 13 aa, synthetic construct
AAYDLTGWFAYAV

SEQ ID NO: 131 - protein, 9 aa, synthetic construct
SGISVKDYR

SEQ ID NO: 132 - protein, 7 aa, synthetic construct
YKSNSDM

SEQ ID NO: 133 - protein, 12 aa, synthetic construct
MIWHHGHGTSLV

SEQ ID NO: 134 - protein, 9 aa, synthetic construct
SDIRVRDYR

SEQ ID NO: 135 - protein, 7 aa, synthetic construct
YKTDSDK

SEQ ID NO: 136 - protein, 12 aa, synthetic construct
MIWHRTTGTSLV

SEQ ID NO: 137 - protein, 9 aa, synthetic construct
SGINVRDYR

SEQ ID NO: 138 - protein, 7 aa, synthetic construct
YKSASDK

SEQ ID NO: 139 - protein, 13 aa, synthetic construct
MIWHHDSEGHAFV

SEQ ID NO: 140 - protein, 6 aa, synthetic construct
KIGHRA

SEQ ID NO: 141 - skipped

SEQ ID NO: 142 - protein, 12 aa, synthetic construct
QVWDWYSEGGVV

SEQ ID NO: 143 - protein, 123 aa, synthetic construct
QSVLTQPPSASGTPGQRVTISCSGSSSNIAHGPVNWYQQLPGTAPKLLIYATNHRPSGVPDRFSGSKSGTTASLTISGLQSEDEADYYCAAYDLTGWFAYAVFGGGTKLTVLGQPKAAPSVTL

SEQ ID NO: 144 - protein, 118 aa, synthetic construct
QPVLTQPASLSASPGASASLTCTLHSGISVKDYRIYWYQQKPGRPPQYLLRYKSNSDMQQGSGVPSRFSGSKDASANAGILLISGLQSEDEADYYCMIWHHGHGTSLVFGGGTKLTVL

SEQ ID NO: 145 - protein, 118 aa, synthetic construct
QPVLTQPVSLSASPGASVSLTCTLRSDIRVRDYRIFWYQQKPGSPPQYLLRYKTDSDKQQGSGVPSRFSGSKDASANAGILLISGLQSEDEADYYCMIWHRTTGTSLVFGGGTKLTVL

SEQ ID NO: 146 - protein, 119 aa, synthetic construct
QPVLTQPASLSASPGASASLTCTLRSGINVRDYRIFWYQQKPGSPPQYLLRYKSASDKQQGSGVPSRFSGSKDASANAGILLISGLQSEDEADYYCMIWHHDSEGHAFVFGGGTKLTVL

SEQ ID NO: 147 - protein, 120 aa, synthetic construct
SYVLTQPPSVSVAPGKTARITCGGNKIGHRAVHWYQQKPGQAPVLVIYYTYERPSGIPERFSGSNSGNTATLTISRVEAGDEADYYCQVWDWYSEGGVVFGGGTKLTVLGQPKAAPSVTL

SEQ ID NO: 148 - protein, 218 aa, synthetic construct
QSVLTQPPSASGTPGQRVTISCSGSSSNIAHGPVNWYQQLPGTAPKLLIYATNHRPSGVPDRFSGSKSGTTASLTISGLQSEDEADYYCAAYDLTGWFAYAVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 149 - protein, 224 aa, synthetic construct
QPVLTQPASLSASPGASASLTCTLHSGISVKDYRIYWYQQKPGRPPQYLLRYKSNSDMQQGSGVPSRFSGSKDASANAGILLISGLQSEDEADYYCMIWHHGHGTSLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 150 - protein, 224 aa, synthetic construct
QPVLTQPVSLSASPGASVSLTCTLRSDIRVRDYRIFWYQQKPGSPPQYLLRYKTDSDKQQGSGVPSRFSGSKDASANAGILLISGLQSEDEADYYCMIWHRTTGTSLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 151 - protein, 225 aa, synthetic construct
QPVLTQPASLSASPGASASLTCTLRSGINVRDYRIFWYQQKPGSPPQYLLRYKSASDKQQGSGVPSRFSGSKDASANAGILLISGLQSEDEADYYCMIWHHDSEGHAFVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 152 - protein, 215 aa, synthetic construct
SYVLTQPPSVSVAPGKTARITCGGNKIGHRAVHWYQQKPGQAPVLVIYYTYERPSGIPERFSGSNSGNTATLTISRVEAGDEADYYCQVWDWYSEGGVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS